(19) United States
(12) Patent Application Publication
(10) Pub. No.: US 2004/0014943 A1
(43) Pub. Date: Jan. 22, 2004

(54) Antibodies for detecting microorganisms

(75) Inventors: Kenji Matsuyama, Sizuoka (JP); Takashi Shirai, Sizuoka (JP); Takashi Etoh, Longmont, CO (US); Monzur Rahman, Columbia, MD (US)

Correspondence Address: YOUNG & THOMPSON, 745 SOUTH 23RD STREET 2ND FLOOR, ARLINGTON, VA, 22202

(73) Assignee: ASAHI KASEI KOGYO KABUSHIKI KAISHA, OSAKA (JP)

(21) Appl. No.: 10/386,050

(22) Filed: Mar. 12, 2003

Related U.S. Application Data
(63) Continuation-in-part of application No. 09/744,910, filed on May 17, 2001.
Application No. 09/744,910 is a 371 of international application No. PCT/JP99/04122, filed on Jul. 30, 1999.
(63) Continuation-in-part of application No. PCT/JP01/00625, filed on Jan. 31, 2001.
(63) Continuation-in-part of application No. PCT/JP01/00626, filed on Jan. 31, 2001.

(30) Foreign Application Priority Data
Jul. 31, 1998 (JP) 10/230204
Jan. 31, 2000 (JP) 2000-62684
Jan. 31, 2000 (JP) 2000-062729

(51) Int. Cl. C07K 1/00; C07K 14/00; C07K 17/00

(52) U.S. Cl. 530/350

(57) ABSTRACT

An antibody for detection of microorganisms, a method of detection of microorganisms, and a reagent kit for detection of microorganisms, which is species specific for every species of microorganisms and with which all serototypes within the same species can be detected, are provided. Antibody to intracellular molecules with the same function in each type of microorganism, particular antibody to ribosomal protein, that is, ribosomal protein L7/Ll2, is made and antibody that reacts specifically with the microorganism in question is selected.

### BACKGROUND OF THE INVENTION

1. Field of the Invention

The present invention pertains to antibodies useful in the detection of various microorganisms, particularly bacteria, and a method of detecting microorganisms, reagent kits that use said antibodies for the detection of microorganisms, and a method for preparing specific antibodies for detecting microorganisms.

Moreover, the present invention is valuable to the drug industry, particularly for diagnostics for microorganism infections with an emphasis on bacteria.

The present invention also relates to antibodies capable of detecting Chlamydia pneumoniae, which is the causative microorganism of common pneumonia, a detection method of the microorganism, a reagent kit for the detection of the microorganism, and a method for preparing the antibody useful to a detection of the microorganism.

The invention is important to medication, specifically to the diagnosis of a typical pneumonia caused by Chlamydia pneumoniae.

The invention can be useful for detecting the species Chlamydia pneumoniae in test samples, such as from throat swabs, tissue samples, body fluids, experimental solutions and cultures.

The present invention also relates to antibody useful to a detection of microorganism that belongs to Mycoplasma pneumoniae, which is the cause microorganism of common pneumonia, a detection method of the microorganism, a reagent kit for the detection of the microorganism, and a method for preparing the antibody useful to a detection of the microorganism.

The invention is important to pharmaceutical industry specifically to the diagnosis of a typical pneumonia caused by Mycoplasma pneumoniae, and has industrial applicability in the art.

The invention can be useful for detecting the species Mycoplasma pneumoniae in test samples, such as from throat swabs, tissue samples, body fluids, experimental solutions and cultures.

2. Description of Related Art

Diagnosis of microorganism infection is confirmed by detection of the causative pathogen from the infected area or by detection of antibodies to the causative pathogen in serum and body fluids. Detection of the causative pathogen is particularly important in the sense that it makes quick treatment of the patient possible.

Detection of the causative pathogen of infections can be generally classified as cultivation and identification methods, whereby the causative pathogen is separated and cultivated and then identified based on its biochemical properties; genetic diagnosis, whereby amplification by PCR, etc., is performed based on specific genes of the causative pathogen and thus the causative pathogen is detected; or immunological methods, whereby the causative pathogen is detected using a specific reaction of antibody with surface antigen marker of the causative pathogen. However, it takes time to obtain results by cultivation and identification methods or genetic diagnosis methods. Therefore, diagnosis by immunological methods is commonly used because the causative pathogen can be detected in a short amount of time with high sensitivity and the patient can be quickly and appropriately treated.

Depending on the bacteria species, a variety of marker antigens and antibodies can be used, alone or in combination to detect the causative pathogen of infections by conventional immunological methods.

For instance, it is known that lipopolysaccharide (LPS), which is a genus-specific antigen of Chlamydia, is present as an antigen determinant (Stephens, R., et al.: J. Immunol., 128:1083-89, 1982, Caldwell, M. D.: Inf. Immun., 44:306-14, 1984), and antibodies to LPS are used as the reagent antibody in a variety of diagnostic kits, particularly for detection of Chlamydia trachomati's.

Moreover, Ellena M. Peterson et al. (Infection and Immunity, 59(11), 4147-4153, 1991) and Byron E. Batteiger et al. (Infection and Immunity, 53(3), 530-533, 1986) have both reported on monoclonal antibody to major outer membrane protein (MOMP) of the genus Chlamydia.

Publication of unexamined application No. 298/1988 discusses an immunodetection method based on the western blotting method that uses a monoclonal antibody to an approximately 43 kilodalton membrane protein antigen of Mycoplasma pneumonia.

Moreover, a method of preparing monoclonal antibody to Haemophilus influenzae and a diagnostic method that uses said antibody are presented in Publication of unexamined application No. 148859/1987 (Patent No. 64065/1994).

Proteins of approximately 20 kilodaltons isolated from the sodium cholate extract of the outer membrane vesicle of Neisseria gonorrhoeae strain BS4 (NCTC 11922) are entered and preparation of a hybridoma using said substance is disclosed in British Patent Application No. 2,172,704. Moreover, EP 419238 Al describes preparation of a monoclonal antibody which can bind to a protein of approximately 14 kilodaltons prepared by using Neisseria gonorrhoeae as an immunogen and a method for the preparation of such an monoclonal antibody. Moreover, a method of detecting the same Neisseria gonorrhoeae using monoclonal antibody to lipopolysaccharide (LPS) is entered in Canadian Patent Application No. 1,220,147.

Chlamydia pneumoniae is the common causative pathogen of pneumonia through out the world. It is small non-motile Gram-negative bacteria that invade selectively human host and cause diseases without any known animal reservoir. The sero-prevalence is 40 to 50% in the 30-to 40-year old age group (Hyman, Roblin et al. 1995). It causes pharyngitis, bronchitis and mild pneumonia.

The microorganism is very small, obligate parasite and grow within the cytoplasm of host cells. Growth of Chlamydia pneumoniae in tissue culture medium is slow (Godzik, O'Brien et al. 1995), and might take at least 3-5 days or more for the identification of the bacterium in the medium (Essig, Zucs et al. 1997). Therefore, Gram staining method and culture method are not pertinent for the diagnostic method rapidly detecting the causative pathogen. As a rapid diagnosis for Chlamydia, the immunological method using antibody is often used.

It is known that lipopolysaccharide (LPS), which is a genus-specific antigen of Chlamydia, is present as an antigen determinant (Verkooyen, Van Lent et al. 1998), and antibodies to LPS are used as the reagent antibody in various diagnostic kits, particularly for detection of Chlamydia trachomatis.

Moreover, Peterson et al.(Peterson, Cheng et al. 1993; Peterson, de la Maza et al. 1998), and Batteiger et al. (Batteiger, Newhall et al. 1986) have both reported on monoclonal antibody to major outer membrane protein (MOMP) of the genus Chlamydia.

These antibodies were found to be useful to distinguish Chlamydia pneumoniae and Chlamydia trachomatis. Later, these antibodies played an important role in unveiling antigenic differences within the species of Chlamydia pneumoniae. Antigens like these might have advantage for serotyping within the species of Chlamydia pneumoniae, but not for routine diagnosis where all strains of the species are needed to be detect.

A common antigen with common function, that most of the structure is evolutionarily protected in the species of the microorganism, but the antigen that could be used for selectively detecting the differences, was not known hitherto.

The present invention relates to useful protein commonly existing in all microorganisms as molecules having same function, and as protein antigen for obtaining antibody. Generally, these molecules do structurally change in a small scale. If these same functional common molecules structurally change in a large scale, it may bring important bad influence to the survival of the microorganisms.

Though there is a few numbers of commercially available monoclonal antibodies for the detection of the pathogen of Chlamydia, those are not enough. Till not long ago it was believed that only TWAR strain causes pneumonia (Thom and Grayston 1991; U.S. Pat. No. 5,008,186). Recently several sero-variety of the pathogen of Chlamydia have been reported. It is found that LPS or MOMP differ from strain to strain and antibodies to only one serotype do not cover all.

Mycoplasma pneumoniae is the common causative pathogen of pneumonia, tracheobronchitis and pharygitis. It is small Gram-negative bacteria that invade selectively human host and cause diseases. It causes approximately 15-20% of all community-acquired pneumonias in general populations and up to 50% of pneumonias in certain confined groups (Ragnar Norrby 1999).

The microorganism is very small in size, fastidious in culture and forms very small colony on enriched media. Growth of Mycoplasma pneumoniae on enriched agar plates are very slow, and might take at least 21 days or more for the identification (Granato, Poe et al. 1980). Several Mycoplasma species of human origin can produce similar biochemical reactions. On the other hand, lacks of cell wall add further difficulties to recognize them under light microscope (Knudson and MacLeod 1979). Therefore, Gram staining and culture methods are not practiced for detecting this pathogen quickly.

DNA hybridization assay probes directed to genomic sequences for detecting Mycoplasma pneumoniae are mentioned by Hyman et al., Buck et al., and Bernet et al. (Hyman, Yogev et al. 1987; Bernet, Garret et al. 1993; Buck, O'Hara et al. 1993). Probes directed to ribosomal RNA (rRNA) sequences of Mycoplasma pneumoniae are mentioned by Tilton et al. (Tilton, Dias et al. 1980), Yogev et al. (Yogev, Halachmi et al. 1988), Gobel et al. (Gobel, Geiser et al. 1987), Zivin and Monahan (EPO 305145, and Gobel and Stanbridge (EPO 250662). Kai et al. (Kai, Kamiya et al. 1987) and Jensen et al. (Jensen, Sondergard-Andersen et al. 1993) describe primers directed to 16S rRNA sequences of Mycoplasma pneumoniae. All mention probes are designed to DNA of Mycoplasma pneumoniae or DNA of Mycoplasma pneumoniae and Mycoplasma genitalium. As these probes are relatively insensitive, the more recently available amplified techniques e.g. PCR have largely captured them. PCR is highly sensitive and can be done within shorter time than hybridization. However, problems of false positive often rise among asymptomatic carriers who are negative to cultured Mycoplasma pneumoniae, or persons who indicate positive after their disease.

Japanese Patent Application Laid-open No. 63(1988)-298 discusses an immuno-detection method based on the western blotting method, that uses a monoclonal antibody to an approximately 43 kilo Dalton membrane protein antigen of Mycoplasma pneumonia. However, there are problems with the said antibody and detection methods based on that antibody in that species specificity to Mycoplasma pneumoniae is insufficient for proper diagnosis due to low specificity. Moreover, the diagnostic method takes at least five hours to complete (Madsen et al. 1988).

However, there are problems with said antibodies and detection methods based on those antibodies in that species specificity to microorganisms is insufficient for proper diagnosis. The antibodies do not detect all serum types from the many surface antigen present within one species.

Marker antigen used by these prior arts is not necessarily standardized so that the same functional molecules (for instance, protein, LPS or surface polysaccharide component with the same function) which are generally present in cells of various microorganisms can be detected for each species of microorganism using as the marker a molecule that changes during the course of evolution of the microorganism, and an immunodiagnostic method based on the concept of detecting the difference in antigenicity between bacteria species using one molecule is not known.

### SUMMARY OF THE INVENTION

The present invention strives to make ideal microorganism detection and immunodiagnosis possible and present antibody to the same molecule in different microorganisms, particularly antibody to the segment that has cells with the same intracellular functional component of all microorganisms that are to be detected and changes during the course of its evolution using a standard marker antigen, and a method of detecting microorganisms, which is species specific and can cover almost all serum types, a reagent kit for detection of microorganisms that use said antibody, and a method for preparing a specific antibody used for detection of microorganism.

### DETAILED DESCRIPTION OF THE INVENTION

The present inventors discovered a protein with the same function preserved in all microorganisms as a useful antigen protein. Usually, it is expected that such a protein is subjected to a structural change only to a small extent. However, surprisingly, it was found that the antigen epitope of this protein has specificity to certain species or genus of microorganisms and that the antibody for this protein not only has potentialities of being used for specifically identifying species or genus of microorganisms, but also is capable of detecting all serotypes of the subject microorganisms.

The inventors focused on intracellular molecules that are present in all microorganism cells and differ between microorganisms in terms of its amino acid structure, particularly ribosomal protein L7/L12, which is a type of ribosomal protein. Ribosomal protein L7/L12 is a protein with a molecular weight of approximately 13 kilodaltons and is known to exist as an essential ribosomal protein in protein synthesis. Progress has been made in understanding the complete amino acid sequence of several microorganisms in particular, including Escherichia coli and Baccillus subtilus, etc., and 50% to 65% homology of the amino acid sequence between the microorganisms is confirmed.

The inventors focused on the fact that even though there are similarities between different microorganisms in terms of said molecule, this molecule also has a structural segment that is unique to each microorganism and discovered that it is possible to detect various microorganisms with species specificity and to detect all serotypes within the same species by using antibody to said protein. As a result of attempting to develop a technology for immunodiagnosis of microorganism species using antibody specific to, for instance, Haemophilus influenzae, Streptococcus pneumoniae, and Neisseria gonorrhoeae, the inventors completed the present invention upon discovering that antibody specific to said protein of each species of microorganisms can be obtained and species-specific detection of different bacteria is possible using said antibody.

The present invention relates to an antibody used for detecting microorganisms, a method of detecting microorganisms using the antibody, a reagent kit for detecting microorganisms using the antibody, and a method for preparing specific antibodies for detecting microorganisms.

1) Antibodies which are antibodies to ribosomal protein of microorganisms and which react specifically with said microorganisms.

2) The antibodies described in 1) above, where the ribosomal protein of the microorganisms is ribosomal protein L7/L12.

3) The antibodies described in 1) or 2) above, where said microorganisms are microorganisms which cause a sexually transmitted disease (STD).

4) The antibodies described in 1) or 2) above, where said microorganisms are microorganisms which cause respiratory tract infection.

5) The antibody described in 4) above, where the causative microorganisms of respiratory tract infection are microorganisms of Haemophilus influenzae.

6) The antibody described in 4) above, where the causative microorganisms of respiratory tract infection are microorganisms of Streptococcus pneumoniae.

7) The antibody described in 3) above, where the causative microorganisms of sexually transmitted diseases (SATD) are microorganisms of Neisseria gonorrhoeae.

8) The antibody described in 7) above, which is the antibody to ribosomal protein L7/L12 of Neisseria gonorrhoeae and which identifies a continuous amino acid sequence moiety from 5 to 30 amino acids including the 115th alanine in the amino acid sequence of Sequence ID No. 22 of the Sequence Table.

9) A method of detecting microorganisms, which is characterized by the fact that antibody to intracellular molecules that have the same function for a variety of microorganisms is used.

10) A method of detecting microorganisms, which is characterized by the fact that any antibody described in 1) to 8) above is used.

11) A reagent kit for detecting microorganisms, which is characterized by the fact that antibody to intracellular molecules that have the same function for a variety of microorganisms is used.

12) A method of detecting microorganisms, which is characterized by the fact that any antibody described in 1) to 8) above is used.

13) A method of preparing any antibody described in 1) to 8) above, characterized by the fact that ribosomal protein L7/L12 of microorganisms obtained by a gene manipulation procedure or by isolation from microorganisms, peptide moiety thereof, or a synthesized peptide corresponding to the peptide moiety is used as an immune source.

The present invention will now be explained in detail.

SEQ ID NO:1 and SEQ ID NO:2 in the Sequence Table are the DNA sequence of the ribosomal protein L7/L12 gene of Hemophilus influenzae and corresponding amino acid sequence. SEQ ID NO:3 and SEQ ID NO:4 are the DNA sequence of the ribosomal protein L7/L12 gene of Helicobacter pylori and the corresponding amino acid sequence. SEQ ID NO:5 and SEQ ID NO:6 show the DNA sequence and the corresponding amino acid sequence of the ribosomal protein L7/L12 gene of Streptococcus pneumoniae. SEQ ID NO:7 and SEQ ID NO: 8 show the DNA sequence and the corresponding amino acid sequence of the ribosomal protein L7/L12 gene of Neisseria gonorrhoeae. SEQ ID NO:9 and SEQ ID NO:10 show the DNA sequence and the corresponding amino acid sequence of the ribosomal protein L7/L12 gene of Neisseria meningitidis. SEQ ID NO:ll and SEQ ID NO:12 in the Sequence Table are the primers DNA for PCR used to acquire the ribosomal protein L7/L12 gene from Hemophilus influenzae. SEQ ID NO:13 and SEQ ID NO: 14 in the Sequence Table are the primer DNA for PCR used to acquire the ribosomal protein L7/L12 gene from Streptococcus pneumoniae. SEQ ID NO:15 and SEQ ID NO: 16 in the Sequence Table are the primer DNA for PCR used to acquire the ribosomal protein L7/L12 gene from Neisseria gonorrhoeae. SEQ ID NO:17 and SEQ ID NO: 18 show the DNA sequence and the corresponding amino acid sequence of the ribosomal protein L7/L12 gene of Hemophilus influenzae. SEQ ID NO:19 and SEQ ID NO: 20 show the DNA sequence and the corresponding amino acid sequence of the ribosomal protein L7/L12 gene of Streptococcus pneumoniae. SEQ ID NO:21 and SEQ ID NO: 22 show the DNA sequence and the corresponding amino acid sequence of the ribosomal protein L7/L12 gene of Neisseria gonorrhoeae.

Furthermore, the left and right terminals of the amino acid sequences entered in the Sequence Table are amino group (referred to below as the N terminal) and carboxyl group terminals (referred to below as the C terminal), respectively, and the left terminal and right terminal of the base sequence is the 5′ terminal and the 3′ terminal, respectively.

Moreover, the series of biomolecular experiments of gene preparation mentioned in this text can be performed by methods entered in standard experimental manuals. “Molecular cloning: A laboratory manual,” Cold Spring Harbor Laboratory Press, Sambrook, J. et al. (1989), is an example of the aforementioned standard experimental manual.

The term microorganism in the present invention refers to all species of microorganisms, including bacteria, yeast, mold, Actinomyces, rickettsia, etc., but bacteria in particular pose a problem in terms of diagnosis of microorganism infections.

The term “antibody which reacts specifically with microorganisms” as used in the present invention means an antibody which reacts specifically with a species or group of microorganisms. An antibody which reacts specifically with a species of microorganisms is particularly useful for the diagnosis of microorganism infection diseases.

In the present invention, causative microorganisms of STD (sexually transmitted disease) include, but are not limited to, Neisseria gonorrhoeae, Chlamydia trachomatis, Candida albicans, Treponema pallidum, and Ureaplasma urealyticum.

In the present invention, causative microorganisms of respiratory tract infection include, but are not limited to, Haemophilus influenzae, Streptococcus pneumoniae, Chlamydia pneumoniae, Mycoplasma pneumoniae, Klebsiella pneumoniae, Staphylococcus aureus, Pseudomonas aeruginosa, Streptococcus sp. GroupA, Mycobacterium tuberculosis, Legionella pneumophila, and Aspergillus spp.

The term antibody in the present invention means a polyclonal antibody or monoclonal antibody that can be made using the entire length or only a partial peptide of said rebosomal protein. Although there are no special restrictions to the peptide length for making the antibody, the segment should be of the length characterizing the ribosomal protein L7/L12 protein, and a peptide of 5 amino acids or longer, particularly 8 amino acids or longer, is preferred. Antiserum containing antibody (polyclonal antibody) that identifies ribosomal protein L7/L12 protein can be obtained by inoculating laboratory animals with adjuvant and a peptide or the full length protein as is or when necessary, after being crosslinked with a carrier protein such as KLH (keyhole-limpet hemocyanin) and BSA (bovine serum albumin) and recovering the serum. Moreover, the antibody can be used after it has been purified from the antiserum. The laboratory animals that are inoculated include sheep, horses, goats, rabbits, mice, rats, etc., and sheep, rabbits, etc., are particularly preferred for preparation of polyclonal antibody. Moreover, monoclonal antibody can also be obtained by conventional methods of making hybridoma cells, but mice are preferred in this case. The entire length of said protein, or its partial peptide consisting amino acid residues of 5 or more, preferably 8 or more, residues that has been fused with GST (glutathione S-transferase), etc., can be purified and used as antigen, or it can be used as antigen without being purified. The antibody can also be the genetic recombination antibody expressed cellularly using immunoglobulin genes that have been separated by a variety of methods in manuals (“Antibodies: A Laboratory manual,” E. Harlow et al., Cold Spring Harbor Laboratory), cloning methods, etc.

Antibody to ribosomal protein L7/L12 protein that can be employed as the marker antigen of the present invention can be obtained by the following 3 methods, and other similar methods as well; the methods, however, are not limited to these.

a) The desired antibody can be acquired by synthesizing a peptide fragment consisting of 5-30 amino acids for microorganisms with a known ribosomal protein L7/L12 genetic sequence and amino acid sequence using the region least similar to the amino acid sequence of said protein of another microorganisms and making polyclonal antibody, or monoclonal antibody, using this peptide fragment as the immune source.

Moreover, it is possible to acquire the entire sequence of said gene by using a conventional genetic procedure, such as gene amplification by PCR with the DNA sequence at both terminals of said known genetic sequence as the probe, or hybridization using the sequence of a homologous segment as the template probe.

Then, a fused gene with another protein gene is constructed and said fused gene is inserted into the host by conventional gene insertion methods using Escherichia coli, etc., as the host and expressed in large quantities. The desired protein antigen can then be acquired by purifying the expressed protein by affinity column methods with antibody to the protein that was used as the fusion protein. In such a case, even if antibody to the amino acid segment retained within the bacteria is acquired, it does not coincide with the purpose of the present invention because the full length of ribosomal protein L7/L12 protein becomes the antigen. Consequently, hybridoma that produces monoclonal antibody to the antigen that has been obtained by this method is acquired by conventional methods and the desired antibody can be obtained by selecting a clone that produces antibody that will react only with the desired microorganisms.

b) First, since there is 50 to 60% homology between bacteria species in terms of their ribosomal protein L7/L12 amino acid sequence, it is possible to easily acquire said protein genes for microorganisms with unknown L9/L12 amino acid sequence by conventional genetic procedures, such as gene amplification of a specific sequence segment by PCR methods based on the sequence of the homologous segments of the amino acid sequence, or hybridization with the homologous segments as the template probe, using bacteria having a known ribosomal protein L7/L12 amino acid sequence.

Then, a fused gene with another protein gene is constructed and said fused gene is inserted into the host by conventional gene insertion methods using Escherichia coli, etc., as the host and expressed in large quantities. The desired protein antigen can then be acquired by purifying the expressed protein by affinity column methods with antibody to the protein that was used as the fusion protein. In such a case, even if antibody to the amino acid segment retained within the bacteria is acquired, it does not coincide with the purpose of the present invention because the full length of ribosomal protein L7/L12 protein becomes the antigen. Consequently, hybridoma that produces monoclonal antibody to the antigen that has been obtained by this method is acquired by conventional methods and the desired antibody can be obtained by selecting a clone that produces antibody that will react only with the desired microorganisms.

c) Ribosomal protein L7/L12 protein that has been purified to a high purity can also be obtained by another method, in the case where the amino acid sequence of the ribosomal protein L7/L12 is unknown whereby a peptide of 5 to 30 amino acids corresponding to the common sequence segment retained in the microorganisms is synthesized from the known amino acid sequence of the ribosomal protein L7/L12, and polyclonal antibody or monoclonal antibody to this peptide sequence is made by conventional methods. Then the highly purified Ribosomal protein L7/L12 from disrupted microorganisms is obtained by affinity column chromatography using said antibody.

If purity of the protein is insufficient, it can be purified by conventional methods, such as ion exchange chromatography, hydrophobic chromatography, gel filtration, etc., after which the eluted fraction of ribosomal protein L7/L12 protein is identified by western blotting, etc., using antibody that was made to obtain the pure fraction. The desired antibody can be obtained by acquiring hybridoma or polyclonal antibody by conventional methods using the pure ribosomal protein L7/Ll2 antigen that has been obtained and selecting hybridoma or polyclonal antibody that will react specifically with the desired bacteria, as in b).

The antibody of the present invention specific to a variety of microorganisms that has been obtained by the methods in a) through c), etc., can be used in a variety of immunoassay methods to obtain diagnostic reagent kits specific to a variety of microorganisms. For example, this antibody can be used in aggregation reactions where antibody is adsorbed on polystyrene latex particles, ELISA, which is a conventional technology performed in a microtiter plate, conventional immunochromatography methods, sandwich assay, whereby said antibody labeled with colored particles or particles that have coloring capability, or with enzyme or phosphor, and magnetic microparticles coated with capture antibody, etc., are used, etc.

The term detection methods for microorganism using antibody means detection methods that use conventional immunoassay such as aggregation reactions where antibody is adsorbed on polystyrene latex particles, ELISA, which is a conventional technology performed in a microtiter plate, conventional immunochromatography methods, sandwich assay, whereby said antibody labeled with colored particles or particles that have coloring capability, or with enzyme or phosphor, and magnetic microparticles coated with capture antibody, etc., are used, etc.

Moreover, the optical immunoassay (OIA) technology described in International Patent Application Japanese Laid-open (Toku-Hyou) No. 509565/1995, in which microorganisms are detected by an optical interference induced by an antibody reaction on the optical thin film which is formed by silicone or silicon nitride, is a useful detection method using an antibody.

Moreover, treatment with an extraction reagent that uses a variety of surfactants, beginning with Triton X-100 and Tween-20, enzyme treatment with an appropriate enzyme, such as protease, etc., methods whereby the known cell structure is crushed, beginning with crushing of the microorganism by physical methods, can be used to extract the intracellular marker antigen from the necessary microorganism in said detection method. However, it is preferred that the extraction conditions be designed using a combination of surfactants, etc., so that the conditions are optimized for extraction of the bacteria in question with a reagent.

Moreover, the term a reagent kit for detection of microorganisms using antibody means a reagent kit that uses said detection method.

For instance, in the case of obtaining the specific antibody to Haemophilus influenzae, which is of extreme diagnostic significance as a causative pathogen of pneumonia, bronchitis, meningitis, etc., the amino acid sequence and DNA sequence of ribosomal protein L7/L12 is entered in data bases, etc.

The amino acid and DNA sequence of ribosomal protein L7/L12 of Haemophilus influenzae are shown in “SEQ ID NO:1 and SEQ ID NO:2.”

Consequently, in the case of this bacteria, it is possible to similarly compare the amino acid sequence of ribosomal protein L7/L12 protein with the same protein of, for instance, Helicobacter pylori, which is shown in “SEQ ID NO:3 and SEQ ID NO:4,” and synthesize a peptide of 5 to 30 amino acids for the segment of low homology and make polyclonal antibody or monoclonal antibody specific to Haemophilus influenzae using this peptide.

In the case of a specific polyclonal antibody, it is preferred that IgG fraction be obtained by purification of the antiserum of immunized laboratory animals with a protein A column, etc., and affinity purification be performed with the synthetic peptide used in immunization of the laboratory animals.

Moreover, PCR primers based on the sequences of N-terminal and C-terminal, for example, the PCR primers shown in SEQ ID NO:11 and SEQ ID NO: 12 in the Sequence Table, were designed from the DNA sequence of ribosomal protein L7/L12 of Haemophilus influenzae. Utilizing homology of the PCR primers, DNA fragments amplified by the PCR method or the like using genomic DNA which is extracted from cultivated Haemophilus influenzae can be acquired according to a conventional method. The entire length of the gene for ribosomal protein L7/L12 protein of Haemophilus influenzae can be acquired by the analysis of the DNA sequence information of these fragments.

The ribosomal protein L7/L12 gene of Haemophilus influenzae thus acquired forms a fusion protein gene with, for instance, GST etc., and expression vector is constructed using the appropriate expression plasmid, Escherichia coli is transformed and large quantities of said protein can be expressed. A suitable amount of the transformed Escherichia coli is cultivated and the crushed bacterial fluid that is recovered is submitted to purification by an affinity column using GST to obtain the ribosomal protein L7/L12 protein and GST fusion protein of Haemophilus influenzae. It is also possible to acquire the target specific monoclonal antibody by establishing a plurality of hybridomas using said protein as is or GST moiety fragments cut from the protein by protease or the like, as an antigen protein and selecting the antibody which exhibits a specific response to Haemophilus influenzae bacteria, a crush fluid of the bacteria, or ribosomal protein L7/Ll2 protein of Haemophilus influenzae.

Moreover, the amino acid sequence and the DNA sequence of ribosomal protein L7/L12 protein of Streptococcus pneumoniae which is also highly significant as a diagnostic agent for respiratory infection diseases as well as Haemophilus influenzae, are known from data bases and the like. The amino acid and DNA sequences of ribosomal protein L7/L12 of Streptococcus pneumoniae are shown in SEQ ID NO:5 and SEQ ID NO:6 of sequence table.

It is therefore possible to acquire a polyclonal antibody or monoclonal antibody which is specific to Streptococcus pneumoniae by designing a PCR primer, the PCR primer shown by Sequence ID No. 13 or 14 in the Sequence Table, for example, based on the sequences of N-terminal and C-terminal of DNA sequence of Ribosomal Protein L7/L12 proteins of Streptococcus pneumoniae in the same manner as in the case of Haemophilus influenzae, and processing thereafter in the same manner as in the case of Haemophilus influenzae.

The hybridoma AMSP-2 which produces the monoclonal antibody specific to Streptococcus pneumoniae has been deposited with the National Institute of Bioscience and Human-Technology, the Agency of Industrial Science and Technology, the Ministry of International Trade and Industry, Japan, on Jul. 28, 1999, with the despostion number FERM BP-6807.

Moreover, although the DNA and amino acid sequences of the ribosomal protein L7/L12 protein of, for instance, Neisseria gonorrhoeae, which is the causative pathogen of gonorrhea and has been shown to have diagnostic significance as a typical causative pathogen of STD, were unknown, a major part of the DNA sequence and amino acid sequence, which was determined by the Neisseria Gonorrhea Genome Project at Oklahoma University of the US, is disclosed on the Internet.

When part of the known DNA sequence of ribosomal protein L7/L12 was used to probe the existence of DNA fragments with a similar sequence, it was found that DNA sequence corresponding to the ribosomal protein L7/L12 gene is present and it was possible to obtain data on its entire DNA sequence. The entire base sequence and corresponding amino acid sequence of the ribosomal protein L7/L12 gene of this Neisseria gonorrhoeae are shown in SEQ ID NO:7 and SEQ ID NO: 8 of the sequence table.

It is therefore possible to acquire the target antibody which is specific to Neisseria gonorrhoeae having the entire or partial Ribosomal Protein L7/L12 protein of Neisseria gonorrhoeae as an antigen by designing a PCR primer, the PCR primer shown by Sequence ID No. 15 or 16 in the Sequence Table, for example, based on the sequences of N-terminal and C-terminal of DNA sequence of Ribosomal Protein L7/L12 protein of Neisseria gonorrhoeae in the same manner as in the case of Haemophilus influenzae, and Streptococcus pneumoniae, and processing thereafter in exactly the same manner as in the case of Haemophilus influenzae or Streptococcus pneumoniae.

Particularly, the gene sequence of ribosomal protein L7/L12 protein of Neisseria meningitidis which belongs to the same Neiseria genus as Neisseria gonorrhoeae is disclosed and readily available on the Internet. The entire base sequence and the corresponding amino acid sequence of the ribosomal protein L7/L12 gene of Neisseria meningitidis are shown in “SEQ ID NO:9 and SEQ ID NO: 10.” Here, comparing the entire base sequence for ribosomal protein L7/L12 genes of Neisseria meningitidis and Neisseria gonorrhoeae, only difference in the amino acid sequence is that Neisseria gonorrhoeae has alanine for the 115th amino acid from the N-terminal, whereas Neisseria meningitidis has glutamic acid. Therefore, it can be concluded that the antibody for the ribosomal protein L7/L12 of Neisseria gonorrhoeae which can specifically detects Neisseria gonorrhoeae is the antibody which identifies alanine at 115 from the N-terminal and the amino acid region including the alanine of ribosomal protein L7/L12 as an epitope.

Antibody made based on the present invention can be used in all known types of immunoassay, such as aggregation whereby said antibody is adsorbed on polystyrene latex, ELISA, which is a conventional technology performed in a microtiter plate, conventional immunochromatography, sandwich assay, whereby said antibody labeled with colored particles or particles that have coloring capability, or enzymes or phosphor, and magnetic particles coated with capture antibody are used, etc.

Moreover, antibody that is made based on the present invention can simultaneously function in any of these immunoassay methods as a so-called-capture antibody that captures said antigen protein in solid or liquid phase and a so-called enzyme-labeled antibody by modification using an enzyme, such as peroxidase and alkali phosphatase, etc., by conventional methods.

### EXAMPLES

The following examples are given to explain the present invention in actual terms, the present invention not being restricted to these examples.

### Example 1

Cloning of Ribosomal Protein L7/L12 Genes from Haemophilus influenzae

After inoculating an appropriate amount of Haemophilus influenzae strain ATCC9334 (IID984) (obtained from Tokyo University School of Medicine Laboratories) in a chocolate agar culture medium, the strain was cultivated for 24 hours in a C02 incubator under conditions of 37° C. and 0.5% CO₂. The colonies that grew were suspended in a TE buffer (manufactured by Wako Pure Chemical Co., Ltd.) to a final concentration of approximately 5×10⁹ CFU/ml. Approximately 1.5 ml of this suspension was transferred to a microcentrifugation tube and centrifuged for 2 minutes at 10,000 rpm. The supernatant was discarded. The sediment was resuspended in 567 μl TE buffer. Then 30 μl 10% sodium dodecylsulfonate (SDS) and 3 μl 20 mg/ml Proteinase K solution were added and thoroughly mixed. The suspension was incubated for another hour at 37° C. Next, after adding 80 μl 10% cetyl trimethyl ammonium bromide/0.7 M NaCl solution and thoroughly mixing the product, it was incubated for 10 minutes at 65° C. Next, 700 μl chloroform-isoamyl alcohol solution at a volume ratio of 24/1 was added and stirred well. The solution was centrifuged for 5 minutes (while being kept at 4° C.) at 12,000 rpm using a microcentrifugation device and the aqueous fraction was transferred to a new microtube. Isopropanol was added to the fraction at 0.6-times its volume and the tube was vigorously shaken to form sediment of the DNA. The white DNA sediment was scooped with a glass rod and transferred to a different microcentrifugation tube containing 1 ml 70% ethanol (cooled to −20° C.)

Next, the product was centrifuged for 5 minutes at 10,000 rpm and the supernatant was gently removed. Then another 1 ml 70% ethanol was added and the product was centrifuged for 5 more minutes.

Once the supernatant had been removed, the sediment was dissolved in 100 μl TE buffer to obtain the DNA solution. The concentration of the genomic DNA solution was determined quantitatively in accordance with E5: Sectrophotometric determination of the amount of DNA or RNA, “Molecular cloning: A laboratory manual,” 1989, Eds. Sambrook, J., Fritsch, E. F., and Maniatis, T., Cold Spring Harbor Laboratory Press.

PCR (polymerase chain reaction) was performed using 10 ng of this genomic DNA. Taq polymerase (Takara Co., ltd., code R001A) was employed for PCR. Then 5 μl of buffer, 4 μl dNTP mixture, and 200 μmol each of synthetic oligonucleotide A shown in SEQ ID NO:ll of the Sequence Table and synthetic oligonucleotide B shown in SEQ ID NO:12 of the Sequence table were added to the enzyme to bring the final volume to 50 μl.

This mixture was cycled 5 times with a TaKaRa PCR Thermal Cycler 480 for 1 minute at 95° C., 2 minutes at 50° C., and 3 minutes at 72° C. and was then cycled 25 times for 1 minute at 95° C., 2 minutes at 60° C., and 3 minutes at 72° C. Electrophoresis was performed in 1.5% agarose gel using some of this PCR product. This product was then stained with ethidium bromide (Nihon Gene Co., ltd.) and observed under ultraviolet rays to confirm amplification of approximately 400 bp cDNA. After digestion treatment with restriction endonucleases BamHI and Xhol, electrophoresis was performed in 1.5% agarose gel and staining with ethidium bromide was carried out. An approximately 370 bp band was cut out from the gel. This band was purified with Suprecol (Takara Co., Ltd.) and then inserted into pGEX-4T-l (Pharmacia), which is a commercial vector. This same vector can function as an expression vector for the desired molecule, which can express fused protein with GST protein, by insertion of the desired gene fragment into the appropriate restriction endonuclease site.

Actually, vector pGEX-4T-l and the previous DNA were mixed together at a molar ratio of 1:3 and DNA was inserted into the vector with T4 DNA ligase (Invitrogen Co.). Vector pGEX-4P-l into which DNA had been inserted was genetically introduced to Escherichia coli one-shot competent cells (Invitrogen Co., Ltd.) and then inoculated in a plate of L-broth (Takara Co., Ltd.) semi-sold culture plate containing 50 μg/ml ampicillin (Sigma). The plate was then set aside at 37° C. for 12 hours and the colonies that grew were selected at random and inoculated into 2 ml L-Broth liquid culture medium containing the same concentration of ampicillin. Shake cultivation was performed at 37° C. for 7 hours and the bacteria was recovered and the plasmid was separated using Wizard Miniprep (Promega) in accordance with the attached literature. The plasmid was cleaved with restriction endonuclease BamHI/XhoI. Insertion of said PCR product was confirmed by cutting out approximately 370 bp DNA. The base sequence of the DNA that had been inserted was determined using said clone.

Determination of the base sequence of the inserted DNA fragment was performed using the Fluorescence Sequencer of Applied Biosystems. The sequence sample was prepared using PRISM, Ready Reaction Dye Terminator Cycle Sequencing Kit (Applied Biosystems). First, 9.5 μl reaction stock solution, 4.0 μl T7 promoter primer at 0.8 μmol/μl (Gibco BRL) and 6.5 μl of template DNA for sequencing at 0.16 μg/μl were added to a microtube with a capacity of 0.5 ml, mixed and superposed with 100 μl mineral oil. PCR amplification was performed for 25 cycles, where one cycle consisted of 30 seconds at 96° C., 15 seconds at 55° C., and 4 minutes at 60° C. The product was then kept at 4° C. for 5 minutes. After the reaction was completed, 80 μl sterilized pure water was added and stirred. The product was centrifuged and the aqueous layer was extracted 3 times with phenol-chloroform. Ten ml 3M sodium acetate (pH 5.2) and 300 μl ethanol were added to 100 μl aqueous layer and stirred. The product was then centrifuged for 15 minutes at room temperature and 14,000 rpm and the sediment was recovered. Once the sediment was washed with 75% ethanol, it was dried under a vacuum for 2 minutes to obtain the sequencing sample. The sequencing sample was dissolved in formamide containing 4 μl 10 mM EDTA and denatured for 2 minutes at 90° C. This was then cooled in ice and submitted to sequencing.

One of the 5 clones obtained had homology of the sequence with the probe used for PCR. In addition, DNA sequences extremely similar to the gene sequence of ribosomal protein L7/L12 gene of the other microorganisms, for example, Neisseria gonorrhoeae, were discovered. The entire base sequence and the corresponding amino acid sequence of the structural gene moiety are as shown in Sequence ID No. 17 and SEQ ID NO: 18 of the Sequence Table. This gene fragment clearly codes for Haemophilus influenzae ribosomal protein L7/L12.

### Example 2

Mass Expression in Escherichia coil and Purification of Ribosomal Protein L7/L12 from Haemophilus influenzae

Fifty milliliters Escherichia coli into which expression vector had been inserted were cultivated overnight in LB at 37° C. Then 500 ml YT medium at concentration that was twice that of the aforementioned culture was heated at 37° C. for 1 hour. Fifty milliliters of the Escherichia coli solution that had been cultivated overnight were introduced to 500 ml of the aforementioned medium. One hour later, 550 μl 100 mM isopropyl □-(D)-thiogalactopyranoside (IPTG) were introduced and cultivated for 4 hours. The product was then recovered and introduced to centrifugation tube at each 250 ml and centrifuged for 10 minutes at 7,000 rpm.

The supernatant was discarded and dissolved in 25 ml each 50 mM TrisHCl at a pH of 7.4 and Lysis buffer containing 25% sucrose.

Furthermore, 1.25 ml 10% Nonidet P-40(NP-40) and 125 μl 1 M MgCl₂ were added and transferred to a plastic tube. Sonication was performed 1 minute×5 times while ice cold. The product was centrifuged for 15 minutes at 12,000 rpm and the supernatant was recovered.

Next, the aforementioned supernatant was adsorbed on a glutathione agarose column conditioned with phosphate-buffered saline (PBS).

Then the column was washed with twice the bed volume using a washing solution containing 20 mM Tris buffer at a pH of 7.4, 4.2 mM MgCl₂, and 1 mM dithiothreithol (DTT). Elution was performed with 50 mM Tris buffer at a pH of 9.6 containing 5 mM glutathione. The protein content in the fraction was determined by the pigment bonding method (Bradford method; Biorad Co.) and the main fraction was acquired.

Purity of the purified ribosomal protein L7/L12/GST fused protein that was obtained was confirmed by electrophoresis to be approximately 75%, showing that a purity satisfactory for an immunogen could be guaranteed.

### Example 3

Preparation of Monoclonal Antibody to Ribosomal Protein L7/L12 of Haemophilus influenzae

First, 100 μg fused protein antigen of ribosomal protein L7/L12/GST of Haemophilus influenzae were dissolved in 200 μl PBS and then 200 μl Freund's complete adjuvant were added and mixed and emulsification was performed. Two-hundred microliters were injected intraperitoneally to immunize mice.

Then the same emulsion antigen was intraperitoneally injected after 2 weeks, after 4 weeks, and after 6 weeks. Two-fold the concentration of antigen emulsion was injected intraperitoneally after 10 weeks and after 14 weeks. The spleen was excised 3 days after the final immunization and submitted to cell fusion.

After thoroughly mixing 2×10⁷ myeloma cells per 10⁸ spleen cells from mice, which had been recovered aseptically, in a glass tube, the mixture was centrifuged for 5 minutes at 1,500 rpm and the supernatant was discarded. The cells were thoroughly mixed.

The myeloma cells used for cell fusion were obtained by cultivation of cell strain NS-1 with an RPMI 1640 culture medium containing 10% FCS, cultivating this product beginning 2 weeks before cell fusion using an RPMI 1640 medium containing 0.13 mM azaguanine, 0.5 μg/ml MC—210, and 10% FCS for 1 weeks, and then further cultivating the cell strain for 1 week with an RPMI 1640 medium containing 10% FCS.

Thirty milliliters of 50 ml of RPMI 640 culture medium that had been kept at 37° C. were added to the mixed cell sample and centrifuged at 1,500 rpm. After removal of the supernatant, 1 ml 50% polyethylene glycol that had been kept at 37° C. was added and stirred for 1 minute. 10 ml RPMI 1640 medium kept at 37° C. were added and the solution was vigorously mixed for approximately 5 minutes as it was suctioned and evacuated from a sterile pipette.

After centrifugation for 5 minutes at 1,000 rpm and removal of the supernatant, 30 ml HAT medium were added to bring the cell concentration to 5×10⁶/ml. This mixture was stirred till uniform and then poured, 0.1 ml at a time, into a 96-well culture plate and cultivated at 37° C. under 7% CO₂. HAT medium was added, 0.1 ml at a time, on Day 1 and at Week 1 and Week 2.

Then the cells that had produced the desired antibody were screened by ELISA.

Solutions of GST fusion ribosomal protein L7/L12 and GST protein of Haemophilus influenzae dissolved in PBS containing 0.05% sodium azide diluted to 10 μg/ml were poured, each 100 Al at a time, into separate 96-well plates and adsorbed overnight at After removal of the supernatant, 200 μl 1% bovine serum albumin solution (in PBS) were added and reacted and blocked for 1 hour at room temperature. After removal of the supernatant, the product was washed with washing solution (0.02% Tween 20, PBS). One-hundred microliters culture solution of fused cells were added to this and reacted for 2 hours at room temperature. The supernatant was removed and washed with washing solution. Next, 100 μl peroxidase-labeled goat anti-mouse IgG antibody solution at a concentration of 50 ng/ml were added and the solution was reacted for 1 hour at room temperature. The supernatant was removed and the product was again washed with washing solution. Then TMB solution (KPL Co., ltd) was added, 100 μl at a time, and the mixture was reacted for 20 minutes at room temperature. After coloration, 100 μl 1 N sulfuric acid were added to stop the reaction and absorbance at 450 nm was determined.

As a result, positive wells that only reacted with GST fusion ribosomal protein L7/L12 protein and did not react with GST protein were detected, and it was concluded that antibody to ribosomal protein L7/L12 protein is present.

Therefore, the cells in the positive wells were recovered and cultivated with HAT medium in a 24-well plastic plate. The fused medium that had been cultivated was diluted with HT medium to a cell count of approximately 20 cells/ml and then mixed with 106 six-week-old mouse thyroid cells suspended in HT Cultivation medium in a 96-well culture plate. Culture was performed for 2 weeks at 37° C. under conditions of 7% C02.

Antibody activity in the culture supernatant was similarly determined by the aforementioned ELISA method and the cells that showed positive reaction with ribosomal protein L7/L12 protein were recovered.

Furthermore, the same dilution test and cloning procedure was repeated to obtain a total of 5 clones of hybridoma HIRB-15.

### Example 4

Reaction of Monoclonal Antibody That Reacts with Ribosomal Protein L7/L12 Protein of Haemophilus influenzae and Neisseria gonorrhoeae and Other Bacteria

Monoclonal antibody was produced and recovered in accordance with standard methods using the positive hybridoma cells obtained as previously described.

Basically, 5×10⁶ cells that had been subcultivated using RPMI 1640 culture medium (containing 10% FCS) were intraperitoneally injected into Balb/C mice that had been intraperitoneally injected with 0.5 ml Pristane 2 weeks earlier. Ascites was recovered 3 weeks later and the centrifugation supernatant was obtained.

The solution containing antibody that was obtained was adsorbed in a Protein A column (5 ml bed, Pharmacia) and rinsed with PBS at 3-times the bed volume. Then elution with citrate buffer at a pH of 3 was performed. The antibody fraction was recovered and the monoclonal antibody that produced by each hybridoma was obtained.

The monoclonal antibody derived from these 5 strains of hybridoma was used in ELISA.

The sandwich assay method was used to assess the monoclonal antibody. The monoclonal antibody that was prepared was used as an enzyme-labeled antibody by being chemically bound to peroxidase.

That is, enzyme labeling was performed in accordance with the method in “Analytical—Biochemistry” 132 (1983), 68-73 with the reagent S-acetylthioacetic acid N-hydroxysuccinimide for binding using horseradish peroxidase (Sigma Grade VI). By means of the ELISA reaction, a solution of commercial anti-Haemophilus influenzae polyclonal antibody which dissolved in PBS containing 0.05% sodium azide, (Biodesign, rabbit) was diluted to a concentration of 10 μg/ml and poured, 100 μl at a time, into a separate 96-well plate and adsorbed overnight at 4° C.

After removal of the supernatant, 200 μl 1% FCS solution (in PBS) were added and reacted and blocked for 1 hour at room temperature. The supernatant was removed and the product was washed with washing solution (0.02% Tween 20, PBS). One-hundred microliters of antigen solution, which had been obtained by adding Triton X-100 to culture solutions of each species of microorganism to a concentration of 0.3% and then extracting the solution for 5 minutes at room temperature, were added to this and reacted for 2 hours at room temperature. The supernatant was removed and the product was further washed with washing solution. Then 100 μl peroxidase-labeled anti-ribosomal protein L7/L12 antibody solution at 5 μg/ml were added and reacted for 1 hour at room temperature. The supernatant was removed and the product was washed with washing solution. TMB (KPL Co.) solution was added, 100 μl at a time, and reacted for 20 minutes at room temperature. After coloration, 100 μl 1 N sulfuric acid were added to stop the reaction. Absorbance at 450 nm was determined.

As a result, as shown in Table 1 it is clear that when monoclonal antibody derived from hybridoma HIRB-2 was used as the enzyme-labeled antibody, all strains of Haemophilus influenzae tested were detected at a sensitivity of 106 bacteria/ml, while reactivity of other bacteria belonging to the genus Neisseria and other microorganisms could not be detected, even at high concentrations of 108 bacteria/ml and therefore, antibody with specific reactivity to Haemophilus influenzae can be obtained by using monoclonal antibody to ribosomal protein L7/L12 protein.

TABLE 1

Results of Detection
(10⁶ cells/ml)

| | | |
|---|---|---|
| Haemophilus influenzae | ATCC9327 | + |
| Haemophilus influenzae | ATCC9334 | + |
| Haemophilus influenzae | ATCC9007 | + |
| Haemophilus influenzae | ATCC9332 | + |
| Haemophilus influenzae | ATCC8142 | + |
| Haemophilus influenzae | ATCC9833 | + |

Results of Detection
(10⁸ cells/ml)

| | | |
|---|---|---|
| Neisseria meningitides | ATCC13090 | − |
| Neisseria lactamica | ATCC30011 | − |
| Neisseria mucosa | ATCC35611 | − |
| Neisseria sicca | ATCC9913 | − |
| Branhamella catarrharis | ATCC25240 | − |
| Neisseria gonorrhoeae | ATCC9793 | − |
| Escherichia coil | ATCC25922 | − |
| Klebsiella pneumoniae | ATCC13883 | − |

(+: Positive; −: Negative)

### Example 5

Cloning of Ribosomal Protein L7/L12 Genes from Streptococcus pneumponiae, Mass Expression in Escherichia coli and Purification of the Same Protein and Preparation of Monoclonal Antibody to the Same Protein

After inoculating an appropriate amount of Streptococcus pneumoniae strain IID555 (obtained from 15 Tokyo University School of Medicine Laboratories) in a blood agar culture medium, the strain was cultivated for 48 hours in an incubator at 37° C. The colonies that grew were suspended in a TE buffer to a final concentration of approximately 5×10⁹ CFU/ml. Approximately 1.5 ml of this suspension was transferred to a microcentrifugation tube and centrifuged for 2 minutes at 10,000 rpm. The supernatant was discarded. The sediment was resuspended in 567 Al TE buffer. Then 30 Al 10% SDS and 3 μl 20 mg/ml Proteinase K solution were added and thoroughly mixed. The suspension was incubated for another hour at 37° C. Next, after adding 80 μl 10% cetyl trimethyl ammonium bromide/0.7 M NaCl solution and thoroughly mixing the product, it was incubated for 10 minutes at 65° C. Next, 700 μl chloroform-isoamyl alcohol solution at a volume ratio of 24/1 was added and stirred well. The solution was centrifuged for 5 minutes (while being kept at 4° C.) at 12,000 rpm using a microcentrifugation device and the aqueous fraction was transferred to a new microtube. Isopropanol was added to the fraction at 0.6-times its volume and the tube was vigorously shaken to form sediment of the DNA. The white DNA sediment was scooped with a glass rod and transferred to a different microcentrifugation tube containing 1 ml 70% ethanol (cooled to −20° C.).

Next, the product was centrifuged for 5 minutes at 10,000 rpm and the supernatant was gently removed. Then another 1 ml 70% ethanol was added and the product was centrifuged for 5 more minutes. Once the supernatant had been removed, the sediment was dissolved in 100 μl TE buffer to obtain the DNA solution. The concentration of the genomic DNA solution was determined quantitatively in accordance with E5: Sectrophotometric determination of the amount of DNA or RNA, “Molecular cloning: A laboratory manual,” 1989, Eds. Sambrook, J., Fritsch, E. F., and Maniatis, T., Cold Spring Harbor Laboratory Press.

PCR was performed using 10 ng of this genomic DNA. Taq polymerase (Takara Co., ltd., code R001A) was employed for PCR. Then 5 μl of buffer attached to enzyme, 4 μl dNTP mixture attached to enzyme, and 200 pmol each of synthetic oligonucleotide C shown in SEQ ID NO:13 of the Sequence Table and synthetic oligonucleotide D shown in SEQ ID NO:14 of the Sequence table were added to the enzyme to bring the final volume to 50 μl.

This mixture was cycled 5 times with a TaKaRa PCR Thermal Cycler 480 for 1 minute at 95° C., 2 minutes at 50° C., and 3 minutes at 72° C. and was then cycled 25 times for 1 minute at 95° C., 2 minutes at 60° C., and 3 minutes at 72° C. Electrophoresis was performed in 1.5% agarose gel using some of this PCR product. This product was then stained with ethidium bromide (Nihon Gene Co., ltd.) and observed under ultraviolet rays to confirm amplification of approximately 400 bp cDNA. After digestion treatment with restriction endonucleases BamHI and Xhol, electrophoresis was performed in 1.5% agarose gel and staining with ethidium bromide was carried out. An approximately 370 bp band was cut out from the gel. This band was purified with Suprec01 (Takara Co., Ltd.) and then inserted into pGEX-6P-l (Pharmacia), which is a commercial vector.

This same vector can function as an expression vector for the desired molecule, which can express fused protein with GST protein, by insertion of the desired gene fragment into the appropriate restriction endonuclease site. Actually, vector pGEX-6P-1 and the previous DNA were mixed together at a molar ratio of 1:5 and DNA was inserted into the vector with T4 DNA ligase (Invitrogen Co.). Vector pGEX-4P-1 into which DNA had been inserted was genetically introduced to Escherichia coli One-Shot Competent Cells (Invitrogen Co., Ltd.) and then inoculated in a plate of L-broth (Takara Co., Ltd.) semi-sold culture plate containing 50 μg/ml ampicillin (Sigma). The plate was then set aside at 37° C. for 12 hours and the colonies that grew were selected at random and inoculated into 2 ml L-Broth liquid culture medium containing the same concentration of ampicillin. Shake cultivation was performed at 37° C. for 8 hours and the bacteria was recovered and the plasmid was separated using Wizard Miniprep in accordance with the attached literature. The plasmid was cleaved with restriction endonuclease BamHI/XhoI. Insertion of said PCR product was confirmed by cutting out approximately 370 bp DNA. The base sequence of the DNA that had been inserted was determined using said clone.

Determination of the base sequence of the inserted DNA fragment was performed using the Fluorescence Sequencer of Applied Biosystems. The sequence sample was prepared using PRISM, Ready Reaction Dye Terminator Cycle Sequencing Kit (Applied Biosystems). First, 9.5 μl reaction stock solution, 4.0 μl T7 promoter primer at 0.8 pmol/pl (Gibco BRL) and 6.5 μl of template DNA for sequencing at 0.16 μg/μl were added to a microtube with a capacity of 0.5 ml, mixed and superposed with 100 Al mineral oil. PCR amplification was performed for 25 cycles, where one cycle consisted of 30 seconds at 96° C., 15 seconds at 55° C., and 4 minutes at 60° C. The product was then kept at 4° C. for 4 minutes. After the reaction was completed, 80 μl sterilized pure water was added and stirred. The product was centrifuged and the aqueous layer was extracted 3 times with phenol-chloroform. Ten microliters 3M sodium acetate (pH 5.2) and 300 μl ethanol were added to 100 μl aqueous layer and stirred. The product was then centrifuged for 15 minutes at room temperature and 14,000 rpm and the sediment was recovered. Once the sediment was washed with 75% ethanol, it was dried under a vacuum for 2 minutes to obtain the sequencing sample. The sequencing sample was dissolved in formamide containing 4 μl 10 mM EDTA and denatured for 2 minutes at 90° C. This was then cooled in ice and submitted to sequencing.

One of the 7 clones obtained had homology of the sequence with the probe used for PCR. In addition, DNA sequences extremely similar to the gene sequence of ribosomal protein L7/L12 gene of the other microorganisms, for example, Neisseria gonorrhoeae, were discovered. The entire base sequence and the corresponding amino acid sequence of the structural gene moiety are as shown in Sequence ID No. 19 and SEQ ID NO: 20 of the Sequence Table. This gene fragment clearly codes for Streptococcus pneumoniae ribosomal protein L7/L12.

50 ml Escherichia coli into which expression vector had been inserted was cultivated overnight in a two-fold concentration YT medium at 37° C. Then, 450 ml of the two-fold concentration YT medium was heated at 37° C. for 1 hour. 50 ml of the Escherichia coli solution that had been cultivated overnight was introduced to 450 ml of the aforementioned medium. After cultivation for one hour at 37° C., 100 μl 500 m mM IPTG was introduced and cultivated for 4 hours at 25° C. The product was then recovered and introduced to a 250 ml centrifugation tube and centrifuged for 20 minutes at 5000 rpm. The supernatant was discarded and dissolved in 25 ml each 50 mM Tris-HCl at a pH of 7.4 and Lysis buffer containing 25% sucrose.

Furthermore, 1.25 ml 10% NP-40 and 125 Al 1 M MgCl₂ were added and transferred to a plastic tube. Sonication was performed 1 minute×5 times while ice cold. The product was centrifuged for 15 minutes at 12,000 rpm and the supernatant was recovered.

Next, the aforementioned supernatant was adsorbed on a glutathione sepharose column (manufactured by Pharmacia) conditioned with PBS. Then, the column was washed with PBS three times the bed volume. Elution was performed with 50 mM Tris-HCl at a pH of 8.0 containing 10 mM glutathione. The protein content in the fraction was determined by the pigment bonding method (Bradford method; Biorad Co.) and the main fraction was acquired. The main fraction was dialyzed three times against 3L PBS. 1 ml of a cleavage buffer containing 500 mM Tris-HCl (pH 7.0), 1.5 M NaCl, 10 mM EDTA, and 10 mM DTT was added to 10 ml of 1 mg/ml solution of the resulting GST fusion ribosomal protein L7/L12. 100 μl of 2 μg/μl PreScission Protease (manufactured by Pharmacia company) was further added and reacted at 4° C. to separate the GST moiety from ribosomal protein L7/L12.

The reaction solution was caused to pass through a glutathione sepharose column which had been conditioned with PBS. The solution coming out from the column was recovered. One-bed volume of PBS was caused to pass through and also recovered. Purity of the purified ribosomal protein L7/L12 protein that was obtained was confirmed by electrophoresis to be approximately 90%, showing that a purity satisfactory for an immunogen could be guaranteed.

First, 100 μg protein antigen of ribosomal protein L7/L12 of Streptococcus pneumoniae was dissolved in 200 μl PBS and then 200 μl Freund's complete adjuvant was added and mixed and emulsification was performed. 200 μl was intraperitoneally injected to immunize mice. Then, the same emulsion antigen was intraperitoneally injected after 2 weeks, after 4 weeks, and after 6 weeks. A two-fold concentration antigen emulsion was further injected intraperitoneally after 10 weeks and after 14 weeks. The spleen was excised 3 days after the final immunization and submitted to cell fusion.

After thoroughly mixing 2×10⁷ myeloma cells per 10⁸ spleen cells from mice, which had been recovered aseptically, in a glass tube, the mixture was centrifuged for 5 minutes at 1500 rpm and the supernatant was discarded. The cells were thoroughly mixed.

The myeloma cells used for cell fusion were obtained by cultivation of cell strain NS-1 with an RPMI 1640 culture medium containing 10% FCS, cultivating this product beginning 2 weeks before cell fusion using an RPMI 1640 medium containing 0.13 mM azaguanine, 0.5 μg/ml MC-210, and 10% FCS for 1 weeks, and then further cultivating the cell strain for 1 week with an RPMI 1640 medium containing 10% FCS. 30 ml of 50 ml RPMI 640 culture medium that had been kept at 37° C. was added to the mixed cell sample and centrifuged at 1,500 rpm. After removal of the supernatant, 1 ml 50% polyethylene glycol that had been kept at 37° C. was added and stirred for 2 minute. 10 ml RPMI 1640 medium kept at 37° C. was added and the solution was vigorously mixed for approximately 5 minutes as it was suctioned and evacuated using a sterile pipette.

After centrifugation for 5 minutes at 1,000 rpm and removal of the supernatant, 30 ml HAT medium were added to bring the cell concentration to 5×10⁶ cells/ml. This mixture was stirred till uniform and then poured, 0.1 ml at a time, into a 96-well culture plate and cultivated at 37° C. under 7% CO₂. HAT medium was added, 0.1 ml at a time, on Day 1 and at Week 1 and Week 2.

Then, the cells that had produced the desired antibody were screened by ELISA. Solutions of ribosomal protein L7/L12 of Streptococcus pneumoniae dissolved in PBS containing 0.05% sodium azide diluted to 10 μg/ml were poured, 100 μl at a time, into separate 96-well plates and adsorbed overnight at 40. After removal of the supernatant, 200 μl 1% FCS solution (in PBS) were added and reacted and blocked for 1 hour at room temperature. The supernatant was removed and the product was washed with a washing solution (0.02% Tween 20, PBS). 100 μl of a culture solution of fusion cells was added and reacted for two hours at room temperature. The supernatant was removed and the product was further washed with a washing solution. Then, 100 μl of a peroxidase-labeled goat anti-mouse antibody solution at 5 ng/ml was added and reacted for one hour at room temperature. The supernatant was removed and the product was washed with a washing solution. TMB (KPL) solution was added, 100 μl at a time, and reacted for 20 minutes at room temperature. After coloration, 100 μl 1 N sulfuric acid were added to stop the reaction. Absorbance at 450 nm was determined.

As a result, positive wells that reacted with ribosomal protein L7/L12 were detected, confirming presence of the antibody to ribosomal protein L7/L12.

Therefore, the cells in the positive wells were recovered and cultivated with HAT medium in a 24-well plastic plate. The fused medium that had been cultivated was diluted with an HT medium to a cell count of approximately 20 cells/ml and then mixed with 10⁶ six-week-old mouse thyroid cells suspended in the HT medium in a 96-well culture plate. The cells were cultivated for 2 weeks at 37° C. under the conditions of 7% CO₂. The antibody activity in the culture supernatant was determined by the aforementioned ELISA method and the cells that showed a positive reaction with ribosomal protein L7/L12 were recovered.

Furthermore, the same dilution and cloning procedure was repeated to obtain a total of 4 clones of hybridoma AMSP-l to 4.

### Example 6

Reaction of Monoclonal Antibody That Reacts with Ribosomal Protein L7/L12 Protein of Streptococcus pneumoniae with Streptococcus pneumoniae and Other Microorganisms.

A monoclonal antibody was produced and recovered in accordance with standard methods using the positive hybridoma cells obtained as previously described.

Specifically, cells subcultivated in RPMI 1640 medium (containing 10% FCS) was diluted with a serum-free medium to about 2×10⁵ cells/ml, 3.3×10⁵ cells/ml, and 5×10⁵ cells/ml in 25 cm² culture flasks, and the total volume was made 5 ml. After cells were grown for 3 to 5 days in 7% CO₂ at 37° C., a flask which contains the least number of original cells was selected among flasks in which cells were grown. The, same procedure was repeated until the cells diluted to 2×10⁵ cells/ml grow to 2×10⁶ cells/ml in 3 to 4 days, thereby acclimatizing the cells with the serum-free medium. Next, cloning was performed in a 96-well plate for bacteria cultivation to select cells exhibiting fastest growth and a highest antibody titer. The selected cells were grown in a 24-well plate and diluted with a serum-free medium in a 25 cm² culture flask to a concentration of about 2×10⁵ cells/ml and the total volume was made 10 ml. After incubation for 3 to 4 days in 7% CO₂ at 37° C. to a concentration of 1×10⁶ cells/ml, the culture broth was transferred to a bottle for mass cultivation together with 100 ml of 1×10⁶ cells/ml cells which were grown in the same manner in a 75 cm² flask. 100 ml of a serum-free medium was added to the mixture, which was incubated at 37° C. for two days while stirring. 200 ml of the serum-free medium was added again and the mixture was incubated for a further two days. The culture broth was divided into four aliquot, the serum-free medium was added to each portion, followed by incubation for two days. After further addition of 400 ml of the serum-free medium, the culture broth was incubated for 6 days. The culture broth was collected and centrifuged at 10,000 rpm for 15 minutes to obtain a culture supernatant including the target antibody. After the addition of 0.1% sodium azide, the culture supernatant was stored at 4° C. 100 ml of the solution containing the antibody that was obtained was diluted to a 5-fold with PBS and adsorbed in a Protein G column (5 ml bed, Pharmacia) and rinsed with PBS at 3-times the bed volume. Then elution with citrate buffer at a pH of 3 was performed. The antibody fraction was recovered and monoclonal antibody that produced each hybridoma was obtained. The monoclonal antibodies originating from the four hybridoma strains were evaluated according to the OIA method described in Publication of the translation of International Patent Application Japanese Laid-open (Toku-Hyou) No. 509565/1995.

Specifically, the OIA method comprises preparing a reactive substrate by reacting an antibody for capture on a silicon wafer having a thin film layer of silicon nitride, causing this substrate to react with an antigen which is an extract of microorganisms for a prescribed period of time, causing the captured antigen to react with an antibody (an amplification reagent) which is an enzyme-labeled antibody, and finally adding a substrate solution to produce a thin-film precipitate. The antigen-antibody reaction can be judged visually by a degree of light interference color produced in the precipitate.

The monoclonal antibody preparation was used and evaluated as a capture antibody to be immobilized on a silicon wafer having a silicon nitride thin film layer in the OIA method. Moreover, peroxidase-labeled AMGC-1 monoclonal antibody which can non-specifically react with ribosomal proteins L7/L12 protein of a variety of microorganisms described in Reference Example was used as the detect antibody. That is, enzyme labeling was performed in accordance with the method in “Analytical Biochemistry” 132 (1983), 68-73 with the reagent S-acetylthioacetic acid N-hydroxysuccinimide for binding using horseradish peroxidase (Sigma Grade VI). In the OIA reaction, monoclonal antibody in a PBS containing 0.05% sodium azide was diluted with 0.1 M HEPES (pH 8.0) to a concentration of 10 μg/ml and added onto a silicone wafer which has a thin film layer of silicon nitride, 50 μl at a time, to react for 30 minutes at room temperature, followed by washing with distilled water and use. 15 μl of antigen solution, which had been obtained by adding 0.5% Triton X-100 to culture solutions of various species of microorganisms and then extracting the solution for 5 minutes at room temperature, was added to the specimen obtained in the above-described procedure and reacted for 10 minutes at room temperature. Then, 15 μl of 20 μg/ml peroxidase-labeled AMGC1 anti-body was added and reacted for 10 minutes. After washing with distilled water, a substrate solution (KPL) was added, 15 μl at a time, and reacted for 5 minutes at room temperature. The product was washed with distilled water to judge the concentration of detection signals as an intensity of light interference by necked eyes.

As a result, as shown in Table 2 it is clear that when monoclonal antibody derived from hybridoma AMSP-2 was used as the capture antibody, all strains of Streptococcus pneumoniae tested were detected at a sensitivity of 10⁶ bacteria/ml, while reactivity of other bacteria could not be detected at a higher concentration of 10⁸ bacteria/ml. Thus, the antibody with specific reactivity to Streptococcus pneumoniae was confirmed to have been obtained by using the monoclonal antibody to ribosomal protein L7/L12.

The hybridoma AMSP-2 which produces the monoclonal antibody specific to Streptococcus pneumoniae has been deposited with National Institute of Bioscience and Human-Technology, the Agency of Industrial Science and Technology, the Ministry of International Trade and Industry, Japan, on Jul. 28, 1999, with the disposition number FERM BP-6870.

TABLE 2

Results of Detection
(10⁶ cells/ml)

| | | |
|---|---|---|
| Streptococcus pneumoniae | ATCC27336 | + |
| Streptococcus pneumoniae | IID554 | + |
| Streptococcus pneumoniae | IID555 | + |
| Streptococcus pneumoniae | IID556 | + |
| Streptococcus pneumoniae | IID557 | + |
| Streptococcus pneumoniae | IID558 | + |
| Streptococcus pneumoniae | IID559 | + |
| Streptococcus pneumoniae | IID1603 | + |

Results of Detection
(10⁸ cells/ml)

| | | |
|---|---|---|
| Escherichia coli | ATCC25922 | − |
| Enterococcus faecalis | ATCC19433 | − |
| Haemophilus influenzae | ATCC10211 | − |
| Klebsiella pneumoniae | ATCC13883 | − |
| Neisseria meningitides | IID821 | − |
| Neisseria lactamica | ATCC23970 | − |
| Neisseria meningitidis | ATCC13090 | − |
| Pseudomonas aeruginosa | ATCC27853 | − |
| GroupB streptococcus | ATCC12386 | − |
| Staphylococcus aureus | ATCC25923 | − |
| Streptococcus pyogenes | ATCC19615 | − |

(+: Positive; −: Negative)

### Example 7

Cloning of Ribosomal Protein L7/L12 Genes from Neisseria gonorrhoeae, Mass Expression in Escherichia coli and Purification of the Same Protein and Preparation of Monoclonal Antibody to the Same Protein

After inoculating an appropriate amount of Neisseria gonorrhoeae strain IID821 (obtained from. Tokyo University School of Medicine Laboratories) in a chocolate agar culture medium, the strain was cultivated for 24 hours in a CO₂ incubator under conditions of 37° C. and 0.5% CO₂. The colonies that grew were suspended in a TE buffer to a final concentration of approximately 5×10 CFU/ml. Approximately 1.5 ml of this suspension was transferred to a microcentrifugation tube and centrifuged for 2 minutes at 10,000 rpm. The supernatant was discarded. The sediment was resuspended in 567 μl TE buffer. Then 30 μl 10% SDS and 3 μl 20 mg/ml Proteinase K solution were added and thoroughly mixed. The suspension was incubated for another hour at 37° C.

Next, after adding 80 μl 10% cetyl trimethyl ammonium bromide/0.7 M NaCl solution and thoroughly mixing the product, it was incubated for 10 minutes at 65° C. Next, 700 μl chloroform-isoamyl alcohol solution at a volume ratio of 24/1 was added and stirred well. The solution was centrifuged for 5 minutes (while being kept at 4° C.) at 12,000 rpm using a microcentrifugation device and the aqueous fraction was transferred to a new microtube. Isopropanol was added to the fraction at 0.6-times its volume and the tube was vigorously shaken to form sediment of the DNA. The white DNA sediment was scooped with a glass rod and transferred to a different microcentrifugation tube containing 1 ml 70% ethanol (cooled to −20° C.).

Next, the product was centrifuged for 5 minutes at 10,000 rpm and the supernatant was gently removed. Then another 1 ml 70% ethanol was added and the product was centrifuged for 5 more minutes.

Once the supernatant had been removed, the sediment was dissolved in 100 μl TE buffer to obtain the DNA solution. The concentration of the genomic DNA solution was determined quantitatively in accordance with E5: Sectrophotometric determination of the amount of DNA or RNA, “Molecular cloning: A laboratory manual,” 1989, Eds. Sambrook, J., Fritsch, E. F., and Maniatis, T., Cold Spring Harbor Laboratory Press.

PCR was performed using 10 ng of this genomic DNA. PCR was performed using Taq polymerase (Takara Co., Ltd., code R001A). Then, 5 μl of a buffer attached to enzyme, 4 μl of a dNTP mixture attached to enzyme, and 200 pmol each of synthetic oligonucleotide E shown in SEQ ID NO:15 of the Sequence Table and synthetic oligonucleotide F shown in SEQ ID NO:16 of the Sequence Table, which were designed based on the ribosomal protein L7/L12 DNA sequence of Neisseria gonorrhoeae acquired from Internet Information (Oklahoma University, N. Gonorrhoeae Genome Project, disclosed genomic DNA data) because of the similarity with ribosomal protein L7/L12 DNA sequence of other bacteria, were added to the enzyme to bring the final volume to 50 μl.

This mixture was cycled 5 times with a TaKaRa PCR Thermal Cycler 480 for 1 minute at 95° C., 2 minutes at 50° C., and 3 minutes at 72° C. and was then cycled 25 times for 1 minute at 95° C., 2 minutes at 60° C., and 3 minutes at 72° C. Electrophoresis was performed in 1.5% agarose gel using some of this PCR product. This product was then stained with ethidium bromide (Nihon Gene Co., ltd.) and observed under ultraviolet rays to confirm amplification of approximately 400 bp cDNA. After digestion treatment with restriction endonucleases BamHI and Xhol, electrophoresis was performed in 1.5% agarose gel and staining with ethidium bromide was carried out. An approximately 370 bp band was cut out from the gel. This band was purified with Suprec01 (Takara Co., Ltd.) and then inserted into pGEX-4T-1 (Pharmacia), which is a commercial vector. Actually, vector pGEX-4T-1 and the previous DNA were mixed together at a molar ratio of 1:3 and DNA was inserted into the vector with T4 DNA ligase (Invitrogen Co.). Vector pGEX-4P-l into which DNA had been inserted was genetically introduced to Escherichia coli One-Shot Competent Cells (Invitrogen Co., Ltd.) and then inoculated in a plate of L-broth (Takara Co., Ltd.) semi-sold culture plate containing 50 μg/ml ampicillin (Sigma). The plate was then set aside at 37° C. for 12 hours and the colonies that grew were selected at random and inoculated into 2 ml L-Broth liquid culture medium containing the same concentration of ampicillin. Shake cultivation was performed at 37° C. for 8 hours and the bacteria was recovered and the plasmid was separated using Wizard Miniprep in accordance with the attached literature. The plasmid was cleaved with restriction endonuclease BamHI/XhoI. Insertion of said PCR product was confirmed by cutting out approximately 370 bp DNA. The base sequence of the DNA that had been inserted was determined using said clone.

Determination of the base sequence of the inserted DNA fragment was performed using the Fluorescence Sequencer of Applied Biosystems. The sequence sample was prepared using PRISM, Ready Reaction Dye Terminator Cycle Sequencing Kit (Applied Biosystems). First, 9.5 μl reaction stock solution, 4.0 μl T7 promoter primer at 0.8 pmol/pl (Gibco BRL) and 6.5 Al template DNA for sequencing at 0.16 μg/μl were added to a microtube with a capacity of 0.5 ml and mixed. After superposing with 100 Al mineral oil, PCR amplification was performed for 25 cycles, where one cycle consisted of 30 seconds at 96° C., 15 seconds at 55° C., and 4 minutes at 60° C. The product was then kept at 4° C. for 4 minutes. After the reaction was completed, 80 μl sterilized pure water was added and stirred. The product was centrifuged and the aqueous layer was extracted 3 times with phenol-chloroform. Ten microliters 3M sodium acetate (pH 5.2) and 300 μl ethanol were added to 100 μl aqueous layer and stirred. The product was then centrifuged for 15 minutes at room temperature and 1400 rpm and the sediment was recovered. Once the sediment was washed with 75% ethanol, it was dried under a vacuum for 2 minutes to obtain the sequencing sample. The sequencing sample was dissolved in formamide containing 4 μl 10 mM EDTA and denatured for 2 minutes at 90° C. This was then cooled in ice and submitted to sequencing. One of the 5 clones obtained had homology of the sequence with the probe used for PCR. In addition, DNA sequences extremely similar to the gene sequence of ribosomal protein L7/L12 gene of the other microorganisms, for example, Haemophilus influenzae, were discovered. The entire base sequence and the corresponding amino acid sequence of the structural gene moiety are as shown in SEQ ID NO:21 and SEQ ID NO: 22 of the Sequence Table. This gene fragment clearly codes for ribosomal protein L7/L12 gene of Neisseria gonorrhoeae.

Neisseria gonorrhoeae fusion GST ribosome protein L7/L12 protein prepared by the same method as in Example 2 was obtained using the Neisseria gonorrhoeae fusion GST ribosomal protein L7/L12 protein expression vector constructed in this way.

Furthermore, hybridoma strain GCRB-3, which produces monoclonal antibody to ribosomal protein L7/L12 of Neisseria gonorrhoeae, was obtained in accordance with the method in Example 3.

### Example 8

Reaction of Monoclonal Antibody That Reacts with Ribosomal Protein L7/L12 Protein of Neisseria gonorrhoeae with Neisseria gonorrhoeae and Other Microorganism

Monoclonal antibody was produced and recovered in accordance with standard methods using the positive hybridoma cells GCRB-3 obtained as previously described.

Basically, 5×10⁶ cells (in PBS) that had been subcultivated using RPMI 1640 culture medium (containing 10% FCS) were intraperitoneally injected into Balb/C mice that had been intraperitoneally injected with 0,5 ml Pristane 2 weeks earlier. Ascites was recovered 3 weeks later and the centrifugation supernatant was obtained.

The solution containing antibody that was obtained was adsorbed in a Protein A column (5 ml bed, Pharmacia) and rinsed with PBS at 3-times the bed volume. Then elution with citrate buffer at a pH of 3 was performed. The antibody fraction was recovered and the monoclonal antibody that was produced by each hybridoma was obtained. The monoclonal antibody derived from the GCRB-3 hybridoma was used in ELISA.

The sandwich assay method was used to assess the monoclonal antibody. The monoclonal antibody that was prepared was used as an enzyme-labeled antibody by being chemically bound to peroxidase. That is, enzyme labeling was performed in accordance with the method in “Analytical Biochemistry” 132 (1983), 68-73 with the reagent S-acetylthioacetic acid N-hydroxysuccinimide for binding using horseradish peroxidase (Sigma Grade VI). By means of the ELISA reaction, a solution of commercial anti-Neisseria gonorrhoeae polyclonal antibody in PBS containing 0.05% sodium azide (Virostat, rabbit) was diluted to a concentration of 10 μg/ml and poured, 100 μl at a time, into a separate 96-well plate and adsorbed overnight at 4° C.

After removal of the supernatant, 200 μl 1% bovine serum albumin solution (in PBS) were added and reacted for 1 hour and blocked at room temperature. The supernatant was removed and the product was washed with washing solution (0.02% Tween 20, PBS). One-hundred microliters of antigen solution, which had been obtained by adding Triton X-100 to culture solutions of each species of microorganisms to a concentration of 0.3% and then extracting the solution for 5 minutes at room temperature, were added to this and reacted for 2 hours at room temperature. The supernatant was removed and the product was further washed with washing solution. Then 100 μl peroxidase-labeled anti-ribosomal protein L7/L12 antibody solution at 5 μg/ml were added and reacted for 1 hour at room temperature. The supernatant was removed and the product was washed with washing solution. TMB (KPL) solution was added, 100 μl at a time, and reacted for 20 minutes at room temperature. After coloration, 100 μl 1 N sulfuric acid were added to stop the reaction. Absorbance at 450 nm was determined.

As a result, as shown in Table 3 it is clear that when monoclonal antibody derived from hybridoma GCRB-3 was used as the enzyme-labeled antibody, all strains of Neisseria gonorrhoeae tested were detected at a sensitivity of 10⁶ cells/ml, while reactivity of other species belonging to the genus Neisseria and other microorganisms could not be detected, even at high concentrations of 10⁸ cells/ml and therefore, antibody with specific reactivity to Neisseria gonorrhoeae can be obtained by using monoclonal antibody to ribosomal protein L7/L12 protein.

TABLE 3

Results of Detection
(10⁶ cells/ml)

| | | |
|---|---|---|
| Neisseria gonorrhoeae | ATCC9793 | + |
| Neisseria gonorrhoeae | ATCC19424 | + |
| Neisseria gonorrhoeae | ATCC27628 | + |
| Neisseria gonorrhoeae | ATCC27629 | + |
| Neisseria gonorrhoeae | ATCC27630 | + |
| Neisseria gonorrhoeae | ATCC27631 | + |
| Neisseria gonorrhoeae | ATCC27632 | + |
| Neisseria gonorrhoeae | ATCC27633 | + |
| Neisseria gonorrhoeae | ATCC35541 | + |
| Neisseria gonorrhoeae | ATCC35542 | + |
| Neisseria gonorrhoeae | ATCC43069 | + |
| Neisseria gonorrhoeae | ATCC43070 | + |
| Neisseria gonorrhoeae | ATCC49226 | + |

Results of Detection
(10⁸ cells/ml)

| | | |
|---|---|---|
| Escherichia coli | ATCC25922 | − |
| Enterococcus faecalis | ATCC19433 | − |
| Haemophilus influenzae | ATCC10211 | − |
| Klebsiella pneumoniae | ATCC13883 | − |
| Neisseria gonorrhoeae | IID821 | − |
| Neisseria lactamica | ATCC23970 | − |
| Neisseria meningitidis | ATCC13090 | − |
| Pseudomonas aeruginosa | ATCC27853 | − |
| GroupB streptococcus | ATCC12386 | − |
| Stauphylococcus aureus | ATCC25923 | − |
| Streptococcus pyogenes | ATCC19615 | − |

(+: Positive; −: Negative)

### Example 9

Acquisition of Monoclonal Antibody to Ant-Ribosomal Protein L7/L12 Protein with Specificity to Genus Neisseria

After inoculating an appropriate amount of Neisseria gonorrhoeae strain IID821 (obtained from Tokyo University School of Medicine Laboratories) in a chocolate agar culture medium, the strain was cultivated for 24 hours in a CO₂ incubator under conditions of 37° C. and 0.5% CO₂. The colonies that grew were suspended in a TE buffer to a final concentration of approximately 5×10⁹ CFU/ml. Approximately 1.5 ml of this suspension was transferred to a microcentrifugation tube and centrifuged for 2 minutes at 10,000 rpm. The supernatant was discarded. The sediment was resuspended in 567 μl TE buffer. Then 30 μl 10% SDS and 3 μl 20 mg/ml Proteinase K solution were added and thoroughly mixed. The suspension was incubated for another hour at 37° C. Next, after adding 80 μl 10% cetyl trimethyl ammonium bromide/0.7 M NaCl solution and thoroughly mixing the product, it was incubated for 10 minutes at 65° C. Next, 700 μl chloroform-isoamyl alcohol solution at a volume ratio of 24/1 was added and stirred well.

The solution was centrifuged for 5 minutes (while being kept at 4° C.) at 12,000 rpm using a microcentrifugation device and the aqueous fraction was transferred to a new microtube. Isopropanol was added to the fraction at 0.6-times its volume and the tube was vigorously shaken to form sediment of the DNA. The white DNA sediment was scooped with a glass rod and transferred to a different microcentrifugation tube containing 1 ml 70% ethanol (cooled to −20° C.).

Next, the product was centrifuged for 5 minutes at 10,000 rpm and the supernatant was gently removed. Then another 1 ml 70% ethanol was added and the product was centrifuged for 5 more minutes. Once the supernatant had been removed, the sediment was dissolved in 100 μl TE buffer to obtain the DNA solution. The concentration of the genomic DNA solution was determined quantitatively in accordance with E5: Sectrophotometric determination of the amount of DNA or RNA, “Molecular cloning: A laboratory manual,” 1989, Eds. Sambrook, J., Fritsch, E. F., and Maniatis, T., Cold Spring Harbor Laboratory Press.

PCR was performed using 10 ng of this genomic DNA. Taq polymerase (Takara Co., Ltd., code R001A) was employed for PCR. Then, 5 μl buffer attached to enzyme, 4 μl dNTP mixture attached to enzyme, and 200 pmol each of synthetic oligonucleotide E shown in SEQ ID NO:15 of the Sequence Table and synthetic oligonucleotide F shown in SEQ ID NO:16 of the Sequence Table, which were designed based on the ribosomal protein L12/L12 DNA sequence of Neisseria gonorrhoeae acquired from Internet information (Oklahoma University, N. Gonorrhoeae Genome Project, disclosed genome DNA data) because of the similarity with ribosomal protein L12/L12 DNA sequence of other bacteria, were added to the enzyme to bring the final volume to 50 μl.

This mixture was cycled 5 times with a TaKaRa PCR Thermal Cycler 480 for 1 minute at 95° C., 2 minutes at 50° C., and 3 minutes at 72° C. and was then cycled 25 times for 1 minute at 95° C., 2 minutes at 60° C., and 3 minutes at 72° C. Electrophoresis was performed in 1.5% agarose gel using some of this PCR product. This product was then stained with ethidium bromide (Nihon Gene Co., ltd.) and observed under ultraviolet rays to confirm amplification of approximately 400 bp cDNA. After digestion treatment with restriction endonucleases BamHI and Xhol, electrophoresis was performed in 1.5% agarose gel and staining with ethidium bromide was carried out. An approximately 370 bp band was cut out from the gel. This band was purified with Suprec01 (Takara Co., Ltd.) and then inserted into pGEX-6P-1 (Pharmacia), which is a commercial vector. This same vector can function as an expression vector for the desired molecule, which can express fused protein with GST protein, by insertion of the desired gene fragment into the appropriate restriction endonuclease site. Actually, vector pGEX-6P-1 and the previous DNA were mixed together at a molar ratio of 1:5 and DNA was inserted into the vector with T4 DNA ligase (Invitrogen Co.). Vector pGEX-6P-1 into which DNA had been inserted was genetically introduced to Escherichia coli One-Shot Competent Cells (Invitrogen Co., Ltd.) and then inoculated in a plate of L-broth (Tomisaka Co., ltd.) semi-sold culture plate containing 50 μg/ml ampicillin (Sigma). The plate was then set aside at 37° C. for 12 hours and the colonies that grew were selected at random and inoculated into 2 ml L-Broth liquid culture medium containing the same concentration of ampicillin. Shake cultivation was performed at 37° C. for 8 hours and the bacteria was recovered and the plasmid was separated using Wizard Miniprep in accordance with the attached literature. The plasmid was cleaved with restriction endonuclease BamHI/XhoI. Insertion of said PCR product was confirmed by cutting out approximately 370 bp DNA. The base sequence of the DNA that had been inserted was determined using said clone.

Determination of the base sequence of the inserted DNA fragment was performed using the Fluorescence Sequencer of Applied Biosystems. The sequence sample was prepared using PRISM, Ready Reaction Dye Terminator Cycle Sequencing Kit (Applied Biosystems). First, 9.5 μl reaction stock solution, 4.0 μl T7 promoter primer at 0.8 pmol/pl (Gibco BRL) and 6.5 μl template DNA for sequencing at 0.16 μg/μl were added to a microtube with a capacity of 0.5 ml and mixed. After superposing with 100 μl mineral oil, PCR amplification was performed for 25 cycles, where one cycle consisted of 30 seconds at 96° C., 15 seconds at 55° C., and 4 minutes at 60° C. The product was then kept at 4° C. for 4 minutes. After the reaction was completed, 80 μl sterilized pure water was added and stirred. The product was centrifuged and the aqueous layer was extracted 3 times with phenol-chloroform. Ten microliters 3M sodium acetate (pH 5.2) and 300 μl ethanol were added to 100 μl the aqueous layer and stirred. The product was then centrifuged for 15 minutes at room temperature and 1400 rpm and the sediment was recovered. Once the sediment was washed with 75% ethanol, it was dried under a vacuum for 2 minutes to obtain the sequencing sample. The sequencing sample was dissolved in formamide containing 4 μl 10 mM EDTA and denatured for 2 minutes at 90° C. This was then cooled in ice and submitted to sequencing.

One of the 4 clones obtained had homology of the sequence with the probe used for PCR. In addition, DNA sequences extremely similar to the gene sequence of ribosomal protein L7/L12 gene of the other microorganisms, for example, Haemophilus influenzae, were discovered. The entire base sequence and the corresponding amino acid sequence of the structural gene moiety are as shown in Sequence ID No. 21 and SEQ ID NO: 22 of the Sequence Table. This gene fragment clearly codes for Neisseria gonorrhoeae ribosomal protein L7/L12 protein.

50 ml Escherichia coli into which expression vector had been inserted was cultivated overnight in a two-fold concentration YT medium at 37° C. Then, 450 ml of the two-fold concentration YT medium was heated at 37° C. for 1 hour. 50 ml of the Escherichia coli solution that had been cultivated overnight was introduced to 450 ml of the aforementioned medium. After cultivation for one hour at 37° C., 100 μl 500 mM IPTG was introduced and cultivated for 4 hours at 25° C. The product was then recovered and introduced 250 ml to a centrifugation tube and centrifuged for 20 minutes at 5000 rpm. The supernatant was discarded and dissolved in 25 ml each 50 mM Tris-HCl at a pH of 7.4 and Lysis buffer containing 25% sucrose.

Furthermore, 1.25 ml 10% NP-40 and 125 μl 1 M MgCl₂ were added and transferred to a plastic tube. Sonication was performed 1 minute×5 times while ice cold. The product was centrifuged for 15 minutes at 12,000 rpm and the supernatant was recovered.

Next, the aforementioned supernatant was adsorbed on a glutathione sepharose column (manufactured by Pharmacia) conditioned with PBS. Then, the column was washed with PBS three times the bed volume. Elution was performed with 50 mM Tris-HCl at a pH of 8.0 containing 10 mM glutathione. The protein content in the fraction was determined by the pigment bonding method (Bradford method; Biorad Co.) and the main fraction was acquired. The main fraction was dialyzed three times against 3L PBS. 1 ml of a cleavage buffer containing 500 mM Tris-HCl (pH 7.0), 1.5 M NaCl, 10 mM EDTA, and 10 mM DTT was added to 10 ml of 1 mg/ml solution of the resulting GST fusion ribosomal protein L7/L12 protein. 100 μl of 2 u/μl PreScission Protease (manufactured by Pharmacia company) was further added and reacted at 4° C. to separate the GST moiety from ribosomal protein L7/L12 protein.

The reaction solution was caused to pass through a glutathione sepharose column which had been conditioned with PBS. The solution coming out from the column was recovered. Onebed volume of PBS was caused to pass through and also recovered. Purity of the purified ribosomal protein L7/L12 that was obtained was confirmed by electrophoresis to be approximately 90%, showing that a purity satisfactory for an immunogen could be guaranteed.

First, 100 μg protein antigen of ribosomal protein L7/L12 protein of Neisseria gonorrhoeae was dissolved in 200 μl PBS and then 200 μl Freund's complete adjuvant was added and mixed and emulsification was performed. 200 μl was intraperitoneally injected to immunize mice. Then, the same emulsion antigen was intraperitoneally injected after 2 weeks, after 4 weeks, and after 6 weeks. A two-fold concentration antigen emulsion was further injected intraperitoneally after 10 weeks and after 14 weeks. The spleen was excised 3 days after the final immunization and submitted to cell fusion.

After thoroughly mixing 2×10⁷ myeloma cells per 10⁸ spleen cells from mice, which had been recovered aseptically, in a glass tube, the mixture was centrifuged for 5 minutes at 1500 rpm and the supernatant was discarded. The cells were thoroughly mixed.

The myeloma cells used for cell fusion were obtained by cultivation of cell strain NS-1 with an RPMI 1640 culture medium containing 10% FCS, cultivating this product beginning 2 weeks before cell fusion using an RPMI 1640 medium containing 0.13 mM azaguanine, 0.5 μg/ml MC-210, and 10% FCS for 1 weeks, and then further cultivating the cell strain for 1 week with an RPMI 1640 medium containing 10% FCS. 30 ml of 50 ml RPMI 1640 culture medium that had been kept at 37° C. was added to the mixed cell sample and centrifuged at 1,500 rpm. After removal of the supernatant, 1 ml 50% polyethylene glycol that had been kept at 37° C. was added and stirred for 2 minute. 10 ml RPMI 1640 medium kept at 37° C. was added and the solution was vigorously mixed for approximately 5 minutes as it was suctioned and evacuated from a sterile pipette.

After centrifugation for 5 minutes at 1,000 rpm and removal of the supernatant, 30 ml HAT culture medium were added to bring the cell concentration to 5×10⁶ cells/ml. This mixture was stirred till uniform and then poured, 0, 1 ml at a time, into a 96-well culture plate and cultivated at 37° C. under 7% CO₂. HAT medium was added, 0.1 ml at a time, on Day 1 and at Week 1 and Week 2.

Then the cells that had produced the desired antibody were assessed by ELISA. Solutions of ribosomal protein L7/L12 protein of Neisseria gonorrhoeae dissolved in PBS containing 0.05% sodium azide were diluted to 10 μg/ml and were poured, 100 pl at a time, into separate 96-well plates and adsorbed overnight at 4° C. After removal of the supernatant, 200 μl 1% FCS solution (in PBS) were added and reacted and blocked for 1 hour at room temperature. The supernatant was removed and the product was washed with a washing solution (0.02% Tween 20, PBS). 100 μl of a culture solution of fusion cells was added and reacted for two hours at room temperature. The supernatant was removed and the product was further washed with a washing solution. Then, 100 μl of a peroxidase-labeled goat anti-mouse antibody solution at 5 ng/ml was added and reacted for one hour at room temperature. The supernatant was removed and the product was washed with a washing solution. TMB (KPL) solution was added, 100 μl at a time, and reacted for 20 minutes at room temperature. After coloration, 100 μl 1 N sulfuric acid were added to stop the reaction. Absorbance at 450 nm was determined.

As a result, positive wells that reacted with ribosomal protein L7/L12 protein were detected, confirming presence of the antibody to ribosomal protein L7/L12.

Therefore, the cells in the positive wells were recovered and cultivated with HAT medium in a 24-well plastic plate. The fused medium that had been cultivated was diluted with an HT medium to a cell count of approximately 20 cells/ml. Then 50 μl the medium was mixed with 10⁶ six-week-old mouse thyroid cells suspended in the HT medium in a 96-well culture plate. The cells were cultivated for 2 weeks at 37° C. under the conditions of 7% C02. The antibody activity in the culture supernatant was determined by the aforementioned ELISA method and the cells that showed a positive reaction with ribosomal protein L7/L12 were recovered.

Furthermore, the same dilution and cloning procedure was repeated to obtain a total of 4 clones of hybridoma AMGC-5 to AMGC-8.

Monoclonal antibody was produced and recovered in accordance with standard methods using the positive hybridoma cells obtained as previously described.

Specifically, cells subcultivated in RPMI 1640 medium (containing 10% FCS) was diluted with a serum-free medium to about 2×10⁵ cells/ml, 3.3×10⁵ cells/ml, and 5×10⁵ cells/ml in 25 cm² culture flask, and the total was made 5 ml. After cells were grown for 3 to 5 days in 7% C02 at 37° C., a flask which contains the least number of original cells was selected among flasks in which cells were grown. The same procedure was repeated until the cells diluted to 2×10⁵ cells/ml grow to 2×10⁶ cells/ml in 3 to 4 days, thereby acclimatizing the cells with the serum-free medium. Next, cloning was performed in a 96-well plate for bacteria cultivation to select cells exhibiting fastest growth and a highest antibody titer. The selected cells were grown in a 24-well plate and diluted with a serum-free medium in a 25 cm² culture flask to a concentration of about 2×10⁵ cells/ml and the total volume was made 10 ml. After incubation for 3 to 4 days in 7% CO₂ at 37° C. to a concentration of 1×10⁶ cells/ml, the culture broth was transferred to a bottle for mass cultivation together with 100 ml of 1×10⁶ cells/ml cells which were grown in the same manner in a 75 cm² flask. 100 ml of a serum-free medium was added to the mixture, which was incubated at 37° C. for two days while stirring. 200 ml of the serum-free medium was added again and the mixture was incubated for a further two days. The culture broth was divided into four aliquot, the serum-free medium was added to each portion, followed by incubation for two days. After further addition of 400 ml of the serum-free medium, the culture broth was incubated for 6 days. The culture broth was collected and centrifuged at 10,000 rpm for 15 minutes to obtain a culture supernatant including the target antibody. After the addition of 0.1% sodium azide, the culture supernatant was stored at 4° C. 100 ml of the solution containing the antibody that was obtained was diluted to a 5-fold with PBS and adsorbed in a protein G column (5 ml bed, Pharmacia) and rinsed with PBS at 3-times the bed volume. Then elution with citrate buffer at a pH of 3 was performed. The antibody fraction was recovered and monoclonal antibody that produced each hybridoma was obtained.

The monoclonal antibodies originating from the four hybridoma strains were evaluated according to the OIA method described in International Patent Application Japanese Laid-open No. 509565/1995.

Specifically, the OIA method comprises preparing a reactive substrate by reacting an antibody for capture on a silicon wafer having a thin film layer of silicon nitride, causing this substrate to react with an antigen which is an extract of microorganisms for a prescribed period of time, causing the captured antigen to react with an antibody (an amplification reagent) which is an enzyme-labeled antibody, and finally adding a substrate solution to produce a thin-film precipitate. The antigen-antibody reaction can be judged visually by a degree of light interference color produced in the precipitate.

The monoclonal antibody preparation was used as a capture antibody to be immobilized on a silicon wafer having a silicon nitride thin film layer in the OIA method. Moreover, peroxidase-labeled AMGC-1 monoclonal antibody which can non-specifically react with ribosomal proteins L7/L12 protein of a variety of microorganisms described in Reference Example was used as the detecte reagent antibody. That is, enzyme labeling was performed in accordance with the method in “Analytical Biochemistry” 132 (1983), 68-73 with the reagent S-acetylthioacetic acid N-hydroxysuccinimide for binding using horseradish peroxidase (Sigma Grade VI).

In the OIA reaction, monoclonal antibody in a PBS containing 0.05% sodium azide was diluted with 0.1 M HEPES (pH 8.0) to a concentration of 10 μg/ml and added onto a silicone wafer which has a thin film layer of silicon nitride, 50 μl at a time, to react for 30 minutes at room temperature, followed by washing with distilled water and use. 15 μl of antigen solution, which had been obtained by adding 0.5% Triton X-100 to culture suspension of various species of microorganisms and then extracting the suspension for 5 minutes at room temperature, was added to the specimen obtained in the above-described procedure and reacted for 10 minutes at room temperature. Then, 15 μl of 20 μg/ml peroxidase-labeled AMGC1 was added and reacted for 10 minutes. After washing with distilled water, a substrate solution (KPL Co.) was added, 15 μl at a time, and reacted for 5 minutes at room temperature. The product was washed with distilled water to judge the concentration of detection signals as an intensity of light interference by necked eyes.

As a result, as shown in Table 4 it is clear that when monoclonal antibody derived from hybridoma AMGC-8 was used as the capture antibody, all strains of Neisseria genus tested were detected at a sensitivity of 10⁸ cells/ml, while reactivity of other microorganisms could not be detected. Thus, the antibody with specific reactivity to Neisseria genus was confirmed to have been obtained by using the monoclonal antibody to ribosomal protein L7/L12 protein.

TABLE 4

Results of Detection
(10⁶ cells/ml)

| | | |
|---|---|---|
| Neisseria gonorrhoeae | IID821 | + |
| Neisseria lactamica | ATCC23970 | + |
| Neisseria meningitidis | ATCC13090 | + |
| Escherichia coli | ATCC25922 | − |
| Enterococcus faecalis | ATCC19433 | − |
| Haemophiius influenzae | ATCC10211 | − |
| Klebsiella pneumoniae | ATCC13883 | − |
| Pseudomonas aeruginosa | ATCC27853 | − |
| GroupB streptococcus | ATCC12386 | − |
| Stauphylococcus aureus | ATCC25923 | − |
| Streptococcus pneumoniae | ATCC27336 | − |
| Streptococcus pyogenes | ATCC19615 | − |

(+: Positive; −: Negative)

### Example 10

Acquisition of a Polyclonal Antibody Which Specifically Reacts with Ribosomal Protein L7/L12 Protein of Haemophilus influenzae Using a Ribosomal Protein L7/L12Protein-Immobilized Affinity Column

A centrifugal supernatant of Haemophilus influenzae cell extract which had been treated with 0.5% Triton X-100 was used as an antigen. About 1.2 ml of a physiological saline solution containing 100 μg of antigen was emulsified with the addition of 1.5 ml of Freund's adjuvant. The emulsion was subcutaneously injected into four SPF Japanese White Rabbits to immunize the animals. The rabbits were immunized 5 to 6 times, once every two weeks, and the antibody titer was confirmed.

The antibody titer was confirmed by the ELISA method. Solutions of ribosomal protein L7/L12 protein of Haemophilus influenzae dissolved in PBS containing 0.05% sodium azide diluted to 10 μg/ml were poured, 100 μl at a time, into 96-well plates and adsorbed overnight at 4° C. After removal of the supernatant, 200 μl 1% FCS solution (in PBS) were added and reacted and blocked for 1 hour at room temperature. The supernatant was removed and the product was washed with a washing solution (0.02% Tween 20, PBS). 100 μl of a solution obtained by diluting normal rabbit serum and immunized rabbit antiserum was added and reacted for two hours at room temperature. The supernatant was removed and the product was further washed with a washing solution. Then, 100 μl of a peroxidase-labeled goat anti-rabbit IgG antibody solution at 50 ng/ml was added and reacted for one hour at room temperature. The supernatant was removed and the product was washed with a washing solution. OPD solution (Sigma Co.) was added, 100 μl at a time, and reacted for 20 minutes at room temperature. After coloration, 100 μl of 1 N sulfuric acid was added to stop the reaction. Absorbance at 492 nm was determined.

After confirming that the antibody titer had increased, a large quantity of blood was collected. Blood was collected in a centrifugal tube made of glass from the ear artery, allowed to stand for one hour at 37° C., then overnight at 4° C. The mixture was centrifuged at 3000 rpm for 5 minutes and the supernatant was recovered. The resulting anti-serum was stored at 4° C.

Next, an affinity column with immobilized ribosomal protein L7/L12 protein of Haemophilus influenzae and Neisseria gonorrhoeae was prepared. HiTrap NHS-activated column (1 ml, manufactured by Pharmacia) was used. Immediately after replacing the column with 6 ml of 1 mM HCl, 1 ml of a PBS solution of ribosomal protein L7/L12 protein adjusted to 1 mg/ml was charged. The column was allowed to stand for 15 minutes at 25° C. This procedure was repeated 5 times, thereby feeding the total 5 ml of the PBS solution of ribosomal protein L7/L12 protein. Then, 6 ml of Buffer A (0.5M ethanolamine, 0.5 M NaCl, pH 8.3), 6 ml of Buffer B (0.1 M acetic acid, 0.5M NaCl, pH 4), and 6 ml of Buffer A were charged as blocking reagents. After allowing to stand for 15 minutes at 25° C., 6 ml of Buffer B, 6 ml of Buffer A, and 6 ml of Buffer B were further added. The mixture was then equilibrated with 6 ml of PBS.

Using the affinity column with immobilized ribosomal protein L7/L12 protein of Haemophilus influenzae, the polyclonal antibody in the resulting anti-serum was purified using the supernatant of Triton X-100 treated bacteria of Haemophilus influenzae as an antigen. This antiserum was first diluted with PBS to a volume of 5 times, caused to pass through a 0.45 μm filter, then caused to be adsorbed in the column immobilized with ribosomal protein L7/L12 protein of Haemophilus influenzae at a flow rate of 0.5 ml/min. After elution from the column with 0.1 M glycine (pH 2.1) and immediately neutralizing with 1 M Tris-HCl (pH 9.0), eluted fractions of the target antibody were recovered by the ELISA method, the same as anti-titer measuring method. These fractions were caused to pass through the affinity column immobilized with the ribosomal protein L7/L12 protein of Neisseria gonorrhoeae, which was equilibrated with PBS whereby the antibody that reacts with the ribosomal protein L7/L12 protein of Neisseria gonorrhoeae was adsorbed and fraction which passed through without adsorption was recovered.

The polyclonal antibody purified in this manner was evaluated by the same OIA method as in Example 6.

The purified antibody was used as a capture antibody for the OIA method. Moreover, peroxidase-labeled AMGC-l monoclonal antibody described in Reference Example was used as the detecte antibody. That is, enzyme labeling was performed in accordance with the method in “Analytical Biochemistry” 132 (1983), 68-73 with the reagent S-acetylthioacetic acid N-hydroxysuccinimide for binding using horseradish peroxidase (Sigma Grade VI). In the OIA reaction, the purified polyclonal antibody in a PBS containing 0.05% sodium azide was diluted with 0.1 M HEPES (pH 8.0) to a concentration of 10 μg/ml and added onto a silicone wafer, 50 μl at a time, to react for 30 minutes at room temperature, followed by washing with distilled water and use.

15 μl of antigen solution, which had been obtained by adding 0.5% Triton X-100 to culture suspension of various species of microorganisms and then extracting the suspension for 5 minutes at room temperature, was added to the specimen obtained in the above-described procedure and reacted for 10 minutes at room temperature. Then, 15 μl of 20 μg/ml peroxidase-labeled AMGC1 was added and reacted for 10 minutes. After washing with distilled water, a substrate solution (KPL) was added, 15 μl at a time, and reacted for 5 minutes at room temperature. The product was washed with distilled water to observe the blue color concentration by the necked eyes.

As a result, as shown in Table 5 it is clear that when the purified polyclonal antibody of APHI2-2 was used as the capture antibody, Haemophilus influenzae tested at a sensitivity of 108 bacteria/ml was detected, while reactivity of other microorganisms could not be detected. Thus, the antibody with specific reactivity to Haemophilus influenzae was confirmed to have been obtained by using the polyclonal antibody purified by an affinity column immobilized with the ribosomal protein L7/L12 protein of Haemophilus influenzae.

TABLE 5

Results of Detection
(10⁸ cells/ml)

| | | |
|---|---|---|
| Haemophilus influenzae | ATCC10211 | + |
| Escherichia coli | ATCC25922 | − |
| Enterococcus faecalis | ATCC19433 | − |
| Klebsiella pneumoniae | ATCC13883 | − |
| Neisseria gonorrhoeae | IID821 | − |
| Neisseria lactamica | ATCC23970 | − |
| Neisseria meningitidis | ATCC13090 | − |
| Pseudomonas aeruginosa | ATCC27853 | − |
| GroupB Streptococcus | ATCC12386 | − |
| Stauphylococcus aureus | ATCC25923 | − |
| Streptococcus pneumoniae | ATCC27336 | − |
| Streptococcus pyogenes | ATCC19615 | − |

(+: Positive; −: Negative)

### Reference Example 1

Acquisition of Monoclonal Antibody That Reacts Non-Specifically with Ribosomal Protein L7/L12 Protein of Various Bacteria

So called sandwich assay in which an antigen is sandwiched between a capture antibody and a labeled antibody for detection is useful to detect microorganisms by optical immunoassay and the ELISA method because of its high detection sensitivity. In this instance, not only an antibody which specifically reacts with antigens originating from the subject microorganism, but also another antibody which recognizes an antigen epitope are required.

Antibodies which react non-specifically with dribosomal protein L7/L12 protein originating various microorganisms are very useful as an antibody which can constitute a sandwich assay with an antibody which reacts with specifically ribosomal protein L7/L12 protein.

Fortunately, ribosomal proteins L7/L12 protein of a variety of microorganisms have a region wherein the amino acid sequence is homologous. Here, the inventors have been successful in acquiring a monoclonal antibody which exhibits a cross reaction with ribosomal proteins L7/L12 protein of various species of microorganisms from Neisseria gonorrhoeae. It has been discovered that an antibody to anti-ribosomal protein L7/L12 protein having no specificity, which was acquired from one species of microorganisms can be used for sandwich assay of all microorganisms.

Cloning of ribosomal protein L7/L12 genes from Neisseria gonorrhoeae, mass expression in Escherichia coli and purification of the same protein, and preparation of monoclonal antibody to the same protein were performed.

After inoculating an appropriate amount of Neisseria gonorrhoeae strain IID821 (obtained from Tokyo University School of Medicine Laboratories) in a chocolate agar culture medium, the strain was cultivated for 24 hours in a CO₂ incubator under conditions of 37° C. and 0.5% CO₂. The colonies that grew were suspended in a TE buffer to a final concentration of approximately 5×10⁹ CFU/ml. Approximately 1.5 ml of this suspension was transferred to a microcentrifugation tube and centrifuged for 2 minutes at 10,000 rpm. The supernatant was discarded. The sediment was resuspended in 567 μl TE buffer. Then 30 μl 10% SDS and 3 μl 20 mg/ml Proteinase K solution were added and thoroughly mixed. The suspension was incubated for another hour at 37° C. Next, after adding 80 μl 10% cetyl trimethyl ammonium bromide/0.7 M NaCl solution and thoroughly mixing the product, it was incubated for 10 minutes at 65° C. Next, 700 μl chloroform-isoamyl alcohol solution at a volume ratio of 24/1 was added and stirred well. The solution was centrifuged for 5 minutes (while being kept at 4° C.) at 12,000 rpm using a microcentrifugation device and the aqueous fraction was transferred to a new microtube. Isopropanol was added to the fraction at 0.6-times its volume and the tube was vigorously shaken to form sediment of the DNA. The white DNA sediment was scooped with a glass rod and transferred to a different microcentrifugation tube containing 1 ml 70% ethanol (cooled to −20° C.).

Next, the product was centrifuged for 5 minutes at 10,000 rpm and the supernatant was gently removed. Then another 1 ml 70% ethanol was added and the product was centrifuged for 5 more minutes. Once the supernatant had been removed, the sediment was dissolved in 100 μl TE buffer to obtain the DNA solution. The concentration of the genomic DNA solution was determined quantitatively in accordance with E5: Sectrophotometric determination of the amount of DNA or RNA, “Molecular cloning: A laboratory manual,” 1989, Eds. Sambrook, J., Fritsch, E. F., and Maniatis, T., Cold Spring Harbor Laboratory Press.

PCR was performed using 10 ng of this genomic DNA. Taq polymerase (Takara Co., Ltd., code R001A) was employed for PCR. Then, 5 μl of a buffer attached to enzyme, 4 μl of dNTP mixture attached to enzyme, and 200 pmol of each of synthetic oligonucleotide E shown in SEQ ID NO:15 of the Sequence Table and synthetic oligonucleotide F shown in SEQ ID NO:16 of the Sequence Table, which were designed based on the ribosomal protein L7/L12 DNA sequence of Neisseria gonorrhoeae acquired from Internet information (Oklahoma University, N. Gonorrhoeae Genome Project, disclosed genomic DNA data) because of the similarity with ribosomal protein L7/L12 DNA sequence of other bacteria, were added to the enzyme to bring the final volume to 50 μl.

This mixture was cycled 5 times with a TaKaRa PCR Thermal Cycler 480 for 1 minute at 95° C., 2 minutes at 50° C., and 3 minutes at 72° C. and was then cycled 25 times for 1 minute at 95° C., 2 minutes at 60° C., and 3 minutes at 72° C. Electrophoresis was performed in 1.5% agarose gel using some of this PCR product. This product was then stained with ethidium bromide (Nihon Gene Co., ltd.) and observed under ultraviolet rays to confirm amplification of approximately 400 bp cDNA. After digestion treatment with restriction endonucleases BamHI and Xhol, electrophoresis was performed in 1.5% agarose gel and staining with ethidium bromide was carried out. An approximately 370 bp band was cut out from the gel. This band was purified with Suprec01 (Takara Co., Ltd.) and then inserted into pGEX-4T-1 (Pharmacia), which is a commercial vector. Actually, vector pGEX-4T-1 and the previous DNA were mixed together at a molar ratio of 1:3 and DNA was inserted into the vector with T4 DNA ligase (Invitrogen Co.). Vector pGEX-4T-1 into which DNA had been inserted was genetically introduced to Escherichia coli One-Shot Competent Cells (Invitrogen Co., Ltd.) and then inoculated in a plate of L-broth (Takara Co., Ltd.) semi-sold culture plate containing 50 μg/ml ampicillin (Sigma). The plate was then set aside at 37° C. for 12 hours and the colonies that grew were selected at random and inoculated into 2 ml L-Broth liquid culture medium containing the same concentration of ampicillin. Shake cultivation was performed at 37° C. for 8 hours and the bacteria was recovered and the plasmid was separated using Wizard Miniprep in accordance with the attached literature. The plasmid was cleaved with restriction endonuclease BamHI/XhoI. Insertion of said PCR product was confirmed by cutting out approximately 370 bp DNA. The base sequence of the DNA that had been inserted was determined using said clone.

Determination of the base sequence of the inserted DNA fragment was performed using the Fluorescence Sequencer of Applied Biosystems. The sequence sample was prepared using PRISM, Ready Reaction Dye Terminator Cycle Sequencing Kit (Applied Biosystems). First, 9.5 μl reaction stock solution, 4.0 μl T7 promoter primer at 0.8 pmol/μl (Gibco BRL) and 6.5 μl template DNA for sequencing at 0.16 μg/pl were added to a microtube with a capacity of 0.5 ml and mixed. After superposition with 100 μl mineral oil, PCR amplification was performed for 25 cycles, where one cycle consisted of 30 seconds at 96° C., 15 seconds at 55° C., and 4 minutes at 60° C. The product was then kept at 4° C. for 4 minutes. After the reaction was completed, 80 μl sterilized pure water was added and stirred. The product was centrifuged and the aqueous layer was extracted 3 times with phenol-chloroform. Ten microliters 3M sodium acetate (pH 5.2) and 300 μl ethanol were added to 100 μl aqueous layer and stirred. The product was then centrifuged for 15 minutes at room temperature and 14,000 rpm and the sediment was recovered. Once the sediment was washed with 75% ethanol, it was dried under a vacuum for 2 minutes to obtain the sequencing sample. The sequencing sample was dissolved in formamide containing 4 μl 10 mM EDTA and denatured for 2 minutes at 90° C. This was then cooled in ice and submitted to sequencing. One of the 5 clones obtained had homology of the sequence with the probe used for PCR. In addition, DNA sequences extremely similar to the gene sequence of ribosomal protein L7/L12 gene of the other microorganisms, for example, Haemophilus influenzae, were discovered. The entire base sequence and the corresponding amino acid sequence of the structural gene moiety are as shown in SEQ ID NO.:21 and SEQ ID NO: 22 of the Sequence Table. This gene fragment clearly codes for Neisseria gonorrhoeae ribosomal protein L7/L12 protein.

Neisseria gonorrhoeae GST fused ribosome protein L7/L12 protein prepared by the same method as in Example 2 was obtained using the Neisseria gonorrhoeae GST fusion ribosomal protein L7/L12 protein expression vector constructed in this way. Furthermore, hybridoma strain AMGC1, which produces monoclonal antibody to ribosomal protein L7/L12 protein of Neisseria gonorrhoeae, was obtained in accordance with the method similar to the method of Example 3. Monoclonal antibody was produced and recovered in accordance with standard methods using the positive hybridoma cells of AMGC1 strain obtained as previously described.

Specifically, cells subcultivated in RPMI 1640 medium (containing 10% FCS) was diluted with a serum-free medium to about 2×10⁵ cells/ml, 3.3×10⁵ cells/ml, and 5×10⁵ cells/ml in 25 cm² culture flasks, and the total was made 5 ml. After cell were grown for 3 to 5 days in 7% CO₂ at 37° C., a flask which contains the least number of original cells was selected among flasks in which cells were grown. The same procedure was repeated until the cells diluted to 2×10⁵ cells/ml grow to 2×10⁶/ml in 3 to 4 days, thereby acclimatizing the cells with the serum-free medium. Next, cloning was performed in a 96-well plate for bacteria cultivation to select cells exhibiting fastest growth and a highest antibody titer. The selected cells were grown in a 24-well plate and diluted with a serum-free medium in a 25 cm² culture flask to a concentration of about 2×10⁵ cells/ml and the total volume was made 10 ml. After incubation for 3 to 4 days in 7% CO₂ at 37° C. to a concentration of 1×10⁶ cells/ml, the culture broth was transferred to a bottle for mass cultivation together with 100 ml of 1×10⁶ cells/ml cells which were grown in the same manner in a 75 cm² flask. 100 ml of a serum-free medium was added to the mixture, which was incubated at 37° C. for two days while stirring. 200 ml of the serum-free medium was added again and the mixture was incubated for a further two days. The culture broth was divided into four aliquot, the serum-free medium was added to each portion, followed by incubation for two days. After further addition of 400 ml of the serum-free medium, the culture broth was incubated for 6 days. The culture broth was collected and centrifuged at 10,000 rpm for 15 minutes to obtain a culture supernatant including the target antibody. After the addition of 0.1% sodium azide, the culture supernatant was stored at 4° C. 100 ml of the solution containing the antibody that was obtained was diluted to a 5-fold with PBS and adsorbed in a Protein A column (5 ml bed, Pharmacia) and rinsed with PBS at 3-times the bed volume. Then elution with citrate buffer at a pH of 3 was performed. The antibody fraction was recovered and monoclonal antibody that produced each hybridoma was obtained. The monoclonal antibody derived from the hybridoma was used in ELISA.

To evaluate the antibody, 96 well plates sensitized with ribosomal protein L7/L12 proteins of various microorganisms were used as an antigen. The monoclonal antibody prepared was reacted, followed by the reaction of horseradish peroxidase-labeled anti-mouse IgG (manufactured by MBL, Code 330) as a secondary antigen, and finally the antibody was detected using an enzyme reaction coloring reagent. In the ELISA reaction, solutions of recombinant ribosomal protein L7/L12 protein of Neisseria gonorrhoeae, Haemophilus influenzae, and Streptococcus pneumoniae dissolved in PBS containing 0.05% sodium azide diluted to 1 μg/ml were poured, 100 μl at a time, into separate 96-well plates and adsorbed overnight at 4° C. After removal of the supernatant, 200 μl 1% bovine serum albumin solution (in PBS) were added and reacted and blocked for 1 hour at room temperature. The supernatant was removed and the product was washed with a washing solution (0.02% Tween 20, PBS). Solutions of AMGC1 antibody, at concentrations of 0.1-1 μg/ml, in an amount of 100 μl each, were added and reacted for two hours at room temperature. The supernatant was removed and the product was further washed with a washing solution. Then, 100 μl of a solution of 5 μg/ml horse radish peroxidase-labeled anti-mouse IgG (manufactured by MBL, Code 330) was added and reacted for one hour at room temperature. The supernatant was removed and the product was washed with a washing solution. TMB (KPL) solution was added, 100 μl at a time, and reacted for 20 minutes at room temperature. After coloration, 100 μl 1 N sulfuric acid were added to stop the reaction. Absorbance at 450 nm was determined.

As a result, as shown in Table 6 it was confirmed that when the monoclonal antibody originating from hybridoma AMGC1 was used, this antibody can react with ribosomal protein L7/L12 proteins of all bacteria such as Neisseria gonorrhoeae, Haemophilus influenzae, and Streptococcus pneumoniae.

TABLE 6
Results of detection of AMGC1 antibody and
Ribosomal Protein L7/L12 of microorganisms

| | |
|---|---|
| Neisseria gonorrhoeae | + |
| Haemopbilus influenzae | + |
| Streptococcus pneumoniae | + |

(+: Positive)

The AMGC1 antibody obtained here is very useful as an antibody used for the detection of microorganisms in the so-called sandwich assay by optical immunoassay and ELISA method, in combination with an anti-ribosomal protein L7/L12 antibody which is specific to each microorganism.

Chlamydia pneumoniae

An object of the present invention is also to provide a method for specifically and rapidly detecting a microorganism that belongs to Chlamydia pneumoniae, a detection antibody used for the detection and a reagent kit for the detection. Furthermore, another object of the present invention is to provide a method for manufacturing the detection antibody used for the detection.

The inventors have identified a useful protein antigen that is conserved same function in all microorganisms. Generally, the structural change of said protein is expected to be very low. Surprisingly, it has been found that the antibody to the protein is specific to species or genus of microorganism, has a protean property enable to use for discrimination specific to species or genus of microorganism, and object microorganisms may be detected all serotypes thereof.

The inventors focused on intracellular molecules that are present as molecules having same function in all microorganism cells and somewhat differ between microorganisms in terms of it's amino acid sequence, particularly Ribosomal Protein L7/L12 that is one kind of ribosomal protein. Ribosomal Protein L7/L12 is a protein with a molecular weight of approximately 13 kilo Daltons and is known to exist as an essential ribosomal protein in protein synthesis. Progress has been made in understanding

the complete amino acid sequence of Ribosomal Protein L7/L12 in several microorganisms including Chlamydia pneumoniae.

The inventors focused on the fact that even though there are similarities between different microorganisms in terms of said molecule, this molecule also has a structural segment that is unique to each microorganism and discovered that, it is possible to detect various microorganisms with species specificity and to detect all serotypes within the same species by using antibody to said protein.

The inventors completed the present invention upon discovering that antibody specific to the protein of Chlamydia pneumoniae can be obtained and species-specific detection of Chlamydia pneumoniae is possible using said antibody.

In accordance with the present invention a monoclonal antibody specific to Ribosomal Protein L7/L12 of Chlamydia pneumoniae has been discovered and developed. The antibody is novel and different from any antibody previously known and has property of reacting specifically to the said protein.

SEQ ID NO:23 and SEQ ID NO:24 in the Sequence List are the DNA sequence (NCBI database accession #NC#000922) of the Ribosomal Protein L7/L12 gene of Chlamydia pneumoniae and the corresponding amino acid sequence (NCBI database accession #AE001593.1, NCBI data base). The left terminal and right terminal of the amino acid sequences entered in the Sequence List are amino group (referred to below as the, N terminal) and carboxyl group terminals (referred to below as the C terminal), respectively, and the left terminal and right terminal of the base sequence is the 5′ terminal and the 3′ terminal, respectively. Amino acid in the sequence of closest match test is expressed by one letter notation of amino acid. The notation “+” in closest match test indicates that it is different amino acid but amino acid with similar properties such as hydrophobic. The notation “ ” (blank) indicates that it is entirely different amino acid including properties thereof. Moreover, the series of bio-molecular experiments of gene preparation mentioned in this text can be performed by methods entered in standard experimental manuals. “Molecular cloning: A laboratory manual”, Cold Spring Harbor Laboratory Press, Sambrook, J. et al. (1989), is given as an example of the well-known standard experimental manual.

TABLE 7
Closest Match Test:

| | | | |
|---|---|---|---|
| Ct: | 1 | MTTESLETLVEQLSGLTVLELSQLKKLLEEKWDVTAAAPVVAVAGAAAAGDAPASAEPTE | 60 |
| | | +TTESLETLVE+LS LTVLELSQLKKLLEEKWDVTA+APVVAVA A G+AP +AEPTE | |
| Cp: | 1 | VTTESLETLVEKLSNLTVLELSQLKKLLEEKWDVTASAPVVAVA−AGGGGEAPVAAEPTE | 59 |
| Ct: | 61 | FAVILEDVPSDKKIGVLKVVREVTGLALKEAKEMTEGLPKTVKEKTSKSDAEDTVKKLQE | 120 |
| | | FAV LEDVP+DKKIGVLKVVREVTGLALKEAKEMTEGLPKTVKEKTSKSDAEDTVKKLQ+ | |
| Cp: | 60 | FAVTLEDVPADKKIGVLKVVREVTGLALKEAKEMTEGLPKTVKEKTSKSDAEDTVKKLQD | 119 |
| Ct: | 121 | AGAKAVAKGL 130 | |
| | | AGAKA KGL | |
| Cp: | 120 | AGAKASFKGL 129 | |

Ct = Chlamydia trachomatis
Cp = Chlamydia pneumoniae

In the present invention, the term “microorganism” means Chlamydia pneumoniae, specifically, indicates microorganism having a pathogenic property in respiratory organ and high significance in diagnosis as a causative pathogen of Chlamydia infections.

In the present invention, the term “antibody specifically reacting with microorganism” indicates an antibody that can specifically react with species or genus of microorganism, and antibody specifically reacting with species is especially useful in diagnosis of bacterial infections.

The term “antibody” in the present invention means a polyclonal antibody or monoclonal antibody that can be made using the entire length or only a partial peptide of said Ribosomal Protein L7/L12. Although there are no special restrictions to the peptide length for making the antibody, in the case the antibody to Ribosomal Protein L7/L12, the segment may be of the length characterizing the Ribosomal Protein L7/L12, and a peptide of 5 amino acids or longer, particularly 8 amino acids or longer, is preferred.

Antiserum containing antibody (polyclonal antibody) that identifies Ribosomal Protein L7/L12 can be obtained by inoculating laboratory animals with adjuvant and a peptide or the full length protein, as is or, when necessary, after being cross-linked with a carrier protein such as KLH (keyhole-limpet hemocyanin) and BSA (bovine serum albumin) and recovering the serum. Moreover, the antibody can be used after it has been purified from the antiserum. The laboratory animals that are inoculated include sheep, horses, goats, rabbits, mice, rats, etc., and sheep, rabbits, etc., are particularly preferred for preparation of monoclonal antibody. Moreover, monoclonal antibody can also be obtained by conventional methods of making hybridoma cells, but mice are preferred in this case.

The entire length of said protein, or its amino acid sequence of 5 or more, preferably 8 or more, residues that has been fused with glutathione S-transferase (GST), etc., can be purified and used as antigen, or it can be used as antigen without being purified. The antibody can also be produced from the genetic recombination antibody expressed in cultured cell using immunoglobulin genes that have been separated by a variety of methods in published documents (“Antibodies: A Laboratory manual,” E. Harlow et al., Cold Spring Harbor Laboratory), cloning methods, etc.

Antibody to Ribosomal Protein L7/L12 that can be employed as the marker antigen of the present invention can be obtained by the following methods and other similar methods as well, though not to be limited within these methods:

a) The desired antibody can be acquired by synthesizing a peptide fragment; in the case microorganism has a known Ribosomal Protein L7/L12 genetic sequence and amino acid sequence, using the region least similar to the amino acid sequence of said protein of another bacteria and making polyclonal antibody, or monoclonal antibody, using this peptide fragment as the immune source.

Moreover, it is possible to acquire the entire sequence of said gene by using a conventional genetic procedure, such as gene amplification by PCR using the DNA sequence at both terminals of said known genetic sequence as the probe, or hybridization using the sequence of a homologous segment as the template probe.

Then, a fused gene with another protein gene is constructed and said fused gene is inserted into the host by conventional gene insertion methods using Escherichia coli, etc., as the host and expressed in large quantities. The desired protein antigen can then be acquired by purifying the expressed protein by affinity column methods with antibody to the protein that was used as the fusion protein. In such a case, even if antibody to the amino acid segment retained within the microorganism is acquired, it does not coincide with the purpose of the present invention because the full length of Ribosomal Protein L7/L12 becomes the antigen. Consequently, hybridoma that produces monoclonal antibody to the antigen that has been obtained by this method is acquired by conventional methods and the desired antibody can be obtained by selecting a clone, which produces antibody that will react only with the desired microorganism.

b) For microorganism that the amino acid sequence of the Ribosomal Protein L7/L12 is unknown, as the amino acid sequence of the Ribosomal Protein L7/L12 has 50-60% of homology between microorganism, the protein gene can easily obtained by using a conventional genetic procedure, such as gene amplification of specific sequence moiety by PCR based on the sequence of a homologous segment of the amino acid sequence, or hybridization using the sequence of a homologous segment as the template probe.

Then, a fused gene with another protein gene is constructed and said fused gene is inserted into the host such as Escherichia coli, and the like by conventional gene insertion methods, and expressed in large quantities. The desired protein antigen can then be acquired by purifying the expressed protein by affinity column methods with antibody to the protein that was used as the fusion protein. In such a case, even if antibody to the amino acid segment retained within the microorganism is acquired, it does not coincide with the purpose of the present invention because the full length of Ribosomal Protein L7/L12 becomes the antigen. Consequently, hybridoma that produces monoclonal antibody to the antigen that has been obtained by this method is acquired by conventional methods and the desired antibody can be obtained by selecting a clone, which produces antibody that will react only with the desired microorganism.

c) Alternatively, as another method that is suitable for the case where the amino acid sequence of the Ribosomal Protein L7/L12 is unknown, a peptide of 5 to 30 amino acids corresponding to the common sequence segment retained in the microorganism is synthesized from the known amino acid sequence of the Ribosomal Protein L7/L12, and polyclonal antibody or monoclonal antibody to this peptide sequence is made by conventional methods. Then Ribosomal Protein L7/L12 highly purified can be obtained by purifying the disrupted liquid of bacterial cells through affinity column chromatography using said antibody.

If purity of the protein is insufficient, it can be purified by conventional methods, such as ion exchange chromatography, hydrophobic chromatography, gel filtration, etc., after which the eluted fraction of Ribosomal Protein L7/L12 is identified by method such as western blotting using antibody that was made, and purified protein can be obtained. The desired antibody can be obtained by acquiring hybridoma by conventional methods using the purified Ribosomal Protein L7/L12 antigen that has been obtained, and selecting hybridoma reactable specifically with the desired microorganism.

The antibody of the present invention specific to various microorganisms that has been obtained by the methods in a), b) and c) can be used in various diagnostic reagents and kits specific to microorganisms, and can be used in a variety of immunoassay methods. For example, this antibody can be used in aggregation reactions, that is one of known measuring method and where antibody is adsorbed on polystyrene latex particles, ELISA, which is a conventional technology performed in a microtiter plate, conventional immunochromatography methods, and sandwich assay, whereby said antibody labeled with colored particles or particles having coloring capability, or with enzyme or fluorescence substance, and magnetic micro-particles coated with capture antibody, etc., are used, etc.

The term-“microorganism diagnosis methods using antibody” means diagnostics methods using any known conventional immunoassay, such as aggregation whereby said antibody is adsorbed on polystyrene latex particles, ELISA, which is a conventional method performed in a microtiter plate, conventional immunochromatography methods, or sandwich assay, whereby said antibody labeled with colored particles or particles having coloring capability, or with enzyme or fluorescence substances, and magnetic micro-particles coated with capture antibody, etc., are used.

Moreover, the optical immunoassay (OIA) technology described in Japanese (via International) Patent Application Laid-open No. 07(1995)-509565, in which microorganisms are detected by the principle of an optical interference induced by an antibody reaction on the optical thin film which is formed by silicone, silicon nitride or the like, is a useful as a high sensible diagnostic method, especially as a diagnostic method of microorganisms using an antibody.

Moreover, as a method for extracting intracellular marker antigen from the desired microorganism in aforementioned diagnostic method, reagent treatment using an extraction reagent(s) comprising various surfactants, such as typically Triton X-100 and Tween-20, enzyme treatment using an appropriate enzyme, such as protease, etc., and physical treatment using known cell structure crushing methods, typically cell-crushing of microorganism, can be used. It is preferred that the most suitable conditions for extracting with reagent are set to each kind of microorganism using a proper combination of surfactants, etc.

Moreover, in the present invention the term “reagent kit for diagnosis of microorganisms using antibody” means a diagnostic kit that uses the above-mentioned diagnostic method.

The amino acid and DNA sequence of Ribosomal Protein L7/L12 of Chlamydia pneumoniae are shown as SEQ ID NO: 23 and SEQ ID NO: 24 in Sequence List, respectively. Consequently, in the case of this microorganism, it is possible to compare the amino acid sequence of Ribosomal Protein L7/L12 with the same protein of closely related microorganisms. Which is shown in Sequence List under the heading “Closest Match”. Synthesizing a peptide with the segment of low homology and making polyclonal or monoclonal antibody to that could short cut the selection of those having specificity to the microorganisms.

Especially in the case of a polyclonal antibody, it is preferred that IgG fraction be obtained by purification of the antiserum of immunized laboratory animals with a protein A column, etc., and affinity purification be performed with the synthetic peptide used in immunization of the laboratory animals.

Moreover, PCR primers are formed based on the sequences of N-terminal and C-terminal from the DNA sequence of Ribosomal Protein L7/L12 of the microorganism. Utilizing homology of the PCR primers, DNA fragments are amplified by the PCR method using genomic DNA and extracted, the fragments of Chlamydia pneumoniae can be thus acquired according to a conventional method. The entire length of the gene for Ribosomal Protein L7/L12 of Chlamydia pneumoniae can be acquired through the analysis of the DNA sequence information of these fragments.

The Ribosomal Protein L7/L12 gene of Chlamydia pneumoniae thus acquired forms a fusion protein gene with, for example, GST, etc., and an expression vector is built using an appropriate expression plasmid, Escherichia coli is transformed and a large quantity of said protein can be expressed. A suitable amount of the transformed Escherichia coli is cultivated and disrupted bacterial fluid is subjected to purification by an affinity column using GST to obtain the GST fusion Ribosomal Protein L7/L12 of Chlamydia pneumoniae.

It is also possible to acquire the target specific monoclonal antibody by establishing a multiple clone of hybridomas using said protein as is or GST moiety deleted protein as an antigen protein, and selecting the antibody which exhibits a specific response to Chlamydia pneumoniae bacteria, a homogenized fluid of the bacteria, or Ribosomal Protein L7/L12 of Chlamydia pneumoniae.

Antibody made based on the present invention can be used in all known types of immunoassay, such as known aggregation reaction whereby said antibody is adsorbed on polystyrene latex particles, ELISA, which is a conventional technology performed in a microtiter plate, conventional immunochromatography, and sandwich assay, whereby said antibody labeled with colored particles or particles that have coloring capability, or enzymes or fluorescence substances, and magnetic particles coated with capture antibody are used, etc.

Moreover, antibody that is made based on the present invention can simultaneously function as a so-called capture antibody that captures said antigen protein in solid or liquid phase and as a detecting antibody that is a so-called enzyme-labeled antibody by modificating an enzyme, such as peroxidase and alkali phosphatase, etc., by conventional methods in any of these immunoassay procedure.

The following examples are given to explain specifically the present invention; the present invention the principle of is not being restricted to these examples.

### Example 11

Cloning of Ribosomal Protein L7/L12 Genes from Chlamydia pneumoniae

Chlamydia pneumoniae (ATCC VR-1310; distributed and purchased from ATCC) was cultured on a monolayer of HL cell line. Detailed procedure for culturing Chlamydia pneumoniae is described by Kuo et al. and the like (Cles and Stamm 1990; Kuo and Grayston 1990; Yoshizawa, Dairiki et al. 1992). The microorganism was cultivated for 5 days in a CO₂ incubator under conditions of 37° C. and 5% C02. Infected cells were collected by centrifugation and suspended in a TE buffer (Wako Pure Chemical Industries, Ltd.) to a final concentration of approximately 5×10⁷ cells/ml. Approximately 1.5 ml of this suspension was transferred to a microcentrifuge tube and centrifuged for 2 minutes at 10,000 rpm. The supernatant was discarded. The sediment was resuspended in 567 μl of TE buffer. Then 30 μl of 10% SDS and 3 μl of 20 mg/ml Proteinase K solution were added and thoroughly mixed, and the suspension was incubated for one hour at 37° C. The suspension was incubated for another one hour at 56° C. After adding 80 μl of 10% acetyl trimethyl ammonium bromide/0.7 M NaCl solution, it was incubated for 10 minutes at 65° C. 700 μl of chloroform-isoamyl alcohol solution at a volume ratio of 24:1 was added and stirred well.

The solution was centrifuged for 5 minutes at 4° C. and 12,000 rpm using a microcentrifugation device and the aqueous fraction was transferred to a new microcentrifuge tube. Isopropanol was added to the fraction at 0.6-times its volume and the tube was vigorously shaken to form sediment of the DNA. The white DNA sediment was scooped with a glass rod and transferred to a different microcentrifugation tube containing 1 ml of 70% ethanol (cooled to −20° C.). The tube was centrifuged for 5 minutes at 10,000 rpm and the supernatant was gently removed. Then another 1 ml of 70% ethanol was added and the mixture was centrifuged for 5 more minutes.

Once the supernatant had been removed, the sediment was dissolved in 100 μl of TE buffer to obtain the DNA solution. The concentration of the genomic DNA solution was determined quantitatively in accordance with ES, Spectrophotometric Determination of the Amount of DNA or RNA in “Molecular cloning: A laboratory manual”, Cold Spring Harbor Laboratory Press, Sambrook, J. et al. (1989).

PCR (polymerase chain reaction) was performed using long of this genomic DNA. Taq polymerase (Takara Co., ltd., code R001A) was used for PCR. Five pl of buffer attached the enzyme, 4 μl of a dNTP mixture attached the enzyme, and 200 pmol of each synthetic oligonucleotide (shown in SEQ ID NO:25 and 26 of the Sequence List) were added to the enzyme. Purified water was added to bring the final volume to 50 μl.

This mixture was cycled 5 times with a TaKaRa PCR Thermal Cycler 480 for 1 minute at 95° C., 2 minutes at 5° C., and 3 minutes at 72° C. and was then cycled 25 times for 1 minute at 95° C., 2 minutes at 60° C., and 3 minutes at 72° C. Electrophoresis was performed in 1.5% agarose gel using a part of this PCR product. This product was then stained with ethidium bromide (Nippon Gene Co., Ltd.) and observed under ultraviolet ray to confirm amplification of approximately 400 bp DNA. After fragmentation treatment with restriction endonucleases BamHI and Xhol, electrophoresis was performed in 1.5% agarose gel and staining with ethidium bromide was carried out. An approximately 400 bp band was cut out from the gel. This band was purified with Suprecol (Takara Co., Ltd.) and then inserted into pGEX-6P-1 (Pharmacia), which is a common vector. This vector can function as an expression vector for the desired molecule, which can express fused protein with GST protein, by insertion of the desired gene fragment into the appropriate restriction endonuclease site.

Specifically, vector pGEX-6P-l and the previous DNA were mixed together at a molar ratio of 1:3 and DNA was inserted into the vector with T4 DNA ligase (Invitrogen Co.). Vector pGEX-6P-l into which DNA had been inserted was genetically introduced to Escherichia coli one-shot competent cells and then inoculated in a plate of LB L-broth agar (Takara Co., ltd.) which was semi-sold culture plate containing 50 μg/ml ampicillin (Sigma). The plate was then incubated at 37° C. for 12 hours and the grown colonies were selected at random and inoculated into L-Broth liquid culture medium containing the same concentration of ampicillin. Shake cultivation was performed at 37° C. for 8 hours and the bacteria were recovered and the plasmid was separated using Wizard Miniprep in accordance with the attached description. The plasmid was cleaved with restriction endonuclease BamHI/XhoI. Insertion of said PCR product was confirmed by cutting out approximately 370 bp DNA. The base sequence of the DNA that had been inserted was determined using said clone.

Determination of the base sequence of the inserted DNA fragment was performed using the Fluorescence Sequencer of Applied Biosystems.

The sequence sample was prepared using PRISM, Ready Reaction Dye Terminator Cycle Sequencing Kit (Applied Biosystems). First, 9.5 μl of reaction stock solution, 4.0 μl of 0.8 pmol/pl T7 promoter primer (Gibco BRL) and 6.5 μl of 0.16 μg/μl template DNA were added to a microtube with a capacity of 0.5 ml and mixed. After covering the mixture with a double layer of 100 μl mineral oil, PCR amplification was performed for 25 cycles, where one cycle consisted of 30 seconds at 96° C., 15 seconds at 55° C., and 4 minutes at 60° C. The product was then kept at 4° C. for 5 minutes. After the reaction was completed, 80 μl sterilized pure water was added and stirred. The product was centrifuged and the aqueous layer was extracted 3 times with phenol-chloroform mixed solution. Ten microliters 3M-sodium acetate (pH 5.2) and 300 μl ethanol were added to 100 μl aqueous layers and stirred. The product was then centrifuged for 15 minutes at room temperature and 14,000 rpm and the sediment was recovered. Once the sediment was washed with 75% ethanol, it was dried under a vacuum for 2 minutes to obtain the sequencing sample. The sequencing sample was dissolved in formamide containing 4 pl of 10 μM EDTA and denatured for 2 minutes at 90° C. This was then cooled in ice and submitted to sequencing. Two out of the 5 randomly selected clones had homology of the sequence with the probe used for PCR. In addition, a DNA sequence was evidently identical to the gene sequence of Ribosomal Protein L7/L12. The entire base sequence and the corresponding amino acid sequence of the structural gene moiety are as shown in SEQ ID NO:25 and SEQ ID NO: 26 of the Sequence List. This gene fragment clearly codes for Chlamydia pneumoniae Ribosomal Protein L7/L12.

### Example 12

Mass Expression in Escherichia coil and Purification of Ribosomal Protein L7/L12 from Chlamydia pneumoniae

Escherichia coli into which expression vector had been inserted was cultivated overnight in 50 ml of LB medium at 37° C. Then 500 ml of 2-times concentrated YT medium was heated at 37° C. for 1 hour. Fifty milliliters of the Escherichia coli solution that had been cultivated overnight were introduced to 500 ml of the aforementioned medium. One hour later, 550 μl of 100 mM isopropyl-β-D(−)-thiogalactopyranoside (IPTG) were introduced and cultivated for 4 hours. The product was then recovered and introduced to 250 ml centrifugation tubes and centrifuged for 10 minutes at 7,000 rpm. The supernatant was discarded and dissolved in 25 ml each of Lysis buffer containing 25% sucrose in 50 mM Tris buffer, pH 7.4. Furthermore, 1.25 ml of 10% NP-40 and 125 μl of 1M MgCl₂ were added and the mixture was transferred to a plastic tube. Ultrasonication was performed 1 minute×5 times while ice cold. The product was centrifuged for 15 minutes at 12,000 rpm and the supernatant was recovered.

Next, the aforementioned supernatant was adsorbed on a glutathione agarose column conditioned with PBS. Then the column was washed with twice bed volume of washing solution containing 4.2 mM MgCl₂ and 1 mM dithiothreitol (DTT) in 20 mM Tris buffer, pH 7.4. Elution was performed in 50 mM Tris buffer, pH 9.6, containing 5 mM glutathione. The protein content in the elution fraction was determined by the pigment bonding method (Bradford method; BioRad Co.) and the main fraction was acquired.

Purity of the purified GST fusion Ribosomal Protein L7/L12 that was obtained was confirmed by electrophoresis to be approximately 75%, showing the purity satisfactory for an immunogen.

### Example 13

Preparation of Monoclonal Antibody to Ribosomal Protein L7/L12 of Chlamydia pneumoniae

First, regarding to the immunization of mice, 100 μg of the GST fusion of Ribosomal Protein L7/L12 antigen of Chlamydia pneumoniae were dissolved in 200 μl of PBS and then 200 μl of Freund's complete adjuvant were added and mixed and emulsification was performed. Two hundred microliters of the emulsion were injected intraperitoneally to immunize mice. Then the same emulsion antigen was intraperitoneally injected after 2 weeks, after 4 weeks, and after 6 weeks. Two-fold the concentration of antigen emulsion was injected intraperitoneally after 10 weeks and after 14 weeks. The spleen was excised out 3 days after the final immunization and submitted to cell fusion.

After thoroughly mixing 2×10⁷ myeloma cells per 10⁸ spleen cells, which had been recovered aseptically from mice, in a glass tube, the mixture was centrifuged for 5 minutes at 1,500 rpm and the supernatant was discarded. The cells were then thoroughly mixed.

The myeloma cells used for cell fusion were obtained by cultivation of cell strain NS-1 with an RPMI 1640 culture medium containing 10% bovine fetal serum, cultivating this product using an RPMI 1640 medium containing 0.13 mM azaguanine, 0.5 μg/ml MC-210, and 10% bovine fetal serum for 1 weeks from 2 weeks before the cell fusion, and then further cultivating the cell strain for 1 week with an RPMI 1640 medium containing 10% bovine fetal serum.

Fifty ml of RPMI 1640 culture medium that had been kept at 37° C. were added to the mixed cell sample and centrifuged at 1,500 rpm. After removing the supernatant, 1 ml of 50% polyethylene glycol that had been kept at 37° C. was added and stirred for 1 minute. Ten ml of RPMI 1640 medium kept at 37° were added and the mixed solution was vigorously mixed for approximately 5 minutes by sucking and discharging the mixed solution with a sterile pipette.

After centrifugation for 5 minutes at 1,000 rpm and removal of the supernatant, 30 ml of HAT culture medium were added to bring the cell concentration to 5×10⁶ cells/ml. This mixture was stirred till uniform and then poured, 0.1 ml per each well, into a 96-well culture plate and cultivated at 37° C. and under condition of 7% carbon dioxide gas. HAT culture was added, 0.1 ml at a time, on the first day, at after 1 week and after 2 week, respectively. Then the cells that had produced the desired antibody were screened by ELISA.

GST fusion Ribosomal Protein L7/L12 and GST protein were dissolved in PBS containing 0.05% sodium azide and diluted to 10 μg/ml. The diluted solutions were separately poured, 100 μl per each well, into 96-well plates and adsorbed overnight at 4° C.

After removing the supernatant, 200 μl of 1% bovine serum albumin solution in PBS were added and the mixture was reacted and blocked for 1 hour at room temperature. After removing the supernatant, the product was washed with washing solution (0.02% Tween 20, PBS). One hundred microliters of culture solution of fused cells were added to this and reacted for 2 hours at room temperature. The supernatant was removed and the sediment was washed with washing solution. Next, 100 μl of 50 ng/ml peroxidase-labeled goat anti-mouse IgG antibody solution were added and the mixture was reacted for 1 hour at room temperature. The supernatant was removed and the product was washed again with washing solution. Then TMB solution (KPL Co., Ltd.) was added, 100 μl each, and the mixture was reacted for 20 minutes at room temperature. After coloration, 100 μl of 1N sulfuric acid were added to stop the reaction and absorbance at 450 nm was determined.

As a result, positive cells that reacted only with GST fusion Ribosomal Protein L7/L12 but did not react with GST protein were detected, thus it could be concluded that antibody to Ribosomal Protein L7/L12 is produced.

Therefore, the cells in the positive wells were recovered and cultivated with HAT medium in a 24-well plastic plate.

The fused medium that had been cultivated was diluted with HT medium to a cell number of approximately 20 cells/ml and then 50 μl of the diluted medium was mixed with 10⁶ six-week-old mouse thymus cells suspended in HT culture medium in a 96-well culture plate. The culture was then cultivated for 2 weeks at 37° C. and under conditions of 7% carbon dioxide gas.

Antibody activity in the culture supernatant was similarly determined by the aforementioned ELISA method and the cells that showed positive reaction with Ribosomal Protein L7/L12 were recovered. Furthermore, the same dilution detection and cloning procedure was repeated to obtain 5 clones in total as hybridoma CPRB-1--5.

### Example 14

Selection of Monoclonal Antibody That Detects Ribosomal Protein L7/L12 of Chlamydia pneumoniae.

Monoclonal antibody was produced and recovered in accordance with conventional methods using the positive hybridoma cells obtained as previously described.

Specifically, 5×10⁶ cells in PBS that had been subcultured using RPMI 1640 culture medium containing 10% FCS were intraperitoneally injected into Balb/C mice that had been intraperitoneally injected with 0.5 ml Pristane before 2 weeks in advance. Ascites was recovered 3 weeks later and the centrifugation supernatant was obtained.

The obtained solution containing antibody was adsorbed in a Protein A column (5 ml, Pharmacia) and rinsed with PBS at 3-times volume. Then elution with citrate buffer, pH 3, was performed. The antibody fraction was recovered and the monoclonal antibody that produced by each hybridoma was obtained. The monoclonal antibody derived from these 5 strains of hybridoma was evaluated by ELISA method.

The sandwich assay method was used to evaluate the monoclonal antibody. The monoclonal antibody that was prepared was used as antibody for detection by being chemically bound to peroxidase.

That is, enzyme labeling was performed using horseradish peroxidase (Sigma Grade VI) in accordance with the method described in “Analytical Biochemistry 132 (1983), 68-73” using the reagent S-acetylthioacetic acid N-hydroxysuccinimide for binding. In the ELISA reaction, a solution of a commercially available anti-Chlamydia pneumoniae polyclonal antibody (rabbit) diluted to a concentration of 10 μg/ml was separately poured, 100 μl per each well, into a 96-well plate and adsorbed overnight at 4° C.

After removing the supernatant, 200 μl of 1% bovine serum albumin solution in PBS were added and the mixture was reacted and blocked for 1 hour at room temperature. The supernatant was removed and the product was washed with washing solution containing 0.02% Tween 20, in PBS. One hundred microliters of antigen solution, which had been obtained by adding Triton X-100 to culture solutions of each species of microorganism to a concentration of 0.3% and then extracting the solution for 5 minutes at room temperature, were added to this and the mixture was reacted for 2 hours at room temperature. The supernatant was removed and the product was washed again with washing solution. Then 100 μl of 5 μg/ml peroxidase-labeled anti-Ribosomal Protein L7/L12 antibody solution were added and the mixture was reacted for 1 hour at room temperature. The supernatant was removed and the product was washed with washing solution. TMB (KPL) solution was added, 100 μl each, and the mixture was reacted for 20 minutes at room temperature. After coloration, 100 μl of 1N sulfuric acid were added to stop the reaction. Absorbance at 450 nm was determined.

It is evident that when monoclonal antibody derived from hybridoma CPRB-l was used as the enzyme-labeled antibody, all strains of Chlamydia pneumoniae tested were detected at a sensitivity of 10⁶ cells/ml, while reactivity of other microorganisms, such as Haemophilus influenzae, Klebsiella pneumoniae, Mycoplasma pneumoniae and Neisseria meningitides could not be detected, even at high concentrations of 10⁸ cells/ml and therefore, antibody with specific reactivity to Chlamydia pneumoniae can be obtained by using monoclonal antibody to Ribosomal Protein L7/L12. The antibody was named as AMCP-1. Table 8 shows only those results with AMCP-1. Results with other antibodies that cross-reacted with other microorganisms are not mentioned here.

TABLE 8

| | Result of Detection |
|---|---|
| | (10⁶ cells/ml) |
| C. pneumoniae | + |
| | Results of Detection |
| | (10⁸ cells/ml) |
| N. meningitides | − |
| N. lactamica | − |
| N. mucosa | − |
| N. sicca | − |
| M. pneumoniae | − |
| H. influenzae | − |
| B. catarrharis | − |
| N. gonorrhoeae | − |
| E. coli | − |
| K. pneumoniae | − |

+: Positive, −: Negative)

### Example 15

Acquisition of a Polyclonal Antibody, Which Specifically Reacts with Ribosomal Protein L7/L12 of Chlamydia pneumoniae Using a Ribosomal Protein L7/L12-Immobilized Affinity Column

Ribosomal Protein L7/L12 of Chlamydia pneumoniae which was acquired by the method described in Examples 1, or the supernatant of Chlamydia pneumoniae bacteria treated with Triton X-100 was used as an antigen. About 1.2 ml of a physiological saline solution containing 100 μg of antigen was emulsified with the addition of 1.5 ml of Freund's adjuvant. The emulsion was subcutaneously injected into SPF Japanese White Rabbit to immunize the rabbit. The rabbit was immunized 5 to 6 times once every two weeks, and the antibody titer was confirmed.

The antibody titer was confirmed by the ELISA method. Ribosomal Protein L7/L12 of Chlamydia pneumoniae was dissolved in PBS containing 0.05% sodium azide and diluted to concentration of 10 μg/ml. The diluted solution was poured, 100 μl per each well, into 96-well plates and adsorbed overnight at 4° C. After removing the supernatant, 200 μl of 1% bovine serum albumin solution in PBS were added and the mixture was reacted and blocked for 1 hour at room temperature. The supernatant was removed and the product was washed with a washing solution containing 0.02% Tween 20, in PBS. One hundred μl of a solution obtained by diluting normal rabbit serum and immunized rabbit antiserum was added and the mixture was reacted for two hours at room temperature. The supernatant was removed and the product was washed again with a washing solution. Then, 100 μl of 50 ng/ml peroxidase-labeled goat anti-rabbit IgG antibody solution was added and the mixture was reacted for one hour at room temperature. The supernatant was removed and the product was washed with a washing solution. OPD solution (Sigma Co.) was added, 100 μl each, and each mixture was reacted for 20 minutes at room temperature. After coloration, 100 μl of 1N sulfuric acid was added to stop the reaction. Absorbance at 492 nm was determined.

After confirming that the antibody titer had increased, a large quantity of blood was collected. Blood was collected in a glass centrifuge tube from the ear artery, allowed to stand for one hour at 37° C., and then overnight at 4° C. The blood was centrifuged at 3,000 rpm for 5 minutes and the supernatant was recovered. The resulting anti-serum was stored at 4° C.

An affinity column immobilized Ribosomal Protein L7/L12 of Chlamydia pneumoniae was prepared. HiTrap NHS-activated column (1 ml, manufactured by Pharmacia was used. Immediately after replacing the column with 1 mM HCl, a solution of Ribosomal Protein L7/L12 in PBS (1 mg/ml) was charged. The column was allowed to stand for 30 minutes and a blocking reagent was charged, followed by equilibration with PBS.

Using the affinity column immobilized Ribosomal Protein L7/L12 of Chlamydia pneumoniae, the polyclonal antibody in the resulting anti-serum obtained as an antigen from the supernatant of Triton X-100 treated bacteria of Chlamydia pneumoniae was purified. This antiserum was diluted with PBS to 5 times of its volume, passed through a 0.45 μm filter, and then adsorbed in the column immobilized Ribosomal Protein L7/L12 of Chlamydia pneumoniae at a flow rate of 0.5 ml/min. After elution from the column with 0.1 M glycine, pH 2.1, the eluted fraction was immediately neutralized with 1 M Tris buffer, pH 9.0, the target antibody in eluted fraction was then recovered by the ELISA method similar to the antibody-titer measuring method.

The polyclonal antibody obtained in this manner was evaluated by the OIA method as described in Japanese (via International) Patent Application Laid-open No. 07(1995)-509565.

The purified antibody was used as a capture antibody for the OIA method. Moreover, peroxidase-labeled AMCP-1 monoclonal antibody described in Example-4 was used as the detect antibody. That is, enzyme labeling was performed in accordance with the method described in “Analytical Biochemistry 132 (1983), 68-73” using horseradish peroxidase (Sigma Grade VI) and the reagent S-acetylthioacetic acid N-hydrixtsuccinimide for binding.

In the OIA reaction, the purified polyclonal antibody in PBS containing 0.05% sodium azide was diluted with 0.1 M HEPES buffer, pH 8.0, to a concentration of 10 μg/ml and the diluted was added onto a silicon wafer, 50 μl at a time, to react for 30 minutes at room temperature, followed by washing with distilled water and coating with a coating solution including sucrose and alkali treated casein.

Fifteen μl of antigen solution, which had been obtained by adding Triton X-100 to culture solutions of each bacterium to a concentration of 0.5% and then extracting the solution for 5 minutes at room temperature, was added onto the above silicon wafer and reacted for 10 minutes at room temperature. Then, 15 μl of 20 μg/ml peroxidase-labeled monoclonal antibody was added and reacted for 10 minutes. After washing with distilled water, TMB solution (KPL) was added, 15 μl at a time, and reacted for 5 minutes at room temperature. The product was washed with distilled water and observed the blue generated from the enzyme reaction.

As a result, as shown in Table 9 it is clear that when the purified polyclonal antibody APCP-1 is used as the capture antibody, Chlamydia pneumoniae can be detected in a sensitivity of 10⁸ cells/ml, while reactivity of other microorganisms cannot be detected. Thus, an affinity column immobilized with the Ribosomal Protein L7/L12 of Chlamydia pneumoniae confirmed the capturing an antibody having specific reactivity to Chlamydia pneumoniae.

TABLE 9

Results of Detection
(10⁸ cells/ml)

| | | |
|---|---|---|
| C. pneumoniae | | + |
| H. influenzae | ATCC10211 | − |
| E. coli | ATCC25922 | − |
| E. faecalis | ATCC19433 | − |
| K. pneumoniae | ATCC13883 | − |
| N. gonorrhoeae | IID821 | − |
| N. lactamica | ATCC23970 | − |
| N. meningitidis | ATCC13090 | − |
| P. aeruginosa | ATCC27853 | − |
| Group B | ATCC12386 | − |
| Streptococcus | ATCC25923 | − |
| S. aureus | ATCC27336 | − |
| S. pneumoniae | ATCC19615 | − |
| S. pyogenes | | |

(+: Positive, −: Negative)

Mycoplasma pneumoniae

A further object of the present invention is to provide a method for manufacturing the detection antibody using for the detection.

The inventors have identified a useful protein antigen that is conserved same function in all bacteria. Generally, the structural change of said protein is expected to be very low. Surprisingly, it has been found that the antibody to the protein is specific to bacterial species or genus, has a protean property enable to use for discrimination specific to bacterial species or genus, and object microorganisms may be detected all serotypes thereof. In more detail, said proteins are useful proteins exhibiting species-specific property that the structures, through the change of the amino acid sequence, are completely same in same species, but structural changes are accompanied in the case species are different.

The inventors focused on intracellular molecules that are present as molecules having same function in all microorganism cells and somewhat differ between microorganisms in terms of it's amino acid sequence, particularly Ribosomal Protein L7/L12 that is one kind of ribosomal protein. Ribosomal Protein L7/L12 is a protein with a molecular weight of approximately 13 kilo Daltons and is known to exist as an essential ribosomal protein in protein synthesis. Progress has been made in understanding the complete amino acid sequence of Ribosomal Protein L7/L12 in several microorganisms including Mycoplasma pneumoniae.

The inventors focused on the fact that even though there are similarities between different microorganisms in terms of said molecule, this molecule also has a structural segment that is unique to each microorganism and discovered that, it is possible to detect various microorganisms with species specificity and to detect all serotypes within the same species by using antibody to said protein.

The inventors completed the present invention upon discovering that antibody specific to the protein of Mycoplasma pneumoniae can be obtained and species-specific detection of Mycoplasma pneumoniae is possible using said antibody.

In accordance with the present invention a monoclonal antibody specific to Ribosomal Protein L7/L12 of Mycoplasma pneumoniae has been discovered and developed. The antibody is novel and different than those previously described and has property of reacting specifically to the said protein.

SEQ ID NO:27 and SEQ ID NO:28 in the Sequence List are the DNA sequence of the Ribosomal Protein L7/L12 gene of Mycoplasma pneumoniae and the corresponding amino acid sequence (NCBI database accession # NC_{—}000912). The left terminal and right terminal of the amino acid sequences entered in the Sequence List are amino group (referred to below as the N terminal) and carboxyl group terminals (referred to below as the C terminal), respectively, and the left terminal and right terminal of the base sequence is the 5′ terminal and the 3′ terminal, respectively. Amino acid in the sequence of closest match test is expressed by one letter notation. The notation “+” in closest match test indicates that it is different amino acid but amino acid with similar properties such as hydrophobic. The notation (blank) indicates that it is entirely different amino acid including properties thereof. Moreover, the series of bio-molecular experiments of gene preparation mentioned in this text can be performed by methods entered in standard experimental manuals. “Molecular cloning: A laboratory manual”, Cold Spring Harbor Laboratory Press, Sambrook, J. et al. (1989), is given as an example of the well-known standard experimental manual.

TABLE 10
Closest Match Test:

| | | | |
|---|---|---|---|
| Mp: | 1 | MAKLDKNQLIESLKEMTIMEIDEIIKAVEEAFGVSATPVVAAGAVGGTQEAASEVTVKVT | 60 |
| | 1 | M KLDK QLIESLKEMTI+EIDEIIKAVEEAFGV+ATP+VAAGA G TQEAASEV+VKVT | |
| Mg: | 1 | MGKLDKKQLIESLKEMTIVEIDEIIKAVEEAFGVTATPIVAAGAAGATQEAASEVSVKVT | 60 |
| Mp: | 61 | GYTDNAKLAVLKLYREIAGVGLMEAKTAVEKLPCVVKQDIKPEEAEELKKRFVEVGATVE | 120 |
| | | GY DNAKLAVLKLYREI GVGLMEAKTAVEKLPCVVKQDIKPEEAEELKKRFVEVGATVE | |
| Mg: | 61 | GYADNAKLAVLKLYREITGVGLMEAKTAVEKLPCVVKQDIKPEEAEELKKRFVEVGATVE | 120 |
| Mp: | 121 | IK 122 | |
| | | +K | |
| Mg: | 121 | VK 122 | |

In the present invention, the term “microorganism”, means Mycoplasma pneumoniae, specifically, indicates microorganism having a pathogenic property in respiratory organ and high significance in diagnosis as a causative pathogen of Mycoplasma infections. In the present invention, the term “antibody specifically reacting with microorganism” indicates an antibody that can specifically react with species or genus of microorganism, and antibody specifically reacting with species is especially useful in diagnosis of bacterial infections.

The term “antibody” in the present invention means a polyclonal antibody or monoclonal antibody that can be made using the entire length or only a partial peptide of said Ribosomal Protein L7/L12. Although there are no special restrictions to the peptide length for making the antibody, in the case the antibody corresponds to Ribosomal Protein L7/L12, the segment may be of the length characterizing the Ribosomal Protein L7/L12, and a peptide of 5 amino acids or longer, particularly 8 amino acids or longer, is preferred.

Antiserum containing antibody—(polyclonal antibody) that identifies Ribosomal Protein L7/L12 can be obtained by inoculating laboratory animals with adjuvant and a peptide or the full length protein, as is or, when necessary, after being cross-linked with a carrier protein such as KLH (keyhole-limpet hemocyanin) and BSA (bovine serum albumin) and recovering the serum. Moreover, the antibody can be used after it has been purified from the antiserum. The laboratory animals that are inoculated include sheep, horses, goats, rabbits, mice, rats, etc., and sheep, rabbits, etc., are particularly preferred for preparation of monoclonal antibody. Moreover, monoclonal antibody can also be obtained by conventional methods of making hybridoma cells, but mice are preferred in this case.

The entire length of said protein, or its amino acid sequence of 5 or more, preferably 8 or more, residues that has been fused with glutathione S-transferase (GST), etc., can be purified and used as antigen, or it can be used as antigen without being purified. The antibody can also be produced from the genetic recombination antibody expressed in cultured cell using immunoglobulin genes that have been separated by a variety of methods in published documents (“Antibodies: A Laboratory manual,” E. Harlow et al., Cold Spring Harbor Laboratory), cloning methods, etc.

Antibody to Ribosomal Protein L7/L12 that can be employed as the marker antigen of the present invention can be obtained by the following methods, and other similar methods as well, though not to be limited within these methods:

a) The desired antibody can be acquired by synthesizing a peptide fragment, in the case microorganism has a known Ribosomal Protein L7/L12 genetic sequence and amino acid sequence, using the region least similar to the amino acid sequence of said protein of another microorganism and making polyclonal antibody, or monoclonal antibody, using this peptide fragment as the immune source.

Moreover, it is possible to acquire the entire sequence of said gene by using a conventional genetic procedure, such as gene amplification by PCR using the DNA sequence at both terminals of said known genetic sequence as the probe, or hybridization using the sequence of a homologous segment as the template probe.

Then, a fused gene with another protein gene is constructed and said fused gene is inserted into the host by conventional gene insertion methods using Escherichia coli, etc., as the host and expressed in large quantities. The desired protein antigen can then be acquired by purifying the expressed protein by affinity column methods with antibody to the protein that was used as the fusion protein. In such a case, even if antibody to the amino acid segment retained within the microorganisms is acquired, it does not coincide with the purpose of the present invention because the full length of Ribosomal Protein L7/L12 becomes the antigen. Consequently, hybridoma that produces monoclonal antibody to the antigen that has been obtained by this method is acquired by conventional methods and the desired antibody can be obtained by selecting a clone, which produces antibody that will react only with the desired microorganism.

b) For microorganisms that the amino acid sequence of the Ribosomal Protein L7/L12 is unknown, as the amino acid sequence of the Ribosomal Protein L7/L12 has 50-60% of homology between microorganisms, the protein gene can easily obtained by using a conventional genetic procedure, such as gene amplification of specific sequence moiety by PCR based on the sequence of a homologous segment of the amino acid sequence, or hybridization using the sequence of a homologous segment as the template probe. Then, a fused gene with another protein gene is constructed and said fused gene is inserted into the host such as Escherichia coli, and the like by conventional gene insertion methods, and expressed in large quantities. The desired protein antigen can then be acquired by purifying the expressed protein by affinity column methods with antibody to the protein that was used as the fusion protein. In such a case, even if antibody to the amino acid segment retained within the microorganisms is acquired, it does not coincide with the purpose of the present invention because the full length of Ribosomal Protein L7/L12 becomes the antigen. Consequently, hybridoma that produces monoclonal antibody to the antigen that has been obtained by this method is acquired by conventional methods and the desired antibody can be obtained by selecting a clone, which produces antibody that will react only with the desired microorganism.

c) Alternatively, as another method that is suitable for the case where the amino acid sequence of the Ribosomal Protein L7/L12 is unknown, a peptide of 5 to 30 amino acids corresponding to the common sequence segment retained in the microorganisms is synthesized from the known amino acid sequence of the Ribosomal Protein L7/L12, and polyclonal antibody or monoclonal antibody to this peptide sequence is made by conventional methods. Then Ribosomal Protein L7/L12 highly purified can be obtained by purifying the disrupted liquid of bacterial cells through affinity column chromatography using said antibody. If purity of the protein is insufficient, it can be purified by conventional methods, such as ion exchange chromatography, hydrophobic chromatography, gel filtration, etc., after which the eluted fraction of Ribosomal Protein L7/L12 is identified by method such as western blotting using antibody that was made, and purified protein can be obtained. The desired antibody can be obtained by acquiring hybridoma by conventional methods using the purified Ribosomal Protein L7/L12 antigen that has been obtained, and selecting hybridoma reactable specifically with the desired microorganism.

The antibody of the present invention specific to various microorganisms that has been obtained by the methods in a), b) and c) can be used in a variety of immunoassay methods to provide various diagnostic reagents and kits specific to the desired microorganism. For example, this antibody can be used in aggregation reactions, that is one of known measuring method and where antibody is adsorbed on polystyrene latex particles, ELISA, which is a conventional technology performed in a microtiter plate, conventional immunochromatography methods, and sandwich assay, whereby said antibody labeled with colored particles or particles having coloring capability, or with enzyme or fluorescence substances, and magnetic micro-particles coated with capture antibody, etc., are used, etc.

The term “microorganism diagnosis methods using antibody” means diagnostics methods using any known conventional immunoassay, such as aggregation whereby said antibody is adsorbed on polystyrene latex particles, ELISA, which is a conventional method performed in a microtiter plate, conventional immunochromatography methods, or sandwich assay, whereby said antibody labeled with colored particles or particles having coloring capability, or with enzyme or fluorescence substances, and magnetic micro-particles coated with capture antibody, etc., are used.

Moreover, the optical immunoassay (OIA) technology described in Japanese (via International) Patent Application Laid-open No. 07-509565, in which microorganisms are detected by the principle of an optical interference induced by an antibody reaction on the optical thin film which is formed by silicone, silicon nitride or the like, is a useful as a high sensible diagnostic method, especially as a diagnostic method of microorganisms using an antibody.

Moreover, as a method for extracting intracellular marker antigen from the desired microorganism in aforementioned diagnostic method, reagent treatment using an extraction reagent(s) comprising various surfactants, such as typically Triton X-100 and Tween-20, enzyme treatment using an appropriate enzyme, such as protease, etc., and physical treatment using known cell structure crushing methods, typically cell-crushing of microorganism, can be used. It is preferred that the most suitable conditions for extracting with reagent are set to each kind of microorganism using a proper combination of surfactants, etc.

Moreover, in the present invention the term “reagent kit for diagnosis of microorganisms using antibody” means a diagnostic kit that uses the above-mentioned diagnostic method.

The amino acid and DNA sequence of Ribosomal Protein L7/L12 of Mycoplasma pneumoniae are shown in Sequence List. Consequently, in the case of this microorganism, it is possible to compare the amino acid sequence of Ribosomal Protein L7/L12 with the same protein of closely related microorganisms. Which is shown in Sequence List under the heading “Closest Match”. Synthesizing a peptide with the segment of low homology and making polyclonal or monoclonal antibody to that could short cut the selection of those having specificity to the microorganism.

Especially in the case of a polyclonal antibody, it is preferred that IgG fraction be obtained by purification of the antiserum of immunized laboratory animals with a protein A column, etc., and affinity purification be performed with the synthetic peptide used in immunization of the laboratory animals.

Moreover, PCR primers are formed based on the sequences of N-terminal and C-terminal from the DNA sequence of Ribosomal Protein L7/L12 of the microorganism.

Utilizing homology of the PCR primers, DNA fragments are amplified by the PCR method using genomic DNA and extracted, the fragments of Mycoplasma pneumoniae can be thus acquired according to a conventional method. The entire length of the gene for Ribosomal Protein L7/L12 of Mycoplasma pneumoniae can be acquired through the analysis of the DNA sequence information of these fragments.

The Ribosomal Protein L7/L12 gene of Mycoplasma pneumoniae thus acquired forms a fusion protein gene with, for example, GST, etc., and an expression vector is built using an appropriate expression plasmid, Escherichia coli is transformed and a large quantity of said protein can be expressed. A suitable amount of the transformed Escherichia coli is cultivated and disrupted bacterial fluid is subjected to purification by an affinity column using GST to obtain the GST fusion Ribosomal Protein L7/L12 of Mycoplasma pneumoniae.

It is also possible to acquire the target specific monoclonal antibody by establishing a multiple clone of hybridomas using said protein as is or GST moiety deleted protein as an antigen protein, and selecting the antibody which exhibits a specific response to Mycoplasma pneumoniae bacteria, a homogenized fluid of the bacteria, or Ribosomal Protein L7/L12 of Mycoplasma pneumoniae.

Antibody made based on the present invention can be used in all known types of immunoassay, such as known aggregation reaction whereby said antibody is adsorbed on polystyrene latex particles, ELISA, which is a conventional technology performed in a microtiter plate, conventional immunochromatography, and sandwich assay, whereby said antibody labeled with colored particles or particles that have coloring capability, or enzymes or fluorescence substances, and magnetic particles coated with capture antibody are used, etc.

Moreover, antibody that is made based on the present invention can simultaneously function as a so-called capture antibody that captures said antigen protein in solid or liquid phase and as a detecting antibody that is a so-called enzyme-labeled antibody by modificating an enzyme, such as peroxidase and alkali phosphatase, etc., by conventional methods in any of these immunoassay procedure.

The following examples are given to explain specifically the present invention; the present invention the principle of is not being restricted to these examples.

### Example 15

Cloning of Ribosomal Protein L7/L12 genes from Mycoplasma pneumoniae After inoculating an appropriate amount of Mycoplasma pneumoniae (ATCC15531, distributed and purchased from ATCC) on PPLO agar (DIFCO: 0412-17-3) supplemented with Mycoplasma Supplement (DIFCO: 0836-68-9), the microorganism was cultivated for 5 hours in a CO₂ incubator under conditions of 37° C. and 5% CO₂. The grown colonies were suspended in a TE buffer (Wako Pure Chemical Industries, Ltd.) to a final concentration of approximately 5×10⁹ CFU/ml. Approximately 1.5 ml of this suspension was transferred to a microcentrifuge tube and centrifuged for 2 minutes at 10,000 rpm. The supernatant was discarded. The sediment was resuspended in 567 μl of TE buffer. Then 30 μl of 10% of SDS and 3 μl of 20 mg/ml proteinase K solution were added and thoroughly mixed, followed by incubating for 1 hour at 37° C. The suspension was incubated for further 1 hour at 56° C. After 80 μl of 10% acetyl trimethyl ammonium bromide/0.7 M NaCl solution were added and mixed, it was incubated for 10 minutes at 65° C. Equal volume of chloroform-isoamyl alcohol mixed solution at a volume ratio of 24:1 was added and stirred well.

The solution was centrifuged for 5 minutes at 4° C. and 12,000 rpm with a microcentrifuge and the aqueous fraction was transferred to a new microcentrifuge tube. Isopropanol was added to the fraction at 0.6-times its volume and the tube was vigorously shaken to form sediment of the DNA. The white DNA sediment was scooped with a glass rod and transferred to a different microcentrifugation tube containing 1 ml of 70% ethanol (cooled to −20°). The tube was centrifuged for 5 minutes at 10,000 rpm and the supernatant was gently removed. Then another 1 ml of 70% ethanol was added and the mixture was centrifuged for 5 more minutes.

Once the supernatant had been removed, the sediment was dissolved in 100 μl of TE buffer to obtain the DNA solution. The concentration of the genomic DNA solution was determined quantitatively in accordance with E5: Spectrophotometric determination of the Amount of DNA or RNA, “Molecular cloning: A laboratory manual”, Cold Spring Harbor Laboratory Press, Sambrook, J. et al. (1989). PCR (polymerase chain reaction) was performed using 10 ng of this genomic DNA. Taq polymerase (Takara Co., Ltd., code R001A) was used for PCR. Five ul of buffer attached to the enzyme, 4 μl of a dNTP mixture attached to the enzyme, and 200 pmol of each oligonucleotide (shown in SEQ ID NO:25 and 26 of the Sequence List) were added to the enzyme. Purified water was added to bring the final volume to 50 ul.

The mixture was performed for 5 cycles using TaKaRa PCR Thermal Cycler 480, where one cycle was consisted of treatments for 1 minute at 95° C., 2 minutes at 50° C., and 3 minutes at 72° C. Then, 25 cycles of treatments for 1 minute at 95° C., 2 minutes at 60° C., and 3 minutes at 72° C. per one cycle were carried out. Electrophoresis was performed in 1.5% agarose gel using a part of the PCR product. This product was then stained with ethidium bromide (Nippon Gene Co., Ltd.) and observed under ultraviolet rays to confirm amplification of approximately 400 bp cDNA. After fragmentation treatment with restriction endonucleases BamHI and XhoI, electrophoresis was performed in 1.5% agarose gel and staining with ethidium bromide was carried out. An approximately 400 bp band was cut out from the gel. This band was purified with Suprecol (Takara Co., Ltd.) and then inserted into pGEX-6P-1 (Pharmacia), which is a common vector. The vector can function as an expression vector for the desired molecule, which can express fused protein with GST protein, by insertion of the desired gene fragment into the appropriate restriction endonuclease site.

Actually, vector pGEX-6P-l and the previous DNA were mixed together at a molar ratio of 1:3 and DNA was inserted into the vector with DNA ligase (Invitrogen Co.). Vector pGEX-6P-l into which DNA had been inserted was genetically introduced to Escherichia coli one-shot competent cells and then inoculated in a plate of LBL-broth agar (Takara Co., ltd.) that was a semi-sold culture plate containing 50 μg/ml ampicillin (Sigma). The plate was then incubated at 37° C. for 12 hours and the grown colonies were selected at random and inoculated into L-Broth liquid culture medium containing the same concentration of ampicillin. Shake cultivation was performed at 37° C. for 8 hours and the bacteria were recovered and the plasmid was separated using Wizard Miniprep in accordance with the attached literature. The plasmid was cleaved with restriction endonuclease BamHI/XhoI. Insertion of said PCR product was confirmed by cutting out approximately 370 bp DNA. The DNA base sequence of the DNA that had been inserted was determined using said clone.

Determination of the base sequence of the inserted DNA fragment was performed using the Fluorescence Sequencer of Applied Biosystems. The sequence sample was prepared using PRISM, Ready Reaction Dye Terminator Cycle Sequencing Kit (Applied Biosystems). First, 9.5 ul of reaction stock solution, 4.0 ul of 0.8 pmol/ul T7 promoter primer (Gibco BRL) and 6.5 ul of 0.16 ug/ul template DNA were added to a microtube of 0.5 ml and mixed. After covering the mixture with a double layer of 100 ul of mineral oil, PCR amplification was performed for 25 cycles, where one cycle was consisted of treatments for 30 seconds at 96° C., 15 seconds at 55° C., and 4 minutes at 60° C. The resulting product was then kept at 4° C. for 5 minutes. After the reaction was completed, 80 μl of sterilized pure water was added and stirred. The product was centrifuged and the aqueous layer was extracted 3 times with phenol-chloroform mixed solution. Ten μl of 3M-sodium acetate with pH 5.2 and 300 μl of ethanol were added to 100 μl aqueous layer and stirred. The product was then centrifuged for 15 minutes at room temperature and 14,000 rpm and the sediment was recovered. Once the sediment was washed with 75% ethanol, it was dried under a vacuum for 2 minutes to obtain the sequencing sample. The sequencing sample was dissolved in formamide containing 4 μl of 10 mM EDTA and denatured for 2 minutes at 90° C. This was then cooled in ice and submitted to sequencing.

Two out of the 5 randomly selected clones had homology of the sequence with the probe used for PCR. In addition, a DNA sequence was evidently identical to the gene sequence of Ribosomal Protein L7/L12. The entire base sequence and the corresponding amino acid sequence of the structural gene moiety are as shown in SEQ ID NO:27 and SEQ ID NO:28 of the Sequence List. This gene fragment clearly codes for the gene of Mycoplasma pneumoniae Ribosomal Protein L7/L12.

### Example 16

Mass Expression in Escherichia coil and Purification of Ribosomal Protein L7/L12 from Mycoplasma pneumoniae

Escherichia coli into which expression vector had been inserted was cultivated overnight in 50 ml of LB medium at 37° C. Then 500 ml of 2-times concentrated YT medium was heated at 37° C. for 1 hour. Fifty milliliters of the Escherichia coli solution that had been cultivated overnight were introduced to 500 ml of the aforementioned medium. One hour later, 550 μl of 100 mM isopropyl-β-D(−)-thiogalactopyranoside (IPTG) were introduced and cultivated for 4 hours. The product was then recovered and introduced to 250 ml centrifugation tubes and centrifuged for 10 minutes at 7,000 rpm. The supernatant was discarded and dissolved in 25 ml each of Lysis buffer containing 25% sucrose in 50 mM Tris buffer, pH 7.4. The product was then added with 1.25 ml of 10% NP-40 and 125 μl of 1M MgCl₂, and transferred to a plastic tube. The product was submitted to 5 times of ultrasonic treatment for 1 minute. After that, the product was centrifuged for 15 minutes at 12.000 rpm, and the supernatant was recovered.

Next, the aforementioned supernatant was adsorbed on a glutathione agarose column conditioned with PBS. Then the column was washed using 2 bed volume of a washing solution containing 4.2 mM MgCl₂ and 1 mM dithiothreitol (DTT) in 20 mM Tris buffer, pH 7.4. Elution was performed with 50 mM Tris buffer, pH 9.6, containing 5 mM glutathione. The protein content in the eluted fraction was determined by the pigment bonding method (Bradford method; BioRad Co.) and the main fraction was acquired.

Purity of the purified GST fusion Ribosomal Protein L7/L12 that was obtained was confirmed by electrophoresis to be approximately 75%, showing that purity satisfactory for an immunogen.

### Example 17

Preparation of Monoclonal Antibody to Ribosomal Protein L7/L12 of Mycoplasma pneumoniae First, regarding to immunization of mice, 100 μg of the GST fusion Ribosomal Protein L7/L12 antigen of Mycoplasma pneumoniae were dissolved in 200 μl of PBS and then 200 μl of Freund's complete adjuvant were added and mixed and emulsification was performed. Two hundred microliters of the emulsion were injected intraperitoneally to immunize mice. Then the same emulsion antigen was intraperitoneally injected after 2 weeks, after 4 weeks, and after 6 weeks. Two-fold the concentration of antigen emulsion was injected intraperitoneally after 10 weeks and after 14 weeks. The spleen was excised on 3 days after the final immunization and submitted to cell fusion.

After thoroughly mixing 2×10⁷ myeloma cells per 10⁸ spleen cells from mice, which had been taken out aseptically, in a glass tube, the mixture was centrifuged for 5 minutes at 1,500 rpm and the supernatant was discarded. The cells were thoroughly mixed.

The myeloma cells used for cell fusion were obtained by cultivation of cell strain NS-1 in an RPMI 1640 culture medium containing 10% bovine fetal serum, cultivating this product beginning 2 weeks before cell fusion using an RPMI 1640 medium containing 0.13 mM azaguanine, 0.5 μg/ml MC-210, and 10% bovine fetal serum for 1 weeks, and then further cultivating the cell strain for 1 week in an RPMI 1640 medium containing 10% bovine fetal serum.

Fifty ml of RPMI 1640 culture medium that had been kept at 37° C. were added to the mixed cell sample and centrifuged at 1,500 rpm. After removing the supernatant, 1 ml of 50% polyethylene glycol that had been kept at 37° C. was added and stirred for 1 minute. Ten ml of RPMI 1640 medium kept at 370 were added and the solution was vigorously mixed for approximately 5 minutes while it was suctioned and evacuated from a sterile pipette.

After centrifugation for 5 minutes at 1,000 rpm and removal of the supernatant, 30 ml of HAT culture medium were added to bring the cell concentration to 5×10⁶ cells/ml. This mixture was stirred till uniform and then poured, 0.1 ml per each well, into a 96-well culture plate and cultivated at 37° C. in and under condition of 7% carbon dioxide gas. HAT culture was added, 0.1 ml at a time, on the first day and at after first week and after second week. Then ELISA screened the cells that had produced the desired antibody. GST fusion Ribosomal Protein L7/L12 and GST of were dissolved in PBS containing 0.05% sodium azide and diluted to 10 μg/ml, and the diluted solution was poured, 100 μl each, into separate 96-well plates and adsorbed overnight at 4° C.

After removing the supernatant, 200 μl 1% of bovine serum albumin solution in PBS were added and the mixture was reacted and blocked for 1 hour at room temperature. After removing the supernatant, the product was washed with washing solution containing 0.02% Tween 20, in PBS. One hundred milliliters of culture solution of fused cells were added to this and the mixture was reacted for 2 hours at room temperature. The supernatant was removed and sediment was washed with washing solution. Next, 100 μl of 50 ng/ml peroxidase-labeled goat anti-mouse IgG antibody solution were added and the solution was reacted for 1 hour at room temperature. The supernatant was removed and the product was again washed with washing solution. Then TMB solution (KPL Inc.) was added, 100 μl at a time, and the mixture was reacted for 20 minutes at room temperature. After coloration, 100 μl of 1 N sulfuric acid were added to stop the reaction and absorbance at 450 nm was determined.

As a result, positive wells that only reacted with GST Ribosomal Protein L7/L12, but did not react with GST were detected, and it was concluded that antibody to Ribosomal Protein L7/L12 is produced. Therefore, the cells in the positive wells were recovered and cultivated with HAT medium in a 24-well plastic plate.

The fused medium that had been cultivated was diluted with HT medium to a cell count of approximately 20 cells/ml and then 50 μl of the diluted product was mixed with 10⁶ thymus cells of six-week-old mouse suspended in HT culture medium, in a 96-well culture plate. The mixture was then cultivated for 2 weeks at 37° C. under condition of 7% carbon dioxide gas.

Antibody activity in the culture supernatant was similarly determined by the aforementioned ELISA method and the cells that showed positive reaction with Ribosomal Protein L7/L12 were recovered. Furthermore, the same dilution and cloning procedure was repeated to obtain a total of 5 clones of hybridoma MPRB-1˜5.

### Example 17

Selection of Monoclonal Antibody That Detect Ribosomal Protein L7/L12 of Mycoplasma pneumoniae.

Monoclonal antibody was produced and recovered in accordance with standard methods using the positive hybridoma cells obtained as previously described.

Specifically, 5×10⁶ cells in PBS that had been subcultured using RPMI 1640 culture medium containing 10% FCS were intraperitoneally injected into Balb/C mice that had been intraperitoneally injected with 0.5 ml of Pristane before 2 weeks previously. After 3 weeks, ascites was recovered and the centrifugation supernatant was obtained.

The solution containing antibody that was obtained was adsorbed in a Protein A column (5 ml bed, Pharmacia) and rinsed with PBS at 3-times of the bed volume. Then elution with citrate buffer, pH 3 was performed. The antibody fraction was recovered and the monoclonal antibody that produced by each hybridoma was obtained. The monoclonal antibody derived from these 5 strains of hybridoma was evaluated using ELISA method.

The sandwich assay method was used to assess the monoclonal antibody. The monoclonal antibody that was prepared was used as antibody for detection by being chemically bound to peroxidase. That is, enzyme labeling was performed using horseradish peroxidase (Sigma Grade VI) in accordance with the method described in “Analytical Biochemistry 132 (1983), 68-73” with the reagent S-acetylthioacetic acid N-hydroxysuccinimide for binding. In the ELISA reaction, a solution of commercial anti-Mycoplasma pneumoniae polyclonal antibody (Biodesign, rabbit) diluted to a concentration of 10 μg/ml was poured, 100 μl each, into a separate 96-well plate and adsorbed overnight at 4° C.

After removing the supernatant, 200 μl of 1% bovine serum albumin solution in PBS were added and the mixture was reacted and blocked for 1 hour at room temperature. The supernatant was removed and the product was washed with washing solution containing 0.02% Tween 20, in PBS. One hundred microliters of antigen solution, which had been obtained by adding Triton X-100 to culture solutions of each species of microorganism in an amount to a concentration of 0.3% and then extracting the solution for 5 minutes at room temperature, were added to this and the mixture was reacted for 2 hours at room temperature. The supernatant was removed and the product was further washed with washing solution. Then 100 μl of 5 μg/ml peroxidase-labeled anti-Ribosomal Protein L7/L12 antibody solution were added and the mixture was reacted for 1 hour at room temperature. The supernatant was removed and the product was washed again with washing solution. TMB solution (KPL Inc.) was added, 100 μl each, and the mixture was reacted for 20 minutes at room temperature. After coloration, 100 μl of 1 N sulfuric acid were added to stop the reaction. Absorbance at 450 nm was determined.

It is clearly evident that when monoclonal antibody derived from hybridoma MPRB-l was used as the enzyme-labeled antibody, all strains of Mycoplasma pneumoniae tested were detected at a sensitivity of 10⁶ cells/ml, while reactivity of other microorganisms e.g., Haemophilus influenzae, Klebsiella pneumoniae, Chlamydia pneumoniae and Neisseria meningitides could not exhibit reactivity, even at high concentrations of 10⁸ cells/ml and therefore, antibody with specific reactivity to Mycoplasma pneumoniae can be obtained by using monoclonal antibody to Ribosomal Protein L7/L12. The antibody was named as AMMP-1. Table 11 shows only those results with AMMP-1. Results with other antibodies, which cross-reacted with other microorganisms are not mentioned here.

TABLE 11

| | Result of Detection |
|---|---|
| | (10⁶ cells/ml) |
| N. pneumoniae | + |
| | Results of Detection |
| | (10⁸ cells/ml) |
| N. meningitides | ˜ |
| N. lactamica | ˜ |
| N. mucosa | ˜ |
| N. sicca | ˜ |
| H. influenzae | ˜ |
| B. catarrharis | ˜ |
| N. gonorrhoeae | ˜ |
| E. coli | ˜ |
| K. pneumoniae | ˜ |

(+: Positive, ˜: Negative)

### Example 17

Acquisition of a polyclonal antibody, which specifically reacts with Ribosomal Protein L7/L12 of Mycoplasma pneumoniae using a Ribosomal Protein L7/L12-immobilized affinity column The Ribosomal Protein L7/L12 of Mycoplasma pneumoniae, which was acquired by the method described in Examples 1, or the supernatant of Mycoplasma pneumoniae bacteria treated with Triton X-100 was used as an antigen. About 1.2 ml of a physiological saline solution containing 100 μg of antigen was emulsified with the addition of 1.5 ml of Freund's adjuvant. The emulsion was subcutaneously injected into SPF Japanese White Rabbit to immunize the animal. The rabbit was immunized 5 to 6 times, once every two weeks, and the antibody titer was confirmed.

The antibody titer was confirmed by the ELISA method. Solutions of Ribosomal Protein L7/L12 of Mycoplasma pneumoniae dissolved in PBS containing 0.05% sodium azide diluted to 10 μg/ml were poured, 100 μl each, into 96-well plates and adsorbed overnight at 4° C. After removing the supernatant, 200 μl of 1% bovine serum albumin solution in PBS were added and the mixture was reacted and blocked for 1 hour at room temperature. The supernatant was removed and the product was washed with a washing solution containing 0.02% Tween 20, in PBS. One hundred μl of a solution obtained by diluting normal rabbit serum and immunized rabbit antiserum was added and the mixture was reacted for two hours at room temperature. The supernatant was removed and the product was further washed with a washing solution. Then, 100 μl of 50 ng/ml peroxidase-labeled goat anti-rabbit IgG antibody solution was added and the mixture was reacted for one hour at room temperature. The supernatant was removed and the product was washed again with a washing solution. OPD solution (Sigma Co.) was added, 100 μl each, and the mixture was reacted for 20 minutes at room temperature. After coloration, 100 μl of 1 N sulfuric acid was added to stop the reaction. Absorbance at 492 nm was determined.

After confirming that the antibody titer had increased, a large quantity of blood was collected. Blood was collected in a glass centrifuge tube from the ear artery, allowed to stand for one hour at 37° C., and then overnight at 4° C. The mixture was centrifuged at 3000 rpm for 5 minutes and the supernatant was recovered. The resulting anti-serum was preserved at 4° C.

An affinity column immobilized Ribosomal Protein L7/L12 of Mycoplasma pneumoniae was prepared. HiTrap NHS-activated column (1 ml, manufactured by Pharmacia) was used. Immediately after replacing the column with 1 mM HCl, a PBS solution of Ribosomal Protein L7/L12 (1 mg/ml) was charged. The column was allowed to stand for 30 minutes and a blocking reagent was charged, followed by equilibration with PBS.

Using the affinity column immobilized Ribosomal Protein L7/L12 of Mycoplasma pneumoniae, the polyclonal antibody in the resulting anti-serum obtained as an antigen from the supernatant of Triton X-100 treated bacteria of Mycoplasma pneumoniae as an antigen was purified. This antiserum was diluted with PBS to a volume of 5 times, passed through a 0.45 μm filter, then adsorbed in the column immobilized with Ribosomal Protein L7/L12 of Mycoplasma pneumoniae at a flow rate of 0.5 ml/min. After elution from the column with 0.1 M glycine buffer, pH 2.1 and immediately neutralizing with 1 M Tris buffer, pH 9.0, eluted fractions of the target antibody were recovered by the ELISA method, the same as the antibody titer measuring method.

The polyclonal antibody obtained in this manner was evaluated by the same OIA method as described in Japanese Patent Application Laid Open No.07-509565.

The purified antibody was used as a capture antibody for the OIA method. Moreover, AMMP-1 monoclonal antibody described in Example 4 was labeled with peroxidase and used as the detect antibody. The enzyme labeling was performed using horseradish peroxidase (Sigma Grade VI) and the reagent S-acetylthioacetic acid N-hydroxysuccinimide for binding in accordance with the method in “Analytical Bio-chemistry 132 (1983), 68-73”. In the OIA reaction, the purified polyclonal antibody in PBS containing 0.05% sodium azide was diluted with 0.1 M HEPES buffer, pH 8.0, to a concentration of 10 μg/ml and the diluted solution was added onto a silicon wafer, 50 μl at a time, to react for 30 minutes at room temperature, followed by washing with distilled water and coated with a coating solution including sucrose and alkali-treated casein. Fifteen μl of antigen solution, which had been obtained by adding Triton X-100 to culture solutions of various species of microorganism to a concentration of 0.5% and then extracting for 5 minutes at room temperature, was added onto the above-described silicon wafer and reacted for 10 minutes at room temperature. Then, 15 μl of 20 μg/ml peroxidase-labeled monoclonal antibody was added and the mixture was reacted for 10 minutes. After washing with distilled water, TMB solution (KPL Inc.) was added, 15 μl at a time, and the mixture was reacted for 5 minutes at room temperature. The product was washed with distilled water and observed the blue colored as a result of enzyme reaction.

As shown in Table 12 as a result, it is clear that when the purified polyclonal antibody of APMP-1 is used as the capture antibody, Mycoplasma pneumoniae can be detected at a sensitivity of 10⁸ cells/ml, while reactivity of other microorganisms cannot be detected. Thus, it was confirmed that an affinity column immobilized with the Ribosomal Protein L7/L12 of Mycoplasma pneumoniae obtained the polyclonal antibody with specific reactivity to Mycoplasma pneumoniae.

TABLE 12

Results of Detection
(10⁸ cells/ml)

| | | |
|---|---|---|
| N. pneumoniae | | + |
| H. influenzae | ATCC10211 | ˜ |
| E. coli | ATCC25922 | ˜ |
| E. faecalis | ATCC19433 | ˜ |
| K. pneumoniae | ATCC13883 | ˜ |
| N. gonorrhoeae | IID821 | ˜ |
| N. lactamica | ATCC23970 | ˜ |
| N. meningitidis | ATCC13090 | ˜ |
| P. aeruginosa | ATCC27853 | ˜ |
| Group B | ATCC12386 | ˜ |
| Streptococcus | | ˜ |
| S. aureus | ATCC25923 | ˜ |
| S. pneumoniae | ATCC27336 | ˜ |
| S. pyogenes | ATCC19615 | ˜ |

(+: Positive, ˜: Negative)

### INDUSTRIAL APPLICABILITY

According to the present invention, not can only microorganisms be detected according to species, but microorganisms of all serotypes in the same species can be detected at a high precision, by using antibodies to intracellular molecules of the same function.

By using antibodies to ribosomal proteins L7/L12 of various microorganisms as such antibodies, Haemophilus influenzae and Neisseria gonorrhoeae can be detected precisely.

Moreover, specific antibodies used for the detection of various kinds of microorganisms can be prepared by using intracellular molecules exhibiting same functions in various microorganisms as an antigen.

By using antibodies to Ribosomal Proteins L7/L12 of microorganisms as such antibodies, Chlamydia pneumoniae can be detected precisely.

Moreover, detection of microorganisms can be performed with higher precision and wider applicability by using the reagent kit for detecting microorganisms comprising such an antibody.

### REFERENCES CITED

### PATENT DOCUMENTS

U.S. Pat. No. 5,008,186, Grayston, et al. 1991 Detection of unique Chlamydia strain associated with acute respiratory disease

U.S. Pat. No. 5,281,518 Campbell, et al. 1994 Detection of a unique Chlamydia strain associated with acute respiratory disease

U.S. Pat. No. 5,350,673 Campbell, et al. 1994 Detection of a unique Chlamydia strain associated with acute respiratory disease

U.S. Pat. No. 5,552,279 Weisburg, et al. Sep. 3, 1996. Nucleic acid probes for the detection of Mycoplasma pneumoniae and Mycoplasma genitalium.

EPO 305145, Application No. 88307793.5 Zivin and Monahan. Mar. 1, 1989. Methods and probes for detecting nucleic acid.

EPO 250662, Application No. 86304919.3. Gobel and Stanbridge. Jan. 7, 1988. Detection of Mycoplasma by DNA hybridization.

Japanese Patent Application Laid Open No. 63(1988)-298

### OTHER DOCUMENTS:

Batteiger, B. E., W. J. t. Newhall, et al. (1986). “Antigenic analysis of the major outer membrane protein of Chlamydia trachomatis with murine monoclonal antibodies.” Infect Immun 53(3): 646-50.

Bernet, C., M. Garret, et al. (1993). “Detection of Mycoplasma pneumoniae by using the polymerase chain reaction.” J Clin Microbiol 31(4): 1013-5.

Buck, G. E., L. C. O'Hara, et al. (1993). “Rapid, sensitive detection of Mycoplasma pneumoniae in simulated clinical specimens by DNA amplification.” J Clin Microbiol 31(4): 1013-5.

Chan E D, Kalayanamit T, Lynch D A, Tuder R, Arndt P, Winn R, Schwarz M I. “Mycoplasma pneumoniae-associated bronchiolitis causing severe restrictive lung disease in adults: report of three cases and literature review”. Chest. 1999;115(4): 1188-94.

Cimolai N. Mycoplasma pneumoniae respiratory infection. Pediatr Rev. 1998; 19(10): 327-31.

Cles, L. D. and W. E. Stamm (1990). “Use of HL cells for improved isolation and passage of Chlamydia pneumoniae.” J Clin Microbiol 28(5): 938-40.

Essig, A., P. Zucs, et al. (1997). “Diagnosis of ornithosis by cell culture and polymerase chain reaction in a patient with chronic pneumonia.” Clin Diagn Lab Immunol 4(2): 213-6.

Gobel, U. B., A. Geiser, et al. (1987). “Oligonucleotide probes complementary to variable regions of ribosomal RNA discriminate between Mycoplasma species”.

Godzik, K. L., E. R. O'Brien, et al. (1995). “In vitro susceptibility of human vascular wall cells to infection with Chlamydia pneumoniae.” J Clin Microbiol 33(9): 2411-4.

Granato, P. A., L. Poe, et al.(1980). “Use of modified New York City medium for growth of Mycoplasma pneumoniae. A preliminary report.” Zh Mikrobiol Epidemiol Immunobiol (8): 50-3.

Harlow, E., and D. Lane (1988). “Antibodies: A laboratory manual.” New York. Cold Spring Harbor Laboratory Press.

Hyman, H. C., D. Yogev, et al.(1987). “DNA probes for detection and identification of Mycoplasma pneumoniae and Mycoplasma genitalium” Hyman, C. L., P. M. Roblin, et al. (1995). “Prevalence of asymptomatic nasopharyngeal carriage of Chlamydia pneumoniae in subjectively healthy adults: assessment by polymerase chain reaction-enzyme immunoassay and culture.” Clin Infect Dis 20(5): 1174-8.

Jensen, J. S., J. Sondergard-Andersen, et al. (1993). “Detection of Mycoplasma pneumoniae in simulated clinical samples by polymerase chain reaction. Brief report.” J Med Microbiol 38(3): 166-70.

Kai, M., S. Kamiya, et al. (1987). “Rapid detection of Mycoplasma pneumoniae in clinical samples by the polymerase chain reaction.” J Gen Microbiol 133(Pt 7): 1969-74.

Knudson, D. L. and R. MacLeod (1979). “Mycoplasma pneumoniae and Mycoplasma salivarium: electron microscopy of colony growth in agar.” Sci Rep Res Inst Tohoku Univ [Med] 26(3-4): 71-91.

Kuo, C. C. and J. T. Grayston (1990). “A sensitive cell line, HL cells, for isolation and propagation of Chlamydia pneumoniae strain TWAR.” J Infect Dis 162(3): 755-8.

Madsen R D, Weiner L B, McMillan J A, Saeed F A, North J A, Coates S R. (1988). Direct detection of Mycoplasma pneumoniae antigen in clinical specimens by a monoclonal antibody immunoblot assay. Am J Clin Pathol 89(1):95-9

Peterson, E. M., X. Cheng, et al. (1993). “Functional and structural mapping of Chlamydia trachomatis species-specific major outer membrane protein epitopes by use of neutralizing monoclonal antibodies.” J Gen Microbiol 139(Pt 11): 2621-6.

Peterson, E. M., L. M. de la Maza, et al. (1998). “Characterization of a neutralizing monoclonal antibody directed at the lipopolysaccharide of Chlamydia pneumoniae.” Scand J Infect Dis 30(4): 381-6.

Ragnar Norrby, S. (1999). “Atypical pneumonia in the Nordic countries: aetiology and clinical results of a trial comparing fleroxacin and doxycycline. Nordic Atypical Pneumonia Study Group.” J Med Microbiol 48(12): 1115-22.

Thom, D. H. and J. T. Grayston (1991). “Infections with Chlamydia pneumoniae strain TWAR.” Clin Chest Med 12(2): 245-56.

Tilton, R. C., F. Dias, et al. (1980). “DNA probe versus culture for detection of Mycoplasma pneumoniae in clinical specimens.” J Clin Microbiol 12(6): 748-52.

Verkooyen, R. P., N. A. Van Lent, et al. (1998). “Diagnosis of Chlamydia pneumoniae infection in patients with chronic obstructive pulmonary disease by micro-immunofluorescence and ELISA.” Am Heart J 135(1): 15-20.

Yogev, D., D. Halachmi, et al. (1988). “Distinction of species and strains of mycoplasmas (mollicutes) by genomic DNA fingerprints with an rRNA gene probe.” J Clin Microbiol 26(11): 2266-9.

Yoshizawa, H., K. Dairiki, et al. (1992). “Comparison of sensitivity of Hep-2 cells with that of HL cells against Chlamydia pneumoniae.” Kansenshogaku Zasshi 66(8): 1037-41.

NCBI database accession #NC_{—}000922. Kalman, S., Mitchell, W., Marathe, R., Lammel, C., Fan, J., Olinger, L., Grimwood, J., Davis, R. W. and Stephens, R. S.

NCBI database accession #AE001593.1. Kalman, S., Mitchell, W., Marathe, R., Lammel, C., Fan, J., Olinger, L., Grimwood, J., Davis, R. W. and Stephens, R. S.

NCBI database accession #NC#000912., Himmelreich,R., Hilbert, H., Plagens, H., Pirkl, E., Li, B. C. and Herrmann, R.

Remarks Concerning Deposited Microorganisms

The organization in which the microorganisms have been deposited:

National Institute of Bioscience and Human Technology, the Agency of Industrial Science and Technology

Address: 1-1-3, Higashi, Yatabe-cho, Tsukuba-shi,

Ibaraki-ken, Japan (Postal Code: 305).

Date of deposition: Jul. 28, 1999

Number of deposition given by the deposition organization: FERM PB6807

What is claimed is:

1. Antibodies which are antibodies to ribosomal protein of microorganisms and which react specifically with said microorganisms.

2. The antibodies according to claim 1, where the ribosomal protein of the microorganisms is ribosomal protein L7/L12.

3. The antibodies according to claim 1, wherein said microorganisms are microorganisms which cause a sexually transmitted disease (STD).

4. The antibodies according to claim 1, where said microorganisms are microorganisms which cause respiratory tract infections.

5. The antibody according to claim 4, wherein said microorganism is Haemophilus influenzae.

6. The antibody according to claim 4, wherein said microorganism is Streptococcus pneumoniae.

7. The antibody according to claim 3, wherein said microorganism is Neisseria gonorrhoeae.

8. The antibody according to claim 3, wherein said antibody is an antibody to ribosomal protein L7/L12 of Neisseria gonorrhoeae and which identifies a continuous amino acid sequence moiety from 5 to 30 amino acids including the 115th alanine in the amino acid sequence of Sequence ID No. 22.

9. A method of detecting microorganisms, comprising contacting the antibody according to claim 1 with said microorganism, wherein said antibody is an antibody to intracellular molecules that have the same function for a variety of microorganisms used.

10. A method of detecting microorganisms, comprising contacting an antibody according to claim 1 with said microorganism.

11. A reagent kit for detecting microorganisms, which is characterized by the fact that an antibody to intracellular molecules that have the same function for a variety of microorganisms is used.

12. A reagent kit for detecting microorganisms, comprising the antibody according to claim 1.

13. A method of preparing an antibody according to claim 1, characterized by the fact that ribosomal protein L7/L12 protein of microorganisms obtained by a gene manipulation procedure or by isolation from microorganisms, peptide moiety thereof, or a synthesized peptide corresponding to the peptide moiety is used as an immune source.

14. The antibodies according to claim 1, wherein said microorganisms is Mycoplasma pneumoniae.

15. The antibodies according to claim 1, wherein said microorganisms is Chlamydia pneumoniae.

16. An antibody which is antibody to ribosomal protein of microorganism belonging to Chlamydia pneumoniae and which reacts specifically with ribosomal protein of said microorganism.

17. The antibody according to claim 16, wherein the ribosomal protein of the microorganism belonging to Chlamydia pneumoniae is Ribosomal Protein L7/L12.

18. The antibody according to claim 16, wherein the antibody is monoclonal antibody or polyclonal antibody.

19. The antibody according to claim 16, wherein the antibody is conjugated with an enzyme.

20. A method for detecting Chlamydia pneumoniae, comprising contacting said Chlamydia pneumoniae with an antibody according to claim 16.

21. A method of detecting the microorganism belonging to Chlamydia pneumoniae, comprising:
a) contacting a test sample with a lysing solution, and extracting ribosomal protein from the microorganism
b) contacting the extracted test sample with a captured antibody, wherein said antibody is an antibody according to claim 16, fixed on a solid surface, and forms an antigen-antibody complex from the ribosomal protein and the capture antibody, and
c) detecting the antigen-antibody complex using a detection antibody, wherein the detection antibody is an antibody according to claim 16.

22. The method according to claim 16, wherein the detection antibody is an antibody conjugated with an enzyme and the antigen-antibody complex is detected with a substrate specific to the enzyme.

23. A reagent kit for detecting the microorganism belonging to Chlamydia pneumoniae, comprising the antibody according to claim 16.

24. A method of preparing the antibody according to claim 16, characterized by using as an immunogen, Ribosomal Protein L7/L12 of microorganism belonging to Chlamydia pneumoniae and obtained by a gene manipulation procedure or by isolation and purification from the microorganism, a peptide moiety thereof or a synthesized peptide corresponding to the peptide moiety.

25. An antibody which is antibody to ribosomal protein of microorganism belonging to Mycoplasma pneumoniae and which reacts specifically with ribosomal protein of said microorganism.

26. The antibody according to claim 25, wherein the ribosomal protein of the microorganism belonging to Mycoplasma pneumoniae is Ribosomal Protein L7/L12.

27. The antibody according to claim 25, wherein the antibody is a monoclonal antibody or polyclonal antibody.

28. The antibody according to claim 25, wherein the antibody is conjugated with an enzyme.

29. A method of detecting the microorganism belonging to Mycoplasma pneumoniae, comprising contacting said Mycoplasma pneumoniae with an antibody according to claim 25.

30. A method of detecting the microorganism belonging to Mycoplasma pneumoniae in a test sample, comprising:
a) contacting a test sample with a lysing solution, and extracting ribosomal protein from the microorganism
b) contacting the extracted test sample with a capture antibody, wherein the capture antibody is an antibody according to claim 25, fixed on a solid surface, and forming an antigen-antibody complex from the ribosomal protein and the capture antibody, and
c) detecting the antigen-antibody complex using a detection antibody, wherein the detection antibody is an antibody according to claim 25.

31. The method according to claim 30, wherein the detection antibody is an antibody conjugated with an enzyme and the antigen-antibody complex is detected with a substrate specific to the enzyme.

32. A reagent kit for detecting the microorganism belonging to Mycoplasma pneumoniae, comprising an antibody according to claim 25.

33. A method of preparing the antibody defined according to claim 25, characterized by using as an immunogen, Ribosomal Protein L7/L12 of microorganism belonging to Mycoplasma pneumoniae and obtained by a gene manipulation procedure or by isolation and purification from the microorganism, a peptide moiety thereof or a synthesized peptide corresponding to the peptide moiety.


SEQUENCE LISTING (30 sequences)

SEQ ID NO: 1 - DNA, 369 bp, Haemophilus influenzae
  CDS (1)..(369)
atgtcattaactaacgaacaaatcattgaagcgattgcttcaaaaactgtaactgaaatcgttgaattaatcgcagcgatggaagaaaaattcggtgtttcagcagcggcagcagtagcagcagctccagcagcaggcggtgcagcggcagcagaagaaaaaactgaattcgacgttgtacttaaatctgcaggtgcgaacaaagtagcagtaattaaagcagtacgtggtgcaactggtttaggcttaaaagaagctaaagatttagttgaatctgctccagctaacttaaaagaaggcgtttctaaagaagaagctgaagcacttaagaaagaattagaagaagcgggtgcagaagtagaagttaaa

SEQ ID NO: 2 - protein, 123 aa, Haemophilus influenzae
MSLTNEQIIEAIASKTVTEIVELIAAMEEKFGVSAAAAVAAAPAAGGAAAAEEKTEFDVVLKSAGANKVAVIKAVRGATGLGLKEAKDLVESAPANLKEGVSKEEAEALKKELEEAGAEVEVK

SEQ ID NO: 3 - DNA, 375 bp, Helicobacter pylori
  CDS (1)..(375)
atggcaatttcaaaagaagaagtgttagagtatattggttcattgagcgttttagagctttctgaattggttaaaatgtttgaggaaaaatttggcgtgagcgcgactccaacggtcgtagcgggtgcggctgtagctggcggtgcagcggctgagagcgaagaaaaaaccgaatttaatgtgattttggccgatagcggtgctgaaaaaattaaggtgattaaagtggttcgtgaaatcactggacttggcctgaaagaagctaaagacgctaccgaaaaaacccctcatgtgcttaaagagggcgtgaataaagaagaagctgaaaccatcaagaagaaacttgaagaagtaggcgctaaggttgaagtcaag

SEQ ID NO: 4 - protein, 125 aa, Helicobacter pylori
MAISKEEVLEYIGSLSVLELSELVKMFEEKFGVSATPTVVAGAAVAGGAAAESEEKTEFNVILADSGAEKIKVIKVVREITGLGLKEAKDATEKTPHVLKEGVNKEEAETIKKKLEEVGAKVEVK

SEQ ID NO: 5 - DNA, 366 bp, Streptococcus pneumoniae
  CDS (1)..(366)
atggcattgaacattgaaaacattattgctgaaattaaagaagcttcaatccttgaattgaacgaccttgtaaaagctatcgaagaagaatttggtgtaactgcagctgctcctgtagctgttgctgcagctgatgcagctgatgctggtgctgctaaagattcattcgacgttgaattgacatctgcaggcgacaaaaaagttggcgttatcaaagttgtacgtgaaatcactggtcttggtcttaaagaagctaaagaacttgttgacggtgcaccagcacttgttaaagaaggcgttgcaactgcagaagctgaagaaatcaaagctaaattggaagaagctggagcttcagttactcttaaa

SEQ ID NO: 6 - protein, 122 aa, Streptococcus pneumoniae
MALNIENIIAEIKEASILELNDLVKAIEEEFGVTAAAPVAVAAADAADAGAAKDSFDVELTSAGDKKVGVIKVVREITGLGLKEAKELVDGAPALVKEGVATAEAEEIKAKLEEAGASVTLK

SEQ ID NO: 7 - DNA, 369 bp, Neisseria gonorrhoeae
  CDS (1)..(369)
atggctattactaaagaagacattttggaagcagttggttctttgaccgtaatggaattgaatgacctggttaaagcttttgaagaaaaattcggtgtttctgctgctgctgttgcagttgcaggtcctgctggtgccggtgctgccgatgctgaagaaaaaaccgaatttgatgtcgttttggcttctgccggcgatcaaaaagtcggcgtgattaaagttgtccgtgcaattactggtttgggtctgaaagaagctaaagacatcgttgacggcgcacctaaaaccattaaagagggtgtttctaaagctgaagccgaagacatccaaaaacaactggaagcagcaggcgctaaagtcgaaatcaaa

SEQ ID NO: 8 - protein, 123 aa, Neisseria gonorrhoeae
MAITKEDILEAVGSLTVMELNDLVKAFEEKFGVSAAAVAVAGPAGAGAADAEEKTEFDVVLASAGDQKVGVIKVVRAITGLGLKEAKDIVDGAPKTIKEGVSKAEAEDIQKQLEAAGAKVEIK

SEQ ID NO: 9 - DNA, 369 bp, Neisseria meningitidis
  CDS (1)..(369)
atggctattactaaagaagacattttggaagcagttggttctttgaccgtaatggaattgaacgacttggttaaagcttttgaagaaaaattcggtgtttctgctgctgctgttgcagttgcaggtcctgctggtgccggtgctgccgatgctgaagaaaaaaccgaatttgatgtcgttttggcttctgccggtgatcaaaaagtcggcgtgattaaagttgtccgtgcaattaccggtttgggtctgaaagaagctaaagacatcgttgacggtgcacctaaaaccattaaagagggtgtttctaaagctgaagccgaagacatccaaaaacaactggaagaagccggcgctaaagtcgaaatcaaa

SEQ ID NO: 10 - protein, 123 aa, Neisseria meningitidis
MAITKEDILEAVGSLTVMELNDLVKAFEEKFGVSAAAVAVAGPAGAGAADAEEKTEFDVVLASAGDQKVGVIKVVRAITGLGLKEAKDIVDGAPKTIKEGVSKAEAEDIQKQLEEAGAKVEIK

SEQ ID NO: 11 - DNA, 31 bp, Artificial Sequence
  Description of Artificial Sequence Primer
gtaaggatccatgtcattaactaacgaacaa

SEQ ID NO: 12 - DNA, 34 bp, Artificial Sequence
  Description of Artificial Sequence Primer
agcatctcgagatttaacttctacttctgcaccc

SEQ ID NO: 13 - DNA, 33 bp, Artificial Sequence
  Description of Artificial Sequence Primer
ggaaggatccatggcattgaacattgaaaacat

SEQ ID NO: 14 - DNA, 33 bp, Artificial Sequence
  Description of Artificial Sequence Primer
tactctcgagtttaagagtaactgaagctccag

SEQ ID NO: 15 - DNA, 31 bp, Artificial Sequence
  Description of Artificial Sequence Primer
gtaaggatccatggctattactaaagaagac

SEQ ID NO: 16 - DNA, 34 bp, Artificial Sequence
  Description of Artificial Sequence Primer
agcatctcgagatttgatttcgactttagcgcct

SEQ ID NO: 17 - DNA, 369 bp, Haemophilus influenzae; identical to SEQ ID NO: 1
  CDS (1)..(369)

SEQ ID NO: 18 - protein, 123 aa, Haemophilus influenzae; identical to SEQ ID NO: 2

SEQ ID NO: 19 - DNA, 366 bp, Streptococcus pneumoniae; identical to SEQ ID NO: 5
  CDS (1)..(366)

SEQ ID NO: 20 - protein, 122 aa, Streptococcus pneumoniae; identical to SEQ ID NO: 6

SEQ ID NO: 21 - DNA, 369 bp, Neisseria gonorrhoeae; identical to SEQ ID NO: 7
  CDS (1)..(369)

SEQ ID NO: 22 - protein, 123 aa, Neisseria gonorrhoeae; identical to SEQ ID NO: 8

SEQ ID NO: 23 - DNA, 390 bp, Chlamydia pneumoniae
  CDS (1)..(390)
gtgacaacagaaagtttggaaactttagtagagaagttaagtaatttaactgtactagaactctctcaattgaaaaaattattagaagagaagtgggatgttactgcttctgctcccgtagttgctgttgctgctggtggtggcggagaagctcctgttgctgccgaacctacagaatttgcagtaaccctcgaagatgttcctgcagataaaaaaatcggcgtcttaaaagtcgttagggaagtaactggattagctttaaaagaagctaaagaaatgacagaaggtttacctaaaactgttaaagaaaaaacttctaaaagtgatgctgaagatactgttaagaagttacaagatgctggcgcaaaagcctcatttaagggactgtaa

SEQ ID NO: 24 - protein, 129 aa, Chlamydia pneumoniae
VTTESLETLVEKLSNLTVLELSQLKKLLEEKWDVTASAPVVAVAAGGGGEAPVAAEPTEFAVTLEDVPADKKIGVLKVVREVTGLALKEAKEMTEGLPKTVKEKTSKSDAEDTVKKLQDAGAKASFKGL

SEQ ID NO: 25 - DNA, 33 bp, Artificial Sequence
  Description of Artificial Sequence Primer
gtgggatccgtgacaacagaaagtttggaaact

SEQ ID NO: 26 - DNA, 35 bp, Artificial Sequence
  Description of Artificial Sequence Primer
ttactcgagttacagtcccttaaatgaggcttttg

SEQ ID NO: 27 - DNA, 369 bp, Mycoplasma pneumoniae
  CDS (1)..(369)
atggcaaaactagataaaaaccaattaattgaatcgttgaaggaaatgaccatcatggaaatcgatgaaatcattaaggctgtagaagaagcttttggagtatcggcaacacctgtagtagctgctggtgctgttggtggtacacaagaagctgctagcgaagtgactgtgaaagttactggttacactgacaacgctaaattagctgtgttaaagctttaccgcgaaattgctggtgttggtttaatggaagctaaaactgctgtggaaaaacttccttgtgttgttaagcaagacatcaaacctgaagaagctgaagaacttaaaaagcgtttcgttgaagttggagcaactgttgaaatcaaataa

SEQ ID NO: 28 - protein, 122 aa, Mycoplasma pneumoniae
MAKLDKNQLIESLKEMTIMEIDEIIKAVEEAFGVSATPVVAAGAVGGTQEAASEVTVKVTGYTDNAKLAVLKLYREIAGVGLMEAKTAVEKLPCVVKQDIKPEEAEELKKRFVEVGATVEIK

SEQ ID NO: 29 - DNA, 29 bp, Artificial Sequence
  Description of Artificial Sequence Primer
aatggatccatggcaaaactagataaaaa

SEQ ID NO: 30 - DNA, 30 bp, Artificial Sequence
  Description of Artificial Sequence Primer
tgactcgagttatttgatttcaacagttgc